(19) United States
(12) Patent Application Publication
(10) Pub. No.: US 2019/0362809 A1
(43) Pub. Date: Nov. 28, 2019

(54) JOINT ANALYSIS OF MULTIPLE HIGH-DIMENSIONAL DATA USING SPARSE MATRIX APPROXIMATIONS OF RANK-1

(71) Applicants: UNIVERSITY OF HAWAII, Honolulu, HI (US); SNR ANALYTICS INC., Kaneohe, HI (US)

(72) Inventors: Gordon S. Okimoto, Honolulu, HI (US); Thomas M. Wenska, Kaneohe, HI (US)

(21) Appl. No.: 16/241,899

(22) Filed: Jan. 7, 2019

Related U.S. Application Data
(63) Continuation of application No. PCT/US2017/041230, filed on Jul. 7, 2017.
(60) Provisional application No. 62/490,529, filed on Apr. 26, 2017.
(60) Provisional application No. 62/360,201, filed on Jul. 8, 2016.

(51) Int. Cl. G16B 25/00; G16B 40/00; G16H 50/20; G06N 20/00

(52) U.S. Cl. CPC G16B 25/00; G06N 20/00; G16H 50/20; G16B 40/00

(57) ABSTRACT

Disclosed herein are systems and methods for joint analysis of multiple high-dimensional data types using sparse matrix approximations of rank−1. In some embodiments, a method comprises determining a signal of interest (SOI) that is shared by a plurality of type-specific signatures for a plurality of data types; and determining a sparse linear model of the shared SOI based on non-zero entries of a plurality of sparse eigenarrays.

### RELATED APPLICATIONS

The present application is a continuation of PCT Application No. PCT/US2017/041230, filed on Jul. 7, 2017, which claims priority to U.S. Provisional Application No. 62/360,201, filed on Jul. 8, 2016; and U.S. Provisional Application No. 62/490,529, filed on Apr. 26, 2017. The content of each of these related applications is incorporated herein by reference in its entirety.

### STATEMENT REGARDING FEDERALLY SPONSORED R&D

This invention was made with government support under grant P30 CA071789 and R01 CA161209 awarded by the National Institutes of Health. The government has certain rights in the invention.

### BACKGROUND

### Field

The present disclosure relates generally to the field of diagnosing and treating diseases and more particularly to joint analysis of multiple high-dimensional data using sparse matrix approximation of rank-1 for diagnosing and treating diseases.

### Description of the Related Art

A rapidly expanding backlog of multi-modal data obtained from a common set of bio-samples has shifted the translational bottleneck in disease research (e.g., cancer research) from data acquisition to data analysis and interpretation. The current lack of software and algorithms for the analysis of multi-modal data has impeded the discovery of new approaches for diagnosing and treating cancer and other complex diseases.

### SUMMARY

Disclosed herein are systems and methods for joint analysis of multiple high-dimensional data types using sparse matrix approximations of rank-1. In some embodiments, a method comprises: receiving multi-modal data sets (MMDS), wherein the multi-modal data sets comprise a plurality of data matrices of a plurality of data types; generating a super-matrix comprising the plurality of data matrices; determining a sparse rank-1 approximation of the super-matrix; determining a sparse multi-modal signature (MMSIG) of the super-matrix from the sparse rank-1 approximation of the super-matrix; parsing the sparse multi-modal signature of the super-matrix to determine a plurality of type-specific signatures for the plurality of data types; parsing the sparse rank-1 approximation of the super-matrix to determine a plurality of sparse eigen-arrays for the plurality of data matrices; determining a signal of interest (SOI) that is shared by the plurality of type-specific signatures for the plurality of data types; and determining a sparse linear model of the shared SOI based on the non-zero entries of the plurality of sparse eigenarrays.

In some embodiments, the plurality of distinct data types comprises messenger RNA (mRNA) expression, a microRNA (miRNA) expression, DNA methylation status, single nucleotide polymorphisms (SNPs), next-generation sequencing (NGS) data of entire genomes, next-generation sequencing data of entire transcriptomes, metabolomic data of entire metabolomes, molecular imaging data, or any combination thereof. In some embodiments, the plurality of data types can be collected from a common set of specimens.

In some embodiments, a data type of the plurality of data types comprises measurements of a plurality of variables for a plurality of samples. A data matrix of the plurality of data matrices comprises a plurality of rows and a plurality of columns, wherein the number of the plurality of rows is the number of the plurality of variables, and wherein the number of the plurality of columns is the number of the plurality of samples. The number of the plurality of rows can be greater than the number of the plurality of columns for at least one data type. The method can further comprise pre-processing the measurements of a specific experimental data type into the data matrix, and wherein pre-processing the measurements of the specific experimental data type into the data matrix comprises performing at least one of the following operations or any combination thereof depending on the data type: log 2 transformation, quantile normalization, row-centering, transformations from beta-values to M-values.

In some embodiments, generating the super-matrix comprising the plurality of data matrices comprises stacking the plurality of pre-processed data matrices along columns of the plurality of data matrices and scaling the super-matrix by the Frobenius norm of the super-matrix. In some embodiments, the sparse rank-1 approximation of the super-matrix comprises the best sparse approximation of the super-matrix. The sparse rank-1 approximation of the super-matrix can comprise a converged eigen-array and a converged eigen-signal, wherein the converged eigen-array is sparse wherein the converged eigen-signal is non-sparse and wherein the outer product of the converged eigen-array and the converged eigen-signal can constitute a sparse rank-1 approximation of the super-matrix.

In some embodiments, determining the sparse rank-1 approximation of the super-matrix comprises: (i) determining an initial rank-1 approximation of the super-matrix based on the singular value decomposition (SVD), wherein the initial rank-1 approximation comprises an initial eigen-array and an initial eigen-signal, wherein the initial eigen-array and the initial eigen-signal are non-sparse and wherein the outer product of the initial eigen-array and the initial eigen-signal is an initial non-sparse rank-1 approximation of the super-matrix; (ii) determining a subsequent eigen-array from the initial eigen-array, wherein the subsequent eigen-array is a l₁ regularization of the initial eigen-array, and wherein the subsequent eigen-array is sparse; and (iii) determining a subsequent non-sparse eigen-signal, wherein the outer product of the of the sparse eigen-array and the non-sparse eigen-signal constitutes a sparse rank-1 approximation of the super-matrix.

In some embodiments, the method further comprises (iv) repeating steps (ii) and (iii) until the subsequent eigen-array converges to the converged eigen-array and the subsequent eigen-signal converges to the converged eigen-signal. Repeating steps (ii) and (iii) until the subsequent eigen-array converges to the converged eigen-array and the subsequent eigen-signal converges to the converged eigen-signal in step (iv) can comprise: assigning the subsequent eigen-array as the initial eigen-array; and assigning the subsequent eigen-signal as the initial eigen-signal.

In some embodiments, the sparse multi-modal signature of the super-matrix comprises non-zero elements of the converged eigen-array as the sparse multi-modal signature of the super-matrix. The l₁ regularization of the sparse eigen-arrays subsequent to the initial eigen-array can be based on a false discovery rate. Parsing the sparse rank-1 approximation of the super-matrix to determine the plurality of sparse eigen-arrays for the plurality of data matrices comprises parsing the converged eigen-array of the super-matrix to determine a plurality of sparse eigen-arrays for the plurality of data matrices based on the order of the plurality of data matrices in the super-matrix.

In some embodiments, a rank-1 approximation of a data matrix of the plurality of rank-1 approximations of the plurality of data matrices can be an outer product of a corresponding eigen-array of the plurality of sparse eigen-arrays of the plurality of data matrices and the converged eigen-signal. Parsing the multi-modal signature of the super-matrix to determine the plurality of signatures of the plurality of data matrices can comprise parsing the sparse multi-modal signature of the super-matrix into the plurality of signatures of the plurality of data matrices based on orders of the plurality of data matrices in the super-matrix.

Disclosed herein are systems and methods for encoding a given targeted signature for immune checkpoint blockade (ICB) or another biological process in a machine learning model. In some embodiments, the method comprises receiving data on a plurality of expression patterns associated with a plurality of realizations of a targeted signature determined using a plurality of tissue samples obtained from a plurality of patients with a cancer, wherein the plurality of realizations comprises a realization of the targeted signature for a tissue sample of the plurality of tissue samples of a patient of the plurality of patients, and wherein the realization is associated with an observed outcome determination of a plurality of outcome determinations; generating a plurality of exemplars from the plurality of realizations; and training a machine learning model using the plurality of exemplars.

In some embodiments, the cancer comprises an ovarian cancer or a liver cancer. The plurality of realizations can comprise a matrix of expression measurements of the genes of measured in the plurality of tissue samples. The targeted signature can comprise a plurality of genes that depends on a checkpoint gene and the cancer. The plurality of realizations can comprise a real-valued matrix. The expression patterns of the plurality of realizations can stratify the tissue samples into the plurality of outcome determinations. The plurality of outcome determinations can comprise a good outcome group, a poor outcome group, and an uncertain outcome group.

In some embodiments, the targeted signature comprises a plurality of genes that depends on a checkpoint gene and the cancer. The checkpoint gene can be differentially expressed between the good outcome group and the poor outcome group. The checkpoint gene can be prognostic in a subset of the plurality of patients of the plurality of tissue samples restricted to the good outcome group and the poor outcome group. The targeted signature can be prognostic in the plurality of tissue sample. The realization can be determined using a biochemical assay.

In some embodiments, the machine learning model comprises a classification model. The classification model can comprise a supervised classification model, a semi-supervised classification model, an unsupervised classification model, or a combination thereof. The machine learning model can comprise a neural network, a linear regression model, a logistic regression model, a decision tree, a support vector machine, a Nave Bayes network, a k-nearest neighbors (KNN) model, a k-means model, a random forest model, or any combination thereof.

In some embodiments, the method further comprises receiving data on a plurality of expression patterns associated with a realization of the targeted signature of a second patient and data of the second patient on the cancer; determining a predicted outcome determination of the plurality of outcome determinations using the machine learning model and the realization of the targeted signature of the second patient; determining the predicted outcome determination comprises a good outcome determination; determining the data of the second patient on the cancer is above a threshold; and determining retreatment of the second patient with blockade of a checkpoint gene γ is likely to result in a benefit for the second patient.

Determining the predicted outcome determination comprises a good outcome determination can comprise factoring the plurality of realizations to generate an eigen-survival model (ESM); and projecting the realization of the targeted signature of the second patient into the eigen-survival model to generate a prognostic score for the second patient. The plurality of realizations can be factored using singular value decomposition (SVD) to generate the eigen-survival model (ESM). The prognostic score can comprise an inner product of the eigen-survival model and the realization. Generating the plurality of exemplars from the plurality of realizations can comprise determining a plurality of wavelet coefficients using the realization and the observed outcome determination; filtering the plurality of wavelet coefficients to generate a plurality of filtered wavelet coefficient; and performing singular value decomposition (SVD) of a data matrix of the wavelet coefficients to generate the plurality of exemplars.

In some embodiments, a system comprises computer-readable memory storing executable instructions; and a hardware processor programmed by the executable instructions to perform any of the methods disclosed herein. The system can further comprise a data store configured to store the multi-modal data sets, the sparse multi-modal signature, and the plurality of sparse rank-1 approximations of the plurality of data matrices. In some embodiments, a computer readable medium comprises a software program that comprises codes for performing any of the method disclosed herein.

### BRIEF DESCRIPTION OF THE DRAWINGS

FIG. 1 compares an exemplary classical data matrix and an exemplary big data matrix.

FIG. 2 shows an exemplary 20kx61 expression data matrix for liver cancer.

FIG. 3 shows schematic illustrations of the singular value decomposition (SVD) of a data matrix.

FIG. 4 shows an exemplary model as a weighted sum of the rows of the data matrix.

FIG. 5 shows that the rank-1 model of V₁ in FIG. 4 has dimensionality of 20,792.

FIG. 6 shows an exemplary JAMMIT workflow.

FIG. 7 shows an exemplary output of JAMMIT in the AWS cloud.

FIG. 8 shows an exemplary output of parallelized JAMMIT in the AWS cloud.

FIG. 9 shows an exemplary JAMMIT workflow of global mRNA, microRNA, and methylation data from 291 ovarian tumors from TCGA.

FIG. 10 shows a flowchart of an exemplary JAMMIT optimization algorithm.

FIG. 11 shows an exemplary workflow for identification of good responders using JAMMIT.

FIG. 12 depicts a general architecture of an example computing device configured to perform joint analysis of multiple high-dimensional data types using sparse matrix approximations of rank-1.

FIG. 13 shows example distributions of ΔAUROC values comparing JAMMIT detection performance with two other algorithms in simulated data.

FIG. 14 shows exemplary plots showing mRNA detector U₁ and signal of interest V₁ for ovarian cancer.

FIG. 15 shows clustered heatmaps of sparse signatures for ovarian cancer discovered by JAMMIT. FIG. 15, panel (A) shows an exemplary heatmap of mRNA signature with one of three distinct meta-variables highlighted in yellow. FIG. 15, panel (B) shows an exemplary heatmap of microRNA signature with two coherent meta-variables highlighted in green and yellow. FIG. 15, panel (C) shows an exemplary heatmap of methylation signature with one of two distinct meta-variables highlighted in yellow. FIG. 15, panel (D) shows a heatmap of joint mRNA+methylation signature with one of four distinct meta-variables highlighted in green.

FIG. 16 shows plots of eigen-survival analysis of JAMMIT multimodal signature composed of mRNA and methylation variables for 291 patients. FIG. 16, panel (A) shows exemplary KM plots of based on MMSIG composed of mRNA and methylation variables. FIG. 16, panel (B) shows exemplary KM plots based on signature composed mRNA variables only. FIG. 16, panel (C) shows exemplary KM plots based on signature composed of methylation variables only. Note the superior p-values, median survival time and 5-year survival rate for the signature that combines variables for the mRNA and microRNA data types.

FIG. 17 shows 40-gene signature for ovarian cancer anchored upstream by IL4 is robustly associated with survival. FIG. 17, panel (A) shows an exemplary clustered heatmap of the mRNA signature φ_{IL4}⁽⁴⁰⁾ realized in the 291-sample training data set. FIG. 17, panel (B) shows exemplary KM plots of patients in training data set with prognostic scores in the top and bottom quartiles based on the eigen-survival model based on the realization of φ_{IL4}⁽⁴⁰⁾ in 291-sample discovery data set. FIG. 17, panel (C) shows an exemplary clustered heatmap of φ_{IL4}⁽⁴⁰⁾ realized in the 99-sample test data set. FIG. 17, panel (D) shows an exemplary KM plots of patients in unseen test data set with prognostic scores in the top and bottom quartiles. The prognostic scores for the test patients were obtained by projecting the realization of φ_{IL4}⁽⁴⁰⁾ in the test data onto the eigen-survival model for φ_{IL4}⁽⁴⁰⁾ derived from the discovery data set (green arrows).

FIG. 18 shows exemplary loading coefficients of eigen-survival model derived from φ_{IL4}⁽⁴⁰⁾ in the 291-sample discovery data set. Genes that contribute most significantly to the eigen-survival model derived from the 291-sample discovery data set are highlighted by red squares. These genes were used to define a 12-gene mRNA signature φ_{IL4}⁽⁴⁰⁾ that was evaluated for association with overall survival and biological coherence.

FIG. 19 shows an exemplary 12-gene mRNA signature for ovarian cancer anchored upstream by IL4 predicts overall survival. FIG. 19, panel (A) shows exemplary KM plots of patients in discovery data set with prognostic scores based on the 12-gene mRNA signature φ_{IL4}⁽¹²⁾ in the top (red) and bottom (blue) quartiles. Note the two groups of 72 patients each (144 total) show significant differences in survival based on the separation between their respective KM plots. FIG. 19, panel (B) shows exemplary KM plots of patients in test data set with φ_{IL4}⁽¹²⁾ prognostic scores in the top (red) and bottom (blue) quartiles. The two groups of 24 patients each (48 total) show significant differences in survival based on the separation between their respective KM plots. The prognostic scores for the test patients were computed by projecting the test data matrix for φ_{IL4}⁽¹²⁾ onto the ESM derived from discovery data matrix for φ_{IL4}⁽¹²⁾.

FIG. 20 illustrates that immune checkpoint genes are differentially expressed between good and poor responders to chemotherapy per the IL4 signature.

FIG. 21 outlines JAMMIT analysis of whole-genome mRNA and PET imaging data for liver cancer.

FIG. 22 shows an exemplary clustered heatmap of the K₁/k₂ signature identified by JAMMIT in 50 liver tissue samples. The heatmap for the K₁/k₂ signature, ω_{mRNA}^{(K}^{₁}^{/k}^{₂}⁾, exhibits very uniform expression on the normals (columns 1-20) and very high variability on the tumor samples. On the tumor samples, significant down-regulation of ω_{mRNA}^{(K}^{₁}^{/k}^{₂}⁾ expression patterns on a subset of seven (7) HCC, six (6) ICC and 2 sarcomas was observed. The remaining 15 HCC had ω_{mRNA}^{(K}^{₁}^{/k}^{₂}⁾ expression profiles very similar to the 20 normal samples.

FIG. 23 shows results of cluster analysis by the K₁/k₂ signature reveals a novel subtype of HCC metabolically similar to ICC. FIG. 23, panel (A) shows an exemplar 2-way hierarchically clustered heatmap of K₁/k₂ signature in the 50-sample discovery data set. This analysis identified two distinct expression phenotypes Γ⁽⁻⁾ and Γ⁽⁻⁾ where included samples where ω_{mRNA}^{(K}^{₁}^{/k}^{₂}⁾ were down-regulated on the samples in Γ⁽⁻⁾ relative to the remaining samples in Γ⁽⁺⁾. The Γ⁽⁻⁾ class contained all 6 ICC samples plus 7 HCC and 2 sarcomas while Γ⁽⁺⁾ contained all 20 normal samples along with 15 HCC. FIG. 23, panel (B) shows an exemplary plot of the dominant eigen-signal of the matrix for the ω_{mRNA}^{(K}^{₁}^{/k}^{₂}⁾ signature clearly separates the samples in Γ⁽⁻⁾ and Γ⁽⁺⁾ based on a threshold set at zero.

FIG. 24 shows the ABCB11 gene discriminates between the Γ⁽⁻⁾ and Γ⁽⁻⁾ expression phenotypes. FIG. 24, panel (A) shows ABCB11 expression over 6 ICC (columns 1-6) and 22 HCC (columns 7-28). FIG. 24, panel (B) shows ABCB11 expression over 20 normals (columns 1-20) and 30 tumors (columns 21-50).

FIG. 25 shows exemplary expression profiles of selected nuclear receptors and transporter genes associated with the K₁/k₂ liver signature. Shown are normalized expression profiles of selected genes associated with the K₁/k₂ signature in two experimental designs denoted by ICC vs HCC and NRM vs TUMOR. Each lettered panel contains top and bottom sub-panels showing the profile of a gene in the ICC vs HCC and NRM vs TUMOR designs, respectively. In the top panels, columns 1-6 represent ICC samples and columns 7-28 HCC samples, while in bottom sub-panels, columns 1-20 represent normal samples and columns 21-50 represent liver tumors (6 ICC, 2 sarcomas and 22 HCC). Red squares represent ICC samples, green triangles represent CCL-HCC samples, and blue circles represent normal and HCC samples. FIG. 25, panel (A) top panel shows FXR is down-regulated on ICC (cols 1-6) relative to HCC while the bottom panel shows that FXR is uniformly up-regulated on the normals and preferentially down-regulated on a subset of tumors that includes 6 ICC and 2 of 7 CCL-HCC. FIG. 25, panel (B) shows that HNF4A expression patterns to be similar to FXR over the two groupings of the samples, i.e., preferential down-regulation on the ICC and CCL-HCC relative to the normals and HCCs. FIG. 25, panel (C) shows SLC2A1/GLUT1 is a transporter that is negatively correlated with the K₁/k₂ PET parameter and preferentially up-regulated on the ICC and CCL-HCC samples relative to the normal and HCC samples. FIG. 25, panel (D) shows that SLC6A14 is strikingly up-regulated on all 6 ICC samples and less so on 5 of 7 CCL-HCC samples relative to the normal and HCC samples.

FIG. 26 shows identification of an 11-gene super-signature for liver cancer.

FIG. 27 shows up-regulation of bad hepatocellular carcinomas (HCCs) from 32 patients.

FIG. 28 shows that fatty acid uptake genes were down-regulated in Fatty-Warburg HCCs.

FIG. 29 shows discriminating between two expression phenotypes based on the PET kinetic parameter K₁/k₂. Points in scatter plots represent output of Generalized Regression Neural Networks (GRNNs) trained to discriminate between two expression phenotypes denoted by Γ⁽⁻⁾ and Γ⁽⁺⁾ identified by the ω_{mRNA}^{(K}^{₁}^{/k}^{₂}⁾ expression signature. Expression phenotype Γ⁽⁻⁾ contains 7 HCC, 6 ICC and 2 sarcomas while phenotype Γ⁽⁺⁾ contains 20 normals and 25 HCC. In each panel, columns 1-20 represent normals and columns 21-50 represent liver tumors (15 HCC, 6 ICC, 2 sarcomas, 7 CCL-HCC). Horizontal line (magenta) represents a threshold τ on the GRNN output where samples with GRNN values greater than τ are assigned to Γ⁽⁻⁾, otherwise the sample is assigned to Γ⁽⁺⁾. FIG. 29, panel (A) GRNN output based on K₁/k₂ parameter vector aligned with sample grouping described herein. Note that all members of Γ⁽⁻⁾ and all but one of the normal samples are correctly classified with some confusion on the HCC samples with correct classification rate of 87%. FIG. 29, panel (B) GRNN output on a random permutation of the K₁/k₂ parameter vector showing poor overall classification performance. Only 1 out of 1000 permutations of the K₁/k₂ parameter vector had a correct classification rate greater than 86%, which resulted in an empirical p-value of 0.001 for the observed classification pattern in panel (A).

FIG. 30 shows an example workflow for sparse signal processing of multi-modal data.

FIG. 31 shows an example workflow used to determine multi-modal cancer signatures that were predictive of clinical outcome.

FIG. 32 is a non-limiting exemplary plot showing that an aggressive tumor subtype was identified by the 4-gene glycolytic signature.

FIG. 33, panels (A)-(D) are plots showing that the 4-gene signature was used to identify a subset of patients who may derive a survival benefit from TIM-3 blockade.

FIG. 34 shows an example JAMMIT workflow for analysis of ovarian cancer.

FIG. 35 shows that IL4Sig40 identifies a subset of patients who may derive a survival benefit from PD-L1 blockade.

### DETAILED DESCRIPTION

In the following detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments may be utilized, and other changes may be made, without departing from the spirit or scope of the subject matter presented herein. It will be readily understood that the aspects of the present disclosure, as generally described herein, and illustrated in the Figures, can be arranged, substituted, combined, separated, and designed in a wide variety of different configurations, all of which are explicitly contemplated herein and made part of the disclosure herein.

All patents, published patent applications, other publications, and sequences from GenBank, and other databases referred to herein are incorporated by reference in their entirety with respect to the related technology.

### Definitions

Unless defined otherwise, technical and scientific terms used herein have the same meaning as commonly understood by one of ordinary skill in the art to which the present disclosure belongs. See, e.g. Singleton et al., Dictionary of Microbiology and Molecular Biology 2nd ed., J. Wiley & Sons (New York, N.Y. 1994); Sambrook et al., Molecular Cloning, A Laboratory Manual, Cold Springs Harbor Press (Cold Springs Harbor, N Y 1989). For purposes of the present disclosure, the following terms are defined below.

### JAMMIT Overview

Technological advances enable the cost-effective acquisition of multi-modal data sets (MMDS) composed of multiple, high-dimensional data matrices that represent the measurements of multiple data types obtained from a common set of samples. In some embodiments, the joint analysis of the multiple data matrices of a MMDS can provide a more focused view of the biology underlying cancer and other diseases that would not be apparent from the analysis of a single data type in isolation. Multi-modal data are rapidly accumulating in research laboratories and public databases such as The Cancer Genome Atlas (TCGA). The translation of such data into clinically actionable knowledge is slowed by the lack of computational tools capable of jointly analyzing the data matrices of a given MMDS. Disclosed herein is the Joint Analysis of Many Matrices by ITeration (JAMMIT) algorithm for the analysis of MMDS using sparse matrix approximations of rank-1.

Data Compression: The essence of science can be to reduce lists of observations into an abbreviated form based on the recognition of patterns (i.e., signals) of low dimensionality, i.e., data compression. Such signals enable the original observations to be replaced by a short-hand formula, or model, which accurately predicts the future. “Big” data sets composed of many measurements per observation that contain low-dimensional signals are in principle compressible. A random sequence may not compressible since there is no low-dimensional model that can predict future values of the sequence. The sequence of even integers is highly compressible since a short computer program can generate any even integer (algorithmic compressibility). All data collected or will ever be collected about the motion of bodies in the heavens and on Earth can be compressed into Newton's three laws of motion and universal gravitation.

Sparsity and compressibility. Low-dimensional signals embedded in high-dimensional data can be referred to as sparse signals. Data that contain sparse signals may be highly compressible. Almost all signals can be sparsified by an appropriate transformation (e.g., Fourier, wavelets, etc.). Most images are compressible because edges are sparse and contain information tuned for the human visual system (JPEG algorithm based on wavelets). Big genomic data sets can be highly compressible since they contain sparse signals.

Sparsity and genomics. Biologically important signals in big genomic data can be sparse. For example, only a small percentage of the many thousands of genes interrogated by a DNA microarray are needed to characterize differences between tumor and normal cells. As another example, only a small percentage of over 400,000 methylation loci measured on current platforms are needed to predict response to treatment. As a further example, detecting a sparse subset of relevant variables in a big data set is like finding a bunch of needles in a haystack. In some embodiments, methods that exploit signal sparsity can better identify biologically and/or clinically relevant signals in genomic data.

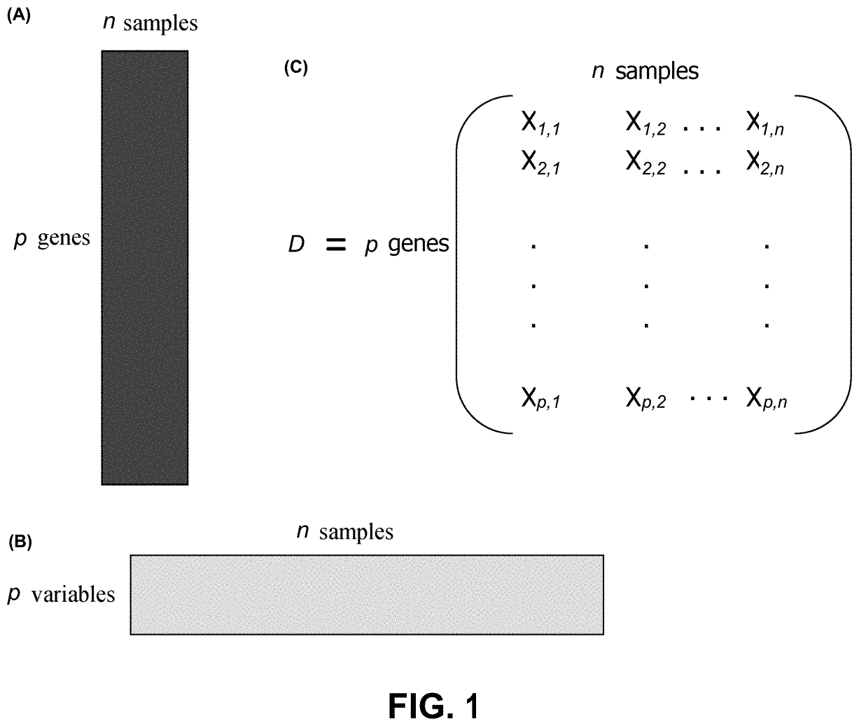

Data Matrices. FIG. 1, panel (A) shows an exemplary classical data matrix, which is short and wide (p<<n, where p denotes the number of variables and n denotes the number of samples). The data matrix has many more equations than unknowns. Classical statistics apply, i.e., systems of linear equations, least squares, law of large numbers, etc. And n can be taken to infinity to obtain optimal estimates of relatively small number of P variables.

FIG. 1, panel (B) shows an exemplary big data matrix which is tall and thin (p>>n, where p denotes the number of variables and n denotes the number of samples). There are fewer equations than unknowns. Standard methods fail, e.g., infinite number of solutions, multiple comparisons problem, sparse signal, low SNR and lots of noise and clutter, etc. Mathematical theories that leverages high p and low n (e.g., compressive sensing, LASSO, wavelet transformation, etc.) are lacking.

FIG. 1, panel (C) shows an example data matrix D, with p genes and n samples, where p>>n. Xᵢⱼ denotes the ith gene in jth sample, where i=1, 2, . . . , p and j=1, 2, . . . , n. In some embodiments, the methods disclosed herein determine a number of rows that explain a dominant signal of interest (SOI) over n samples contained in the data matrix D. The collection of the genes determined can be referred to as a signature of the SOI.

The JAMMIT algorithm jointly approximates an arbitrary number of data matrices by rank-1 outer-products composed of “sparse” left-singular vectors (eigen-arrays) that are unique to each matrix and a right-singular vector (eigen-signal) that is common to all the matrices. The non-zero coefficients of the eigen-arrays select small sets of variables for each data type (i.e., signatures) that in aggregate, or individually, best explain the common eigen-signal shared by all the data matrices. The approximation is specified by a single “sparsity” parameter that is selected based on false discovery rate estimated by permutation testing. Multiple signals of interest in a given MMDS are sequentially detected and modeled by iterating JAMMIT on residual data matrices that result from a given approximation.

In some implementations, that JAMMIT outperforms other joint analysis algorithms on simulated MMDS. In some embodiments, on real multimodal data for diseases (e.g., ovarian and liver cancer), JAMMIT can enable the discovery of low-dimensional, multimodal signatures that were clinically informative and enriched for cancer-related biology.

In some embodiments, sparse matrix approximations of rank-1 are an effective means of jointly reducing multiple, big data types obtained from a common set of bio-samples to low-dimensional signatures composed of variables of different types that characterize sample attributes of potential clinical and biological significance.

Workflow Overview

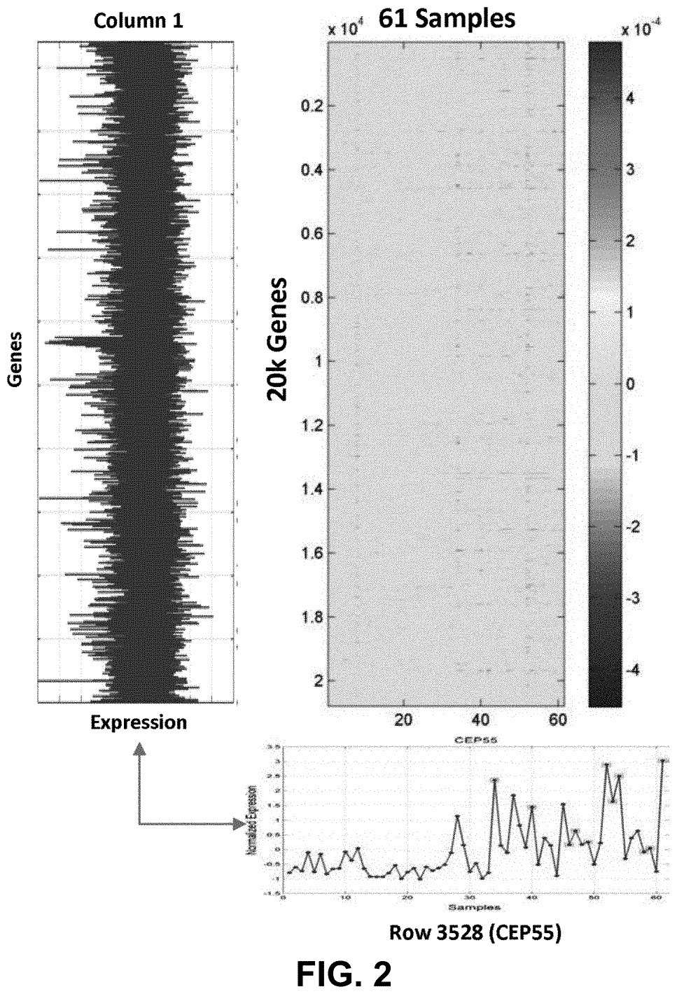

FIG. 2 shows an exemplary 20kx61 expression data matrix for liver cancer. The exemplary matrix includes 20792 genes for each of 61 samples. Columns 1-33 represent normal tissue. Columns 34-61 represent hepatocellular carcinoma. In some embodiments, the data matrix can be pre-processed. Pre-processing of the data matrix can include one or more or: generalized log 2 transform, background subtraction, quantile normalization, Frobenius scaling, and row centering.

SVD of Data Matrix.

Let D=U*S*V^{T} be the singular value decomposition (SVD) of D. In some embodiments, generalizes principal components analysis (PCA) from n×n matrices to p×n matrices. This can enable analysis of interactions between the rows (e.g., genes) and columns (e.g., samples) of D. Thus, SVD can be more numerically stable than PCA, which can be important when analyzing “big” data. In some embodiments, D≈U₁*S₁*V₁^{T}, the best rank-1 approximation of D (least squares sense) where U₁ and V₁ are the top eigen-vectors of D that correspond to the 1st columns of U and V, respectively, and Si is the top singular value of D. It follows that V₁=D^{T}*U₁*(1/Si). The signal on n samples represented by V₁ can be modeled as the sum of all p rows of D with weights from U₁. The linear model of V₁ can be based on weights from U₁ is p dimensional, i.e., it may be difficult to interpret and understand.

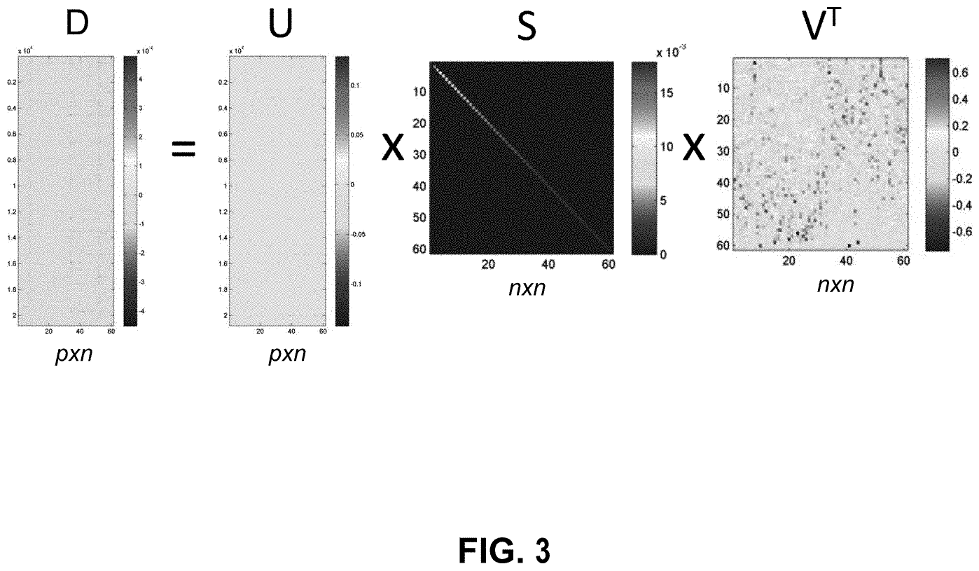

FIG. 3 shows schematic illustrations of the singular value decomposition (SVD) of a data matrix. As shown in FIG. 3, SVD stratifies the variation in D in n orthogonal directions. Columns of U (eigen-arrays) stratify variation over the rows of D in n orthogonal directions. Columns of V (eigen-genes) stratify variation over the columns of D inn orthogonal directions. Diagonal elements of S (singular values) represent the variance in each orthogonal direction. The columns of U and V are ordered (left-to-right) by singular values.

Best Rankd-1 Approximation.

The best rank-1 approximation of the data matrix D=U*S*V^{T}=ΣUₖ*Sₖ*Vₖ^{T}, k=1 to n=U₁*S₁*V₁^{T}+U₂*S₂*V₂^{T}+ . . . +Uₙ*Sₙ*Vₙ^{T}, where Uₖ denotes kth column of 1i=loadings for kth orthogonal signal, the loading of the kth orthogonal signal, Sₖ denotes kth diagonal of S, the variance for kth orthogonal signal, and Vₖ denotes kth column of V, the scores for kth orthogonal signal. D≈U₁*S₁*V₁^{T}, the best rank-1 approximation of D.

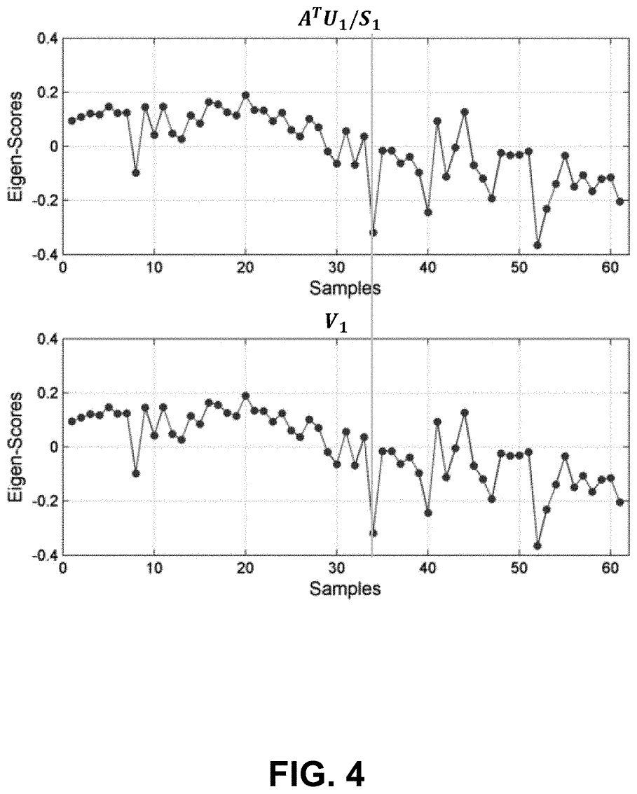

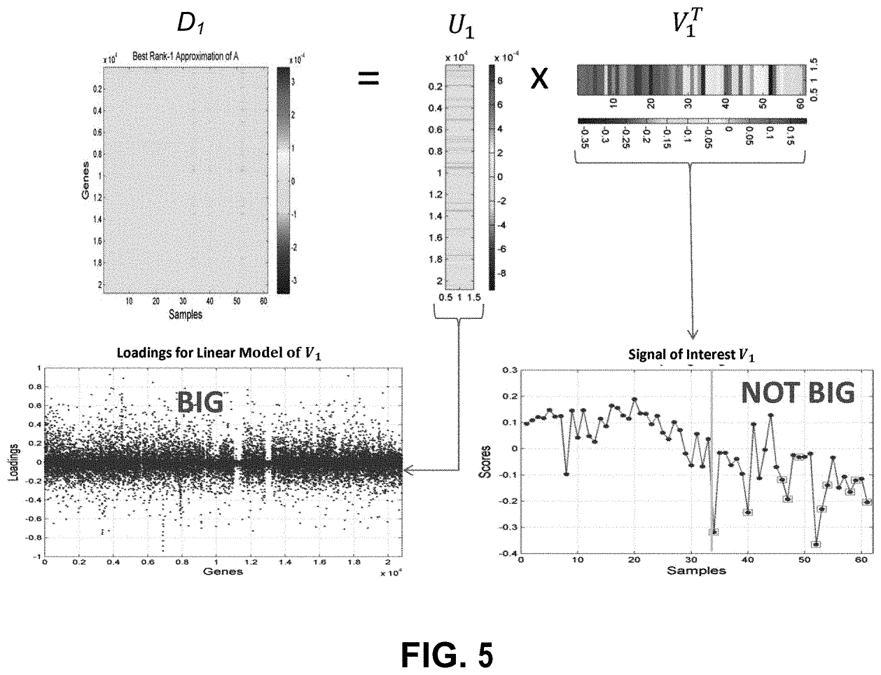

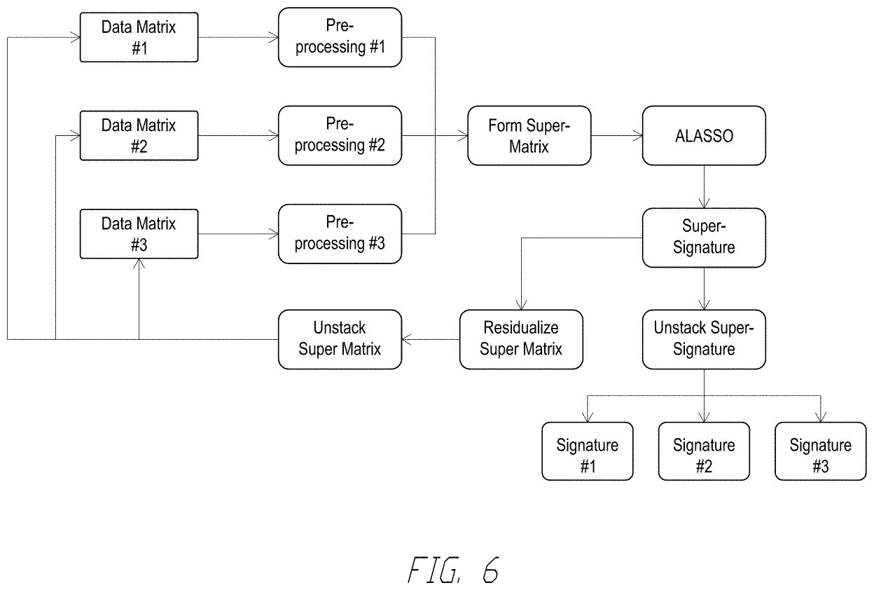

The SVD of D allows the linear modeling of V₁ as a weighted sum of the P rows of D with weights from U because V₁=(A^{T}U₁/S₁). FIG. 4 shows an exemplary model of V₁ as a weighted sum of the P rows of D with weights from U. FIG. 5 shows that the rank-1 model of V₁ has dimensionality of 20,792.

The Asymmetric LASSO (ALASSO). In some embodiments, the methods disclosed herein can “sparsify” U₁ (but not V₁) based on the application of an asymmetric version of the Least Absolute Shrinkage and Selection Operator (ALASSO) to rank-1 matrix factorizations. ALASSO can uses the l1-norm to shrink all but a sparse subset of p (p<<P) coefficients of U₁ to zero while constraining the final solution to be a rank-1 approximation of D. If the important variables are truly sparse in U₁, then ALASSO will find them and resulting rank-1 model of V1 will have dimension p where p<<P. Otherwise, no algorithm will help unless more samples are obtained (Bellman's Curse of Dimensionality).

Data Fusion.

In some embodiments, two or more data matrices associated with different genomic data types acquired from a common set of bio-samples are analyzed. For example, the data matrices can include mRNA, microRNA, DNA methylation, SNPs, and mutation data for over 30 different cancers in The Cancer Genome Atlas (TCGA). As another example, the data matrices can include mRNA, metabolomic, and PET/CT imaging data collected for a collection of normal and tumor samples in liver cancer. The kth data type can be assumed to generate a Pₖ×N data matrix for k=1, 2, . . . , K where Pₖ>>N for at least one k. The methods can obtain a small set of variables (i.e., signature) for each data type that individually or in combination predicts the clinical trajectory of cancer (sparse data fusion).

Joint Analysis of Many Matrices by ITeration (JAMMIT) for Sparse Data Fusion.

The methods disclosed herein can pre-process each data matrix separately. In some embodiments, the methods can vertically stack the data matrices along their N columns to form a super-matrix, scale the super-matrix by its Frobenius norm, and center the rows of the scaled super-matrix. The methods can apply the ALASSO to the centered and scaled super-matrix to compute a super-signature composed of a small number of variables for each data type and deconstruct the super-signature into sparse, type-specific signatures.

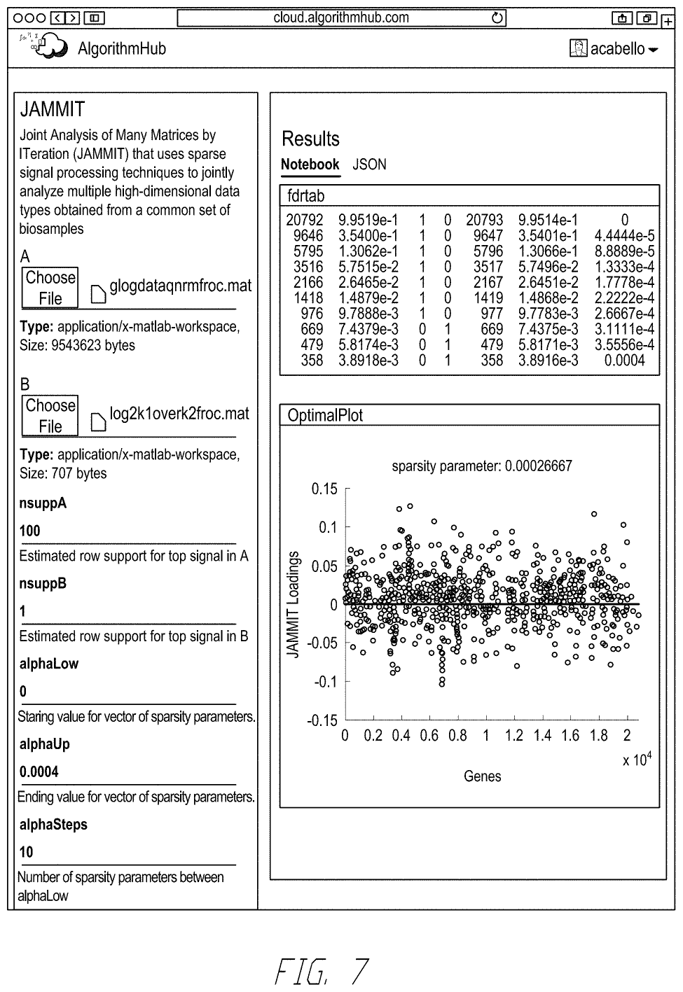

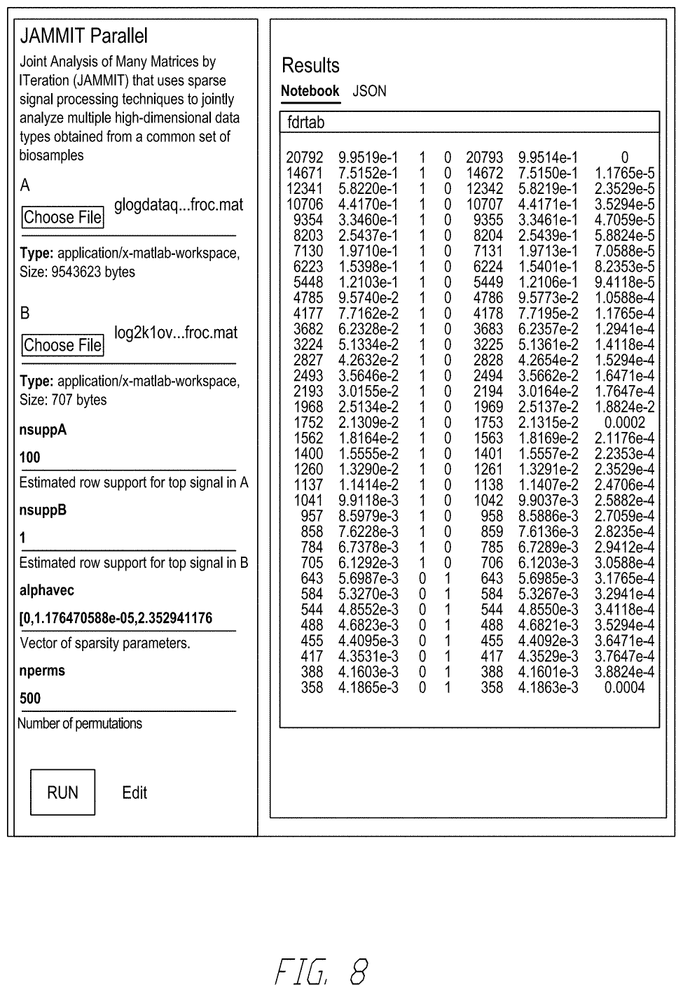

FIG. 6 shows an example JAMMIT Workflow. In some embodiments, the JAMMIT workflow can select an optimal sparsity parameter A based on False Discovery Rate (FDR). For example, FDR can be computed on a monotonically increasing grid of)'s where each FDR is estimated based on a large number of permutations. The optimal parameter can be selected based on FDR value and size of super- and type-specific signatures. In some embodiments, such computations can be computationally intensive. In some embodiments, such computations can take 8 hours for 500 permutations on 35 parameters. In some embodiments, JAMMIT can be implemented on Amazon Web Services (AWS), which can compute FDR table based on 500 permutations on 35λ's in less than 8 minutes. FIG. 7 shows an exemplary output of JAMMIT in the AWS cloud. FIG. 8 shows an exemplary output of parallelized JAMMIT in the AWS cloud.

Data Matrix

Advances in array technology, high-throughput sequencing, and clinical imaging platforms enable the measurement of tens of thousands of variables of a specific data type in a fixed set of tissue samples. Examples of such “big” data types include genome-wide measurements of messenger RNA (mRNA) and microRNA (miRNA) expression, DNA methylation status, single nucleotide polymorphisms (SNPs), next-generation sequence data of entire genomes and transcriptomes, and features extracted from molecular imaging platforms.

Measurements of the p>1 variables of a given data type obtained from a collection of n>1 samples can be organized into a p×n data matrix D with rows representing variables and columns representing measurements of the p variables in each of the n samples. In some embodiments, the method selects s>0 rows of D that best approximate a dominant Signal of Interest (SOI) in the row-space of D since such signals could represent a sample attribute of clinical and/or biological significance. For big data types, D will have many more rows (variables) than columns (samples), making such “tall and thin” data matrices difficult to analyze using standard statistical techniques due to a severe multiple comparisons problem and low signal-to-noise ratio (SNR). The low SNR is due in large part to the relatively small number of variables (out of many thousands measured) that are truly associated with a given biological and/or clinical attribute of the samples. This small subset of significant variables constitutes a “sparse signature” in the set of all measurements represented by D in the sense that s<<p.

Multi-Modal Data Sets (MMDS) composed of multiple data matrices of two or more distinct data types obtained from a common set of bio-samples pose even greater analytical challenges if the goal is to jointly analyze the data matrices in an integrated manner, which exacerbates problems related to data dimensionality and SNR. As before, the goal is to identify sparse signatures for each data type that individually, or in combination, explain a SOI that characterizes an important biological and/or clinical attribute of the samples. Unfortunately, the lack of analytical tools for the joint analysis of multiple data types has impeded the discovery of novel predictive biomarkers and therapeutic targets that account for interactions between networks of diverse molecular species across space and time. Moreover, MMDS are accumulating at an exponential rate in academic research laboratories, private industry, and public data repositories such as The Cancer Genome Atlas (TCGA) and the International Cancer Genome Consortium (ICGC) as the per sample cost of data acquisition plummets. This growing inventory of MMDS presents a major analytical bottleneck in the translation of big, multimodal data into clinically actionable knowledge.

In some embodiments, the measurements for K>1 different data types collected from a common set of n biospecimens ζₙ={ζ₁, ζ₂, . . . , ζₙ}, can be represented by a collection of K matrices, [symbol] ={Dₖ}ₖ₌₁^{K}, such that: Dₖ is the pₖ×n data matrix representing measurements for the kth data type; and at least one of the Dₖ is big i.e., pₖ>>n. In some embodiments, each Dₖ can be assumed to have been appropriately pre-processed as function of its data type. For example, pre-processing of mRNA data would involve log 2-transformation, quantile normalization, and row-centering, while a methylation data matrix would be transformed from Beta-values to M-values prior to normalization and row-centering. Let D=D( [symbol] ) be the p×n super-matrix that vertically “stacks” each of the pre-processed pₖ×n matrices Dₖ∈ [symbol] along their columns, where Σₖ₌₁^{K}pₖ. D can be assumed to be appropriately scaled by its Frobenius norm to account for differences in the number of rows and dynamic range of the different Dₖ's. Then the joint analysis of D involves the identification of s>0 rows of the super-matrix D that models a univariate SOI in the row-space of D as a linear combination of the selected rows. The set of s variables associated with the selected rows define a multi-modal signature (MMSIG) of D denoted by ζ such that s=dim(ζ) If the SOI is highly correlated with an important biological and/or clinical attribute of the samples, then ζ helps to explain and interpret the sample attribute of interest in terms of the selected variables. Note that since D is big (i.e., p>>n), it may be desirable for to be sparse in D, (i.e., s<<p) to facilitate downstream interpretation and validation of the SOI.

Matrix approximations of rank-1 provide an efficient way of jointly analyzing the matrices of [symbol] . For example, assume the super-matrix D has rank R>0 and let D=Σᵣ₌₁^{R}uᵣσᵣvᵣ^{T} be the singular value decomposition (SVD) of D where: uᵣ∈ [symbol] ^{P} is the rth left-singular vector (i.e., the rth singular value for i=1, 2, . . . , R). Then the outer-product, u₁σ₁v₁^{T} is the best rank-r approximation of D in at least squares sense and v₁ represents the dominant SOI on the columns of D that is linearly modeled in terms of the p rows of D weighted by the “loading coefficients of u₁. If D is big, then dim(ζ_{SVD})=p is large since the SVD in general assigns a non-zero loading to each row of D, which poses problems for downstream validation and interpretation of v₁ in terms of the p variables of ζ_{SVD}.

Instead, the Bet On Sparsity (BEST) principle is applied that states that if p>>n, then it is best to assume that v₁ is sparsely supported by a small number of rows of D, and employ an l₁ penalty to identify these rows. If the sparsity assumption is true then v₁ will be optimally modeled by the selected rows; otherwise no method will be able to recover the underlying model without many more samples (i.e., Bellman's curse of dimensionality.) Taking the BEST approach, the Joint analysis of Many Matrices by Iteration (JAMMIT) algorithm can be used to approximate D by the rank-1 outer-product, D≈uv^{T}, where u∈uᵣ∈ [symbol] ^{P} is a sparse, eigen-array of “loading” coefficients and v∈uᵣ∈ [symbol] ^{P} is non-sparse, eigen-signal of “scores” that potentially represents an important biological and/or clinical attribute of the samples. The algorithm uses an “asymmetric” version of the Least Absolute Shrinkage and Selection Operator (LASSO) that regularizes u but not v as a function of a l₁ penalty selected based on false discovery rate (FDR).

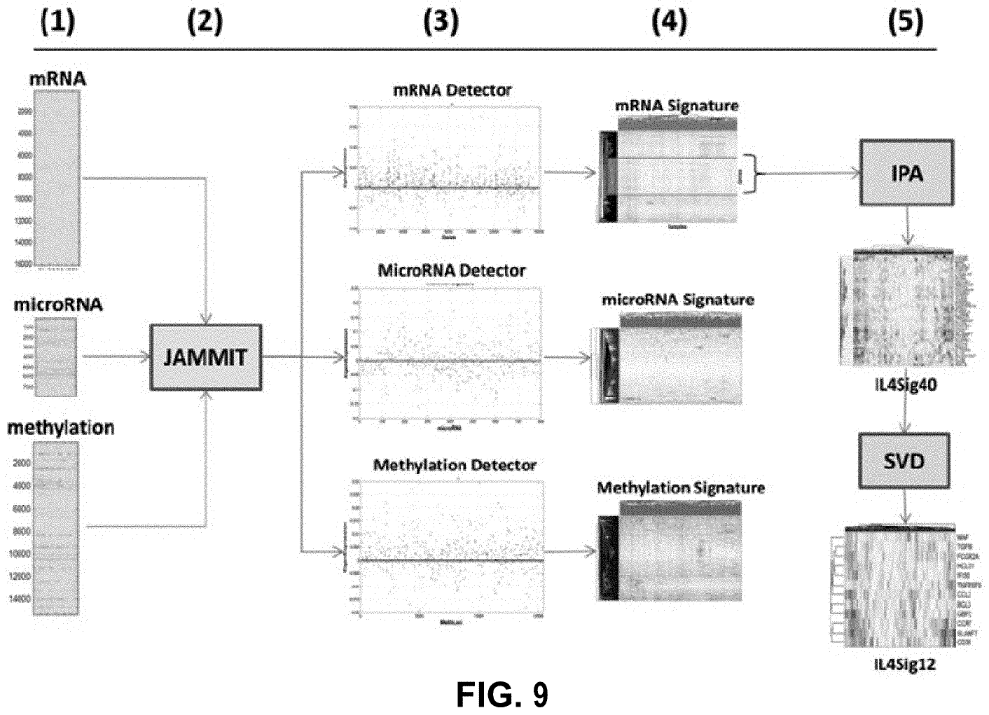

The small number of non-zero coefficients of define a sparse MMSIG in that supports an s-dimensional, linear model such that s<<p. Since a given MMDS is likely to contain multiple SOIs of biological and/or clinical relevance, the JAMMIT algorithm is iteratively applied to the residuals of the current model to identify and model any additional SOI in the data (see Methods under The JAMMIT algorithm for more details). FIG. 9 shows an exemplary JAMMIT workflow of three big data types for ovarian cancer downloaded from TCGA. The workflow in FIG. 9 focuses on iteration #1 of a JAMMIT analysis of a MMDS composed of three large data matrices that was reduced in a step-wise fashion to a 12-gene signature (see Results for more details). This mRNA signature was found to be predictive of overall survival and enriched for biology associated with immunological response in the tumor microenvironment.

Step (1) Heat maps of mRNA, microRNA and DNA methylation data matrices assembled and pre-processed for input to JAMMIT algorithm. Step (2) JAMMIT analysis with minus-one cross-validation. Step (3) Scatter plots of sparse eigen-arrays generated by JAMMIT for each data type. Note that most of the variables for each data type have zero weighting. Step (4) 2-way hierarchically clustered heatmaps of each type-specific signature selected by the non-zeros coefficients of the corresponding sparse eigen-array. Note each heatmap enables the visual identification and extraction of coherent “metavariables” composed of type-specific variables that exhibit coordinated patterns of variation. Step (5) the mRNA meta-variable signature is further reduced using IPA and the SVD to arrive at a 12-gene expression signature that was regulated upstream by IL4. Subsequent eigen-survival and pathway analysis of the 12-gene signature established a connection between overall survival of patients with stage 3 disease being treated with platinum-based chemotherapy plus taxane and the distribution of the M1 and M2 macrophage phenotypes in the tumor micro-environment.

In some embodiments, the information processing flows from left to right in five steps illustrating how three large data matrices are reduced to three relatively small type-specific signatures shown in step 4. Also illustrated is post-JAMMIT processing involving pathway and matrix analysis that is necessary to further reduce signature dimensionality without the loss of critical information, which eventually results in a 12-gene mRNA signature that connects overall survival and immune response in the tumor microenvironment.

Other methods based on matrix factorizations have been proposed for the joint analysis of multiple data types such as the Generalized Singular Value Decomposition (GSVD), Joint and Individual Variation Explained (JIVE), DISCO-SCA, Partial Least Squares (PLS), and Canonical Correlation Analysis (CCA). These methods suffer from the same problem as the SVD in that they minimize the l₂ norm of the estimation error and assign non-zero weights to all p rows of D. A number of techniques can be used to reduce the dimensionality of the selected model such as: rotation of principal components as implemented in factor analysis; ignoring loadings smaller than some threshold; and restricting the range of the loadings to a small discrete set of values. Unfortunately, these methods are prone to high false positive rates and poor sensitivity especially in situations where the SNR is low. Regularized versions of Principal Components Analysis (PCA), SVD, CCA and PLS have been proposed for sparse signal detection and dimensionality reduction, but application of these methods to the super-matrix that “stacks” an arbitrary number of data matrices is not explicitly discussed. Finally, many of the methods outlined herein focus on maximal rank-k approximations of D where k is significantly greater than one, which precludes the use of resampling methods in the selection of the best l₁ penalty due to the high computational cost.

Disclosed herein is an exemplary workflow for the joint analysis of multiple data types based on the JAMMIT algorithm. The disclosure provides technical details on the algorithm and the computational tools used to evaluate the statistical significance, biological coherence, and clinical relevance of JAMMIT-derived signatures. In some embodiments, the JAMMIT detection performance can be superior to that of other joint analysis algorithms on simulated data. For real experimental data, a JAMMIT analysis of global mRNA, microRNA and DNA methylation data for ovarian cancer down-loaded from TCGA resulted in immunological signatures that were predictive of overall survival in the context of platinum-based chemotherapy. In some embodiments, a JAMMIT analysis of whole-genome mRNA data for a disease (e.g., liver cancer) was supervised by quantitative features derived from Positron Emission Tomography (PET) imaging data, which resulted in the discovery of a novel subtype of hepatocellular carcinoma (HCC) that was characterized by elevated aerobic glycolysis (Warburg Effect), suppressed lipid and bile acid metabolism, and poor prognosis.

The Joint Analysis of Many Matrices Via ITeration (JAMMIT) Algorithm

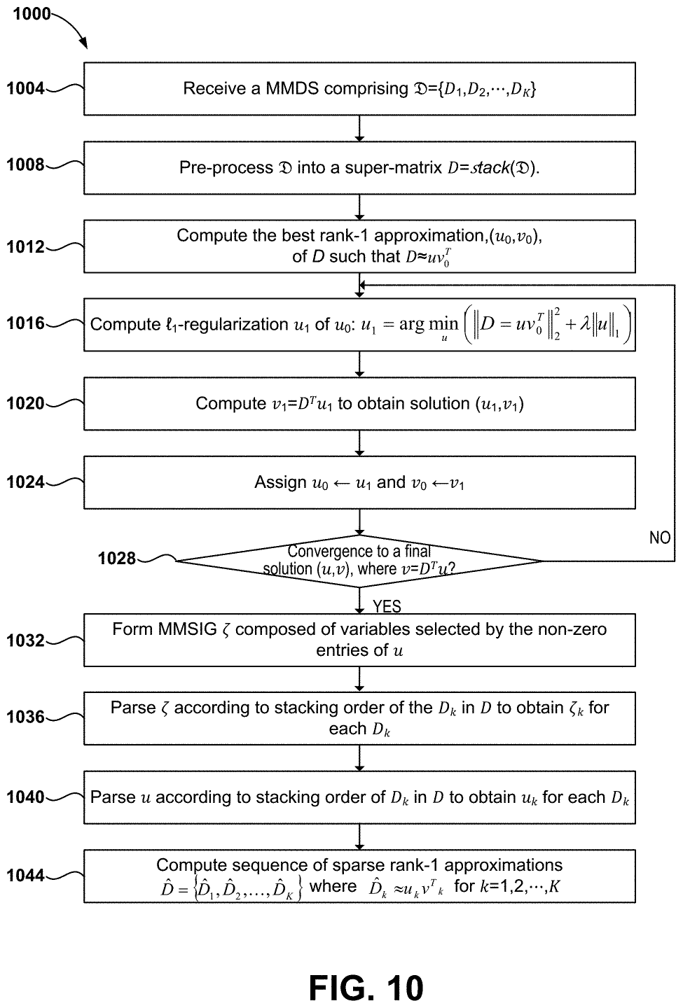

FIG. 10 is an exemplary flowchart outlining the steps of a single iteration of the JAMMIT algorithm for computing joint rank-1 approximations of each Dₖ of a given super-matrix D. At block 1004, the method 1000 receives a MMDS dataset D. The dataset D 1004 can be {D_{1, 2}, . . . , D_{K}}. Let [symbol] ={Dₖ}ₖ₋₁^{K} denote a collection of pₖ×n data matrices Dₖ that represents a MMDS acquired from a common set of n biospecimens, Sₙ{ζ₁, ζ₂, . . . , ζₙ}.

At block 1004, the method 1000 can pre-process super-matrix D=stack( [symbol] ). Let D=stack( [symbol] ) denote the p×n super-matrix for D, where p=Σhd k=1^{m} pₖ. In some embodiments, at least one Dₖ can be assumed to be big, so that the super-matrix D is also big, i.e., p>>n. Each Dₖ can be assumed to have been individually pre-processed as a function of its data type as discussed in the previous section and that D is scaled by its Frobenius norm, that is, if D=[dᵢⱼ] is a p×n matrix, then D←D·/∥D∥_{Frob}, where ∥D∥_{Frob}=(ΣᵢΣⱼ|dᵢⱼ|²)^{1/2} is the Frobenius norm of D; and D·/∥D∥_{Frob}=└dᵢⱼ/∥D∥_{Frob}┘.

At block 1012, the method 1000 computes the best rank-1 approximation, (u_{0,0}) of D such that D≈u₀ v₀^{T}. For λ>0, the JAMMIT generates following rank-1 approximation of D

D≈u(λ)(v(2))^{T}=uv^{T} (1)

by minimizing the error function

E(u,v,λ)=∥d−uv^{T}∥_{Frob}²+λ∥u∥ₗ_{₁} (2)

subject to the constraint

v=D^{T}u (3)

where uv^{T}∈ [symbol] ^{p×n} is the outer product of u∈ [symbol] ^{p} and v∈ [symbol] ⁿ; u is sparse relative to p, i.e., s<<p; v represents a SOI on the columns of D; λ>0 is an l₁ penalty on u; and 5) ∥u∥ₗ_{₁}=Σᵢ₌₁^{p}|uᵢ| is the l₁-norm of u∈ [symbol] ^{p}.

At block 1016, the method 1000 can compute l₁-regularization u₁ of u₀:

$${u}_{1} = \begin{matrix}arg min \\ u\end{matrix} ( {‖ D = {uv}_{0}^{T} ‖}_{2}^{2} + λ {‖ u ‖}_{1} ) .$$

Starting with an initial l₂ approximation (u⁽⁰⁾, V⁽⁰⁾) based on the SVD of D such that D≈u⁽⁰⁾(v⁽⁰⁾)^{T}, JAMMIT can first obtain a l₁-regularized solution vector u¹∈d^{p} defined by

$${u}^{( 1 )} = \begin{matrix}arg min \\ u ∈ {R}^{P}\end{matrix} ( E ( u , {v}^{( 0 )} , λ ) ) , \qquad ( 4 )$$

and then substitutes this solution in (3) to obtain v¹∈ [symbol] ⁿ and the solution (u⁽¹⁾,v⁽¹⁾) that satisfies D=u⁽¹⁾(v⁽¹⁾)^{T}. At block 1020, the method 1000 can compute v₁=D^{T}u₁ to obtain solution (u_{1,1}).

At decision block 1028, the method 1000 can proceed to block 1032 if u₀ and v₀ has converged to a final solution (u, v), where v=D^{T}u. At block 1024, the method 1000 can assign u₀←u₁ and to v₀←v₁. At decision block 1028, the method 1000 can return to block 1016 if u₀ and v₀ has not converged to a final solution (u, v), where v=D^{T}u. Accordingly, in some embodiments, the blocks 1016, 1020, and 1024 can be repeated by alternating between (2) and (3) until the sequence (u⁽ⁱ⁾,v⁽ⁱ⁾) converges to a solution (U,V) based on the error function given in (2) such that

v=D^{T}u=Σₖ₌₁^{m}Dₖ^{T}uₖ. (5)

At block 1032, the method 1000 can form a MMSIG ζ composed of variables selected by the non-zero entries of u. Let ζ(λ)∈ [symbol] ^{s} denote the MMSIG with non-zero entries that select s=s(ζ)>0 rows of D that support the sparse linear model in (5) as a function of λ. Some observations include that: λ=0 implies that (1) is the best rank-1 approximation of D based on the SVD; λ>0 implies that (1) is a l₁-regularized, rank-1 approximation of D such that s=dim(ζ)≤p; and iii) there exists λ^{sup} >0 such that 0≤s≤p if λ∈(0, λ^{suo}). In some embodiments, for simulated and real multi-modal data, λ*∈(0, λ^{sup}) can be found based on an empirical estimate of FDR such that ζ(λ*)=s*<<p.

At block 1036, the method 1000 can parse ζ according to stacking order of the Dₖ in D to obtain ζₖ for each Dₖ. At block 1040, the method 1000 can parse u according to stacking order of Dₖ in D to obtain uₖ for each Dₖ. At block 1044, the method 1000 can compute sequence of sparse rank-1 approximations \hat{D}={\hat{D}₁, \hat{D}₂, . . . , \hat{D}_{K}} where \hat{D}ₖ≈v^{T} for k=1, 2, . . . , K.

Equation (5) suggests that parsing the vector u according to the order in which the Dₖ's were stacked in D results in individual rank-1 approximations

Dₖ≈uₖv^{T} for k=1,2, . . . ,m (6)

such that uₖ∈R^{s}^{ₖ} is unique to each Dₖ and v represents the SOI in (1) that is shared by each Dₖ. Equation (6) implies that the MMSIG ζ*=ζ(λ*)=ζ*(D) can be similarly parsed into type-specific signatures ζₖ*=ζ*(Dₖ) according to the stacking order of the Dₖ's in D that explain v in terms of the kth data type only. In some embodiments, the sparsity of ζ* implies that the type-specific signatures ζₖ* in Dₖ are also sparse if Dₖ is big.

In some embodiments, JAMMIT detects and models the most dominant SOI in D′. This procedure can be iterated until no statistically significant MMSIG are detected and modeled. One non-limiting hypothesis is that the number of iterations is bounded by ₖ^{min} [rank(Dₖ)].

Selecting an l₁ Penalty Based on False Discovery Rate (FDR)

For actual experimental data, empirical FDR can be used to select an ti penalty that results in a MMSIG of desired size and a measure of statistical significance for the MMSIG in D and for each type-specific signature in Dₖ. Briefly, FDR was estimated for a monotone increasing sequence of λ's denoted by

Λ={0=λ₁<λ₂< . . . <λ₁< . . . <λₛᵤₚ<∞} (8)

such that λ=0 results in the MMSIG provided by the SVD and λₛᵤₚ is the smallest λ that results in a MMSIG of length zero. The presence of statistically significant row-correlations between the matrices of D is indicated by a sequence of FDR values)

Θ(Λ)={Θ(λ₁),Θ(λ₂), . . . , Θ(λₛᵤₚ)} (9)

that decreases quickly as a function of increasing λ. In this case aλ*∈Λ can be selected such that: Θ(λ*)∈Θ(Λ) is a local minimum that is smaller than some predetermined threshold; and the resulting signature, ζ*=ζ(λ*), is sparse in D. Conversely, a FDR sequence, Θ(Λ), that fails to decrease fast enough precludes the selection of a λ*∈Λ that is less than a predetermined threshold and implies a lack of row-support from one or more of the Dₖ's for dominant SOI of D. Note that a “joint” FDR sequence, Θ(Λ), can be decomposed into a collection of type-specific FDR sequences Θ(Λ)={Θₖ(Λ)}ₖ₌₁^{K} based on the stacking order of the Dₖ's in D.

In some embodiments, Ok(A) represents the FDR sequence for the kth sub-signature, ζₖ* of ζ* (see the Section below on Computing FDR on a Grid of l₁ Penalties). Again, the presence of a sparse subset of variables in Dₖ that support the common SOI in a statistically significant way is signaled by a rapidly decreasing sequence of FDR values in Θₖ(Λ), while the absence of any row-support Dₖ's is indicated by a slowly decreasing FDR sequence Θₖ(Λ), for k=1, 2, . . . , K. Table 1 provides more detail on how the FDR sequences Θ(Λ) and Λₖ(Λ) were generated. Table 1 shows an exemplary workflow summarizing the generation of a sequence of FDR values based on a monotone increasing sequence of λ's.

TABLE 1
Computing FDR on a Grid of l₁ Penalties.

1. Let Λ = {0=λ₁ < λ₂ < . . . < λ₁ < . . . < λₛᵤₚ < ∞} be a
montonically increasing sequence of
l₁ penalties such that for all λ ϵ Λ, |ζ(λ)=0 only if λ>λₛᵤₚ
where ζ(λ) is the JAMMIT-derived
signature for the l₁ penalty λ.
2. Generate a collection of permuted matrices
Δ={D^{(b)}}_{b=0}^{B} based on the original super-
matrix D where D(0)≡D and each D^{(b)} is obtained
by randomly permuting each row of D for
b=1,2, . . . ,B.
3. For a given λϵ Λ
3a. Apply JAMMIT to matrix D^{(b)} ϵΔ and compute
s^{(b)}(λ)=|ζ^{(b)}λ| for b=0,1,2, . . . ,B where
ζ⁽⁰⁾(λ) is the JAMMIT signature for D and ζ^{(b)}(λ) is
the JAMMIT signature for Dₖ^{(b)} for
k=1,2, . . . ,K.
3b. Compute s^{(med)}(λ)=median ({s^{(b)}(λ)}_{b=1}^{B})
3c. Compute Θ(λ)=π₀(s^{(med)}(λ)/s⁽⁰⁾(λ)) where π₀ is
an estimate of the true proportion of
non-zero loading coefficients of u
(usually set at π₀=0.05).
4. Repeat step (2) for each λ ϵ Λ to define a
joint FDR sequence Θ: Λ→[0,1].
5. Define FDR sequences Θₖ : Λ→[0,1] for each Dₖ
where Θₖ(λ) is equal to Θ(λ) restricted
to the data matrix Dₖ.

The use of FDR to select an appropriate l₁ penalty that balances statistical significance and signature size can provide researchers with considerable flexibility in model selection, but it comes with a high computational cost associated with permutation testing. Future studies should consider alternative methods of selecting an “optimal” l₁ penalty that takes into account user preferences for model parsimony, sensitivity, and specificity without resampling. Finally, this study illustrates the importance of taking a sequential approach to post-analysis data reduction and interpretation of signatures generated by JAMMIT to realize predictive models that generalize robustly to larger populations. For example, the use of pathway analysis to parse a given JAMMIT signature into smaller sub-signatures based on significant upstream regulators was shown to result in low-dimensional signatures that facilitated downstream biological interpretation and validation. Overall, the reduction of big, multi-modal data to low-dimensional signatures of clinical relevance may require a step-wise approach where algorithms such as JAMMIT represent just the first step of the process.

Eigen-Survival Analysis

Let D be a p×n data matrix where p>>n and let t (D) denote the s×n sub-matrix of D composed of rows from D that correspond to variables (i.e., matrix rows) selected by a JAMMIT-derived signature ζ. Alternatively, the columns of ζ(D) can be viewed as “realizations” of the signature ζ in each of the n patients used to formulate D. Let Ω(D) be a 2×n survival data matrix for D where the 1^{st} row contains observed time-to-death for the n patients of D and the 2^{nd} row is a binary indicator of censorship for each patient (0=uncensored, 1=censored). In some embodiments, an eigen-survival model (ESM) can be extracted based on the SVD of ζ(D) to reduce the negative impact of random noise and systematic errors on the prediction of overall survival. The ESM was then used to compute prognostic scores for each patient, and patients with scores in the top and bottom quartiles of scores were identified. The signature ζ(D) was predictive of survival if and only if differences in survival between patients with scores in the top and bottom quartiles were significant in both the KM and Cox regression models with p-value of 0.05 or less. The Section below on Eigen-Survival Modeling of JAMMIT Signatures provides more detail on the workflow used to extract an ESM for a given signature.

Eigen-Survival Modeling of JAMMIT Signatures

Let ζ(D) be a JAMMIT-derived signature in the p×n data matrix D that was decomposed using the SVD to obtain the outer-product representation

ζ(D)=Σᵣ₌₁^{R}sᵣ(uᵣvᵣ^{T}) (10)

where: sᵣ∈R, uᵣ∈R^{s}, and vᵣ∈Rⁿ are the rth singular value, left-singular vector, and right-singular value respectively, for r-1, 2, . . . , R; and R is the rank of ζ(D). Each vᵣ in (10) was tested for association with the survival data in Ω(D) using Kaplan-Meier (KM) analysis with log-rank testing, and Cox regression modeling with age as a covariate. To accomplish this, the n components of vᵣ can be interpreted as “prognostic scores” for each patient and sorted the patients by this score to identify those that fell in the top and bottom quartiles of scores. A given vᵣ was called significant if and only if differences in survival between patients in top and bottom quartiles based on vᵣ are significant in both the KM and Cox regression models with a p-value of 0.05 or less. Given that at least one such vᵣ exists, the following can be defined: B={r|vᵣ is significant in both the KM and Cox models}; U={uᵣ|r∈B}; and V={vᵣ|r∈B}.

Then the eigen-survival model, Θ∈Rⁿ, based on ζ(D) is defined by the linear combination of the vectors in V:

Φ=Σ_{r∈B} sign(r)sᵣvᵣ (11)

where sign(r)=±1 was chosen for each r∈B so that the association of (11) with overall survival was maximized in terms of p-values of in KM and Cox regression models. Note that Φ can also be expressed in terms of singular vectors in U by

Φ^{T}=Σ_{r∈B} sign(r)uᵣ^{T}ζ(d) (12A)

or equivalently.

Φ^{T}=Σ_{r∈B} sign(r)ζ(D)^{T}uᵣ. (12B)

In other words, the jth entry of Φ, i.e., the prognostic score for the jth patient, is the dot product of the jth column of ζ(D), which consists of the measurements of the variables in the signature for that patient, with the test vector Π=Σ_{r∈B} sign(r)uᵣ.

To compute prognostic scores for a set of new patients not included in the original samples, let ζ(n′) be an s×n′ matrix with columns that represent realizations of for n′ patients that were unseen during discovery of ζ. Then following (12B),

Φ′=Σ_{r∈B} sign(r)ζ(n′)^{T}uᵣ (13)

which transforms the columns of ζ(n′) into prognostic scores for these patients based on the eigen-survival model defined by (3B). If KM and Cox regression analysis indicates that Φ′ is significantly associated with overall survival, then the eigen-survival model defined by (3B) can be generalized to a larger population beyond the original n patients that were used to discover ζ.

Ingenuity Pathway Analysis (IPA)

Ingenuity Pathway Analysis (QIAGEN Redwood City, Calif.) was used to rapidly profile a given mRNA signature for enrichment in genes, canonical pathways, biological processes and upstream regulators related to cancer. In particular, IPA's Upstream Regulator Analysis (IPA/URA) feature was used to decompose a given JAMMIT-derived signature into lower-dimensional sub-signatures composed of genes that are targeted by a single upstream regulatory molecule. In this analysis, an upstream regulator can be a chemokine, cytokine, transcription factor, drug, etc. and IPA computes an activation score and intersection p-value for the targeted subset of genes. The activation score measures the consistency between the observed effect of the predicted regulator on the targeted variables in our data and the predicted effect based on current knowledge as encoded in the IPA/KB. The intersection p-value measures the probability of a chance association between the predicted upstream regulator and its downstream targets that reside in a given signature. Note that a predicted upstream regulator does not have to be a member of the signature. Activation scores greater than 2.0 and p-values less than 1.0E-03 are considered significant. Signatures that are “anchored upstream” in this way inherit the function of this regulator and are thus easier to interpret biologically. IPA also generates hypotheses regarding the genes and pathways that may explain the downstream effects of a given signature on biological and disease processes.

Considerations

If the support of a dominant SOI of a big MMDS is supported by small percentage of all measured variables, then l₁ regularization provides an efficient and powerful way to identify this sparse signature. This approach can be encoded in the Joint Analysis of Many Matrices by ITeration (JAMMIT) algorithm that estimates a sparse signal model using an implementation of the LASSO to regularize the best rank-1 matrix approximation based on the SVD of the super-matrix that vertically “stacks” the individual data matrices of a MMDS. By unstacking the super-signature derived by JAMMIT, type-specific models, that characterize important sample attributes of potential clinical relevance in terms of variables of one or more data types, can be obtained. JAMMIT compared favorably with other joint analysis algorithms in the detection of multiple SOI embedded in simulated MMDS over a wide range of SNR scenarios.

The below lists a few technical considerations of the JAMMIT algorithm related to the joint analysis of multiple data types that will require further study. For example, l₁ regularization of the super-matrix that vertically stacks the individual data matrices of a given MMDS results in low-dimensional, multi-modal signatures that are biologically coherent and/or predictive of clinical outcomes. This result assumes that each data matrix is appropriately pre-processed as a function of data type and that the super-matrix based on these pre-processed data matrices is scaled by its Frobenius norm. The sensitivity of JAMMIT-derived signatures to this front-end pre-processing procedure is an open question that should be answered more definitively in future studies.

Another consideration pertains to the accounting for systematic variation in the data that can be assumed to be unique to a given data type. Since JAMMIT attempts to model a dominant source of variation that is shared across multiple data types, the FDR profiles of each data type can be assumed to rapidly decrease in unison as a function of increasing l₁ penalty, if such a signal exists in the super-matrix of the MMDS. In this case, it is unlikely that the resulting signal model will represent systematic variation unique to a single data type. Alternatively, if only a single data type shows a decreasing FDR profile, then it is possible that JAMMIT is modeling a source of systematic variation that is unique to that data type. Subsequent downstream processing of the resulting uni-modal signature using pathway and ontological analysis should be able to resolve some of the ambiguity regarding the biological and/or clinical relevance of such signatures. This feature of JAMMIT to discriminate between systematic and biologically relevant sources of variation based on FDR profile should be characterized more fully in future investigations.

Exemplary JAMMIT Workflow

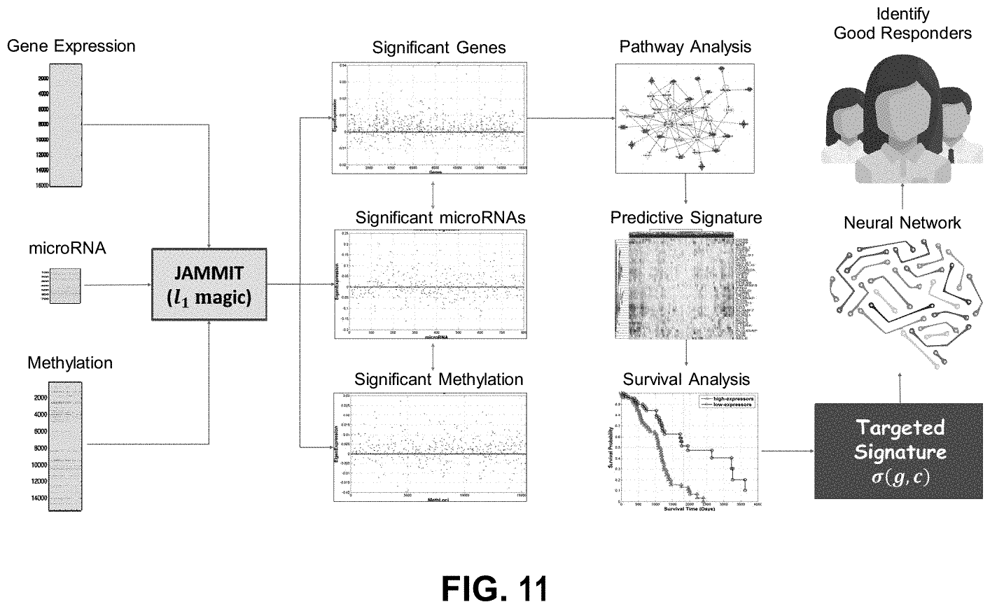

FIG. 11 shows an exemplary workflow for identification of good responders using JAMMIT. Step (1) Heat maps of gene expression, microRNA and DNA methylation data matrices are assembled and pre-processed for input to JAMMIT algorithm. Step (2) JAMMIT analysis with, for example, minus-one cross-validation. Step (3) Scatter plots of sparse eigen-arrays generated by JAMMIT for each data type can be used to determine significant genes, microRNAs, and methylation patterns. Step (4) Pathway analysis of the significant genes can be performed and used for signature determination at Step (5). Step (6) Survival analysis of the predictive signature can be used to determine a targeted signature. Step (7) the targeted signature can be applied to a neural network to identify good responders to, for example, pharmaceuticals.

### Encoding of Targeted Signatures for Immune Checkpoint Blockade (ICB) in Machine Learning Models Based on Wavelet-SVD Features

Overview

Disclosed herein are systems and methods for encoding a given targeted signature for immune checkpoint blockade (ICB) or another biological process in a machine learning model. In some embodiments, the encoding process involves one or more of the following:

1) the physical realization of the signature (e.g., the signature determined using JAMMIT) in a biochemical assay, such as qPCR;
2) the extraction of wavelet-SVD features from the output of the biochemical assay; and
3) the training of a machine learning model (e.g., neural network, support vector machine, random forest, etc.) using wavelet-SVD features.

Also disclosed herein are targeted signatures for immune checkpoint blockade or other biological processes. Such a signature can identify patients who would derive a benefit (e.g., survival benefit or quality of life benefit) from the blockade of a specific checkpoint gene in a specific disease (e.g., a specific cancer). Such a signature can relate gene expression, overall survival, and a targeted checkpoint gene in a specific cancer in a single system.

In some embodiments, a targeted signature that predicts response to checkpoint blockade can satisfy three properties. First, the signature can stratify a discovery cohort of patients in terms of overall survival into a good and poor prognosis groups. Second, the targeted checkpoint gene can be differentially expressed between the good and poor response groups defined by the signature. Finally, the targeted gene can stratify the sub-cohort of patients who belong to the good and poor survival groups defined by the signature. If a signature satisfies all three properties, then it can be used to identify patients who would derive a survival benefit (or another benefit) from blockade of the targeted checkpoint gene. Note that 3 properties of a viable signature can be used to actually identify such signatures using the systems, methods, or processes described herein.

Such a signature can be implemented in a 2-step process described herein. First, a neural network based on the signature can be used to identify candidate patients for blockade of the targeted checkpoint gene. Second, the level of the targeted checkpoint can be measured in a candidate patient identified in first step and a threshold can be applied to the measured value. If the expression of the targeted gene exceeds the applied threshold, then the patient can receive treatment that blocks the target gene.

Disclosed herein are systems and methods for encoding a given targeted signature for immune checkpoint blockade (ICB) or another biological process in a machine learning model. In some embodiments, the method comprises receiving data on a plurality of expression patterns associated with a plurality of realizations ξ_{D}_{_{χ}}_{ⁿ}^{p} of a targeted signature ξ^{p} determined using a plurality of n tissue samples D_{χ}ⁿ obtained from a plurality of n patients with a cancer χ. The plurality of realizations ξ_{D}_{_{χ}}_{ⁿ}^{p} can comprise a realization ξ_{π}^{p} of the targeted signature ξ^{p} for a tissue sample of the plurality of n tissue samples D_{χ}ⁿ of a patient π of the plurality of n patients. The realizations ξ_{π}^{p} can be associated with an observed outcome determination d_{π} of a plurality of outcome determinations Ω. The method can further comprise generating a plurality of exemplars (ωᵢ, dᵢ)∈ [symbol] ^{M}×Ω from the plurality of realizations ξ_{D}_{_{χ}}_{ⁿ}^{p}; and training a machine learning model Φ using the plurality of exemplars. The cancer can comprise an ovarian cancer or a liver cancer. The realizations ξ_{π}^{p} can be determined using a biochemical assay.

In some embodiments, the plurality of realizations ξ_{D}_{_{χ}}_{ⁿ}^{p} comprises a p×n matrix of expression measurements of the p genes of ξ^{p} measured in the plurality of n tissue samples D_{χ}ⁿ. The targeted signature ξ^{p} can comprise a plurality of p genes that depends on a checkpoint gene γ and the cancer χ. The plurality of realizations ξ_{D}_{_{χ}}_{ⁿ}^{p} can comprise a real-valued matrix. The expression patterns of the plurality of realizations ξ_{D}_{_{χ}}_{ⁿ}^{p} can stratify the tissue samples D_{χ}ⁿ into the plurality of outcome determinations Ω. The plurality of outcome determinations Ω can comprise a good outcome group, a poor outcome group, and an uncertain outcome group.

In some embodiments, the checkpoint gene γ is differentially expressed between the good outcome group and the poor outcome group. The checkpoint gene γ can be prognostic in a subset of the plurality of n patients of the plurality of n tissue samples D_{χ}ⁿ restricted to the good outcome group and the poor outcome group. The targeted signature can be prognostic in the plurality of n tissue samples D_{χ}ⁿ.

In some embodiments, the method further comprises one or more of the following: receiving data on a plurality of expression patterns associated with a realization ξ_{π}^{p} of the targeted signature ξ^{p} of a second patient π and data of the second patient π on the cancer γ; determining a predicted outcome determination d_{π} of the plurality of outcome determinations Ω using the machine learning model and the realizations ξ_{D}_{_{χ}}_{ⁿ}^{p} of the targeted signature ξ^{p} of the second patient π; determining the predicted outcome determination d_{π}, comprises a good outcome determination; determining the data of the second patient π on the cancer γ is above a threshold; and determining retreatment of the second patient π with blockade of a checkpoint gene γ is likely to result in a benefit for the second patient π.

In some implementations, determining the predicted outcome determination d_{π} comprises a good outcome determination comprises factoring the plurality of realizations ξ_{D}_{_{χ}}_{ⁿ}^{p} to generate an eigen-survival model (ESM); and projecting the realization ξ_{π}^{p} of the targeted signature ξ^{p} of the second patient π into the eigen-survival model to generate a prognostic score ρ(ξ_{π}^{p}) for the second patient π. The plurality of realizations ξ_{D}_{_{χ}}_{ⁿ}^{p} can be factored using singular value decomposition (SVD) to generate the eigen-survival model (ESM). The prognostic score ρ(ξ_{π}^{p}) can comprise an inner product of the eigen-survival model and the realization ξ_{π}^{p}. Generating the plurality of exemplars (ωᵢ, dᵢ)∈ [symbol] ^{M}×Ω from the plurality of realizations ξ_{D}_{_{χ}}_{ⁿ}^{p} can comprise determining a plurality of wavelet coefficients using the realizations ξ_{π}^{p} and the observed outcome determination d^{π}; filtering the plurality of wavelet coefficients to generate a plurality of L filtered wavelet coefficient; and performing singular value decomposition (SVD) of a data matrix W of the L wavelet coefficients to generate the plurality of exemplars (ωᵢ, dᵢ)∈ [symbol] ^{M}×Ω.

Notations

Let ξ^{p}(γ,χ)=ξ^{p} be a targeted signature composed of p>0 genes that predicts response to blockade of the immune checkpoint gene γ in cancer χ. Let D_{χ}ⁿ be a collection of unique tissue samples obtained from a cohort of n patients with cancer χ annotated by their respective censored survival times. Let ξ^{p}(γ, D_{χ}ⁿ)=ξ_{D}_{_{χ}}_{ⁿ}^{p} denote the realization of ξ^{p} in D₁₀₂ ⁿ. For example, ξ_{D}_{_{χ}}_{ⁿ}^{p} can be a p×n matrix of expression measurements of p genes of ξ^{p} measured in the n tissue samples of D_{χ}ⁿ. Note that in some implementations, ξ^{p} is a list of genes that depends on checkpoint gene γ and cancer of interest χ while ξ_{D}_{_{χ}}_{ⁿ}^{p} is a real-valued matrix. Patients classified as “good responders” by ξ^{p} can be deemed likely to derive a survival benefit from the blockade of γ in χ.

Characterization of Targeted Signatures for ICB

Let ξ_{D}_{_{χ}}_{ⁿ}^{p} be a realization of the targeted signature ξ^{p} in D_{χ}ⁿ. Then ξ_{D}_{_{χ}}_{ⁿ}^{p} can satisfy the following properties:

1) the expression patterns of ξ_{D}_{_{χ}}_{ⁿ}^{p} stratifies the n patients of D_{χ}ⁿ into good, poor and uncertain response groups with significantly different curves (e.g., Kaplan-Meier curves) per the ranking test (e.g., the log rank test). For example, ξ^{p} is prognostic in D_{χ}ⁿ.
2) γ is differentially expressed between the good and poor response groups defined in (1).
3) γ is prognostic in the subset of patients of D_{χ}ⁿ restricted to the good and poor response groups defined in (1).
These three properties characterize the ability of the signature ξ^{p} to identify patients in D_{χ}ⁿ who would derive a survival benefit from the blockade of γ in χ. This result also provides a means to determine whether a given gene signature obtained by JAMMIT, or some other data reduction method or technique, is in fact a targeted signature for ICB.

Signature ξ^{p} as a Biochemical Assay

In some embodiments, the signature ξ^{p} can be realized as a p-dimensional, real-valued vector ξ_{π}^{p}=ξ^{p}(γ, π∈D_{χ}ⁿ)∈ [symbol] ^{p} for a tumor obtained from a patient π by a biochemical assay that measures mRNA levels of multiple genes in a given tissue sample, such as quantitative polymerase chain reaction (qPCR). A realization ξ_{π}^{p} of ξ^{p} for the patient π can be presented as input to a machine learning model, Φ, such that Φ(ξ_{π}^{p})=d_{π}∈Ω where Ω is a finite set of targeted outcomes. The finite set Ω of targeted outcomes can be defined by

$$Ω = \{ \begin{matrix}good responder \\ poor responder \\ uncertain responder\end{matrix} .$$

The assay can act as a measurement platform that transforms a given tissue sample into a p-dimensional vector of real numbers suitable for presentation to the input layer of a machine learning model.

Clinical Application of P

In some implementations, application (e.g., clinical application) of ξ^{p} can include two steps. Step 1: For a new patient, π, the mRNA levels of ξ^{p} and γ are measured in the patient's tumor. The resulting p-dimensional vector ξ_{π}^{p}∈ [symbol] ^{p} is then “mapped” to a response outcome in Ω where only patients that are assigned a “good response” outcome are candidates for ICB.

Step 2: For a patient who is a candidate for ICB, a threshold is applied to the expression level of γ in the tumor. If the threshold is exceeded, then the patient receives ICB treatment targeting γ; otherwise not. Bioinformatics analysis of data from The Cancer Genome Atlas (TCGA) for ovarian cancer, liver cancer, or another cancer can suggest that ICB response can be significantly increased by restricting ICB only to patients identified using the 2-step procedure described above.

Machine Learning Model

One aspect in the clinical application of ξ^{p} includes assigning to ξ_{π}^{p} a response outcome in Ω. This decision process can be mathematically represented as a mapping Φ: [symbol] ^{P}→Ω where [symbol] ^{p} is p-dimensional Euclidean space and Φ(ξ_{π}^{p})=dₖ represents a prediction of response based on ξ_{π}^{p}. In some embodiments, the mapping Φ can be implemented as a machine learning model (e.g., neural network, support vector machine, random forest, etc.) trained on K exemplars denoted by (ξᵢ^{p},dᵢ)∈ [symbol] ^{P}×Ω for i=1, 2, . . . , n, where ξᵢ^{p} is a realization of V in a tissue sample of the ith patient of D_{χ}ⁿ and dᵢ∈Ω is the response class assigned to the ith patient based on an eigen-survival analysis of a cohort of patients with censored survival data that is used to derive ξ^{p}.

Through the process of training, the weights of the machine learning model can be incrementally or iteratively adapted such that the output of the network, given a particular input data from a training set comprising the K exemplars, comes to match (e.g., as closely as possible) the target output corresponding to that particular input data. In some embodiments, the machine learning model comprises a classification model. The classification model can comprise a supervised classification model, a semi-supervised classification model, an unsupervised classification model, or a combination thereof. The machine learning model can comprise a neural network, a linear regression model, a logistic regression model, a decision tree, a support vector machine, a Nave Bayes network, a k-nearest neighbors (KNN) model, a k-means model, a random forest model, or any combination thereof.

Eigen-Survival Analysis Based on V

Let ξ^{p}(γ,D_{χ}ⁿ)=ξ_{D}_{_{χ}}_{ⁿ}^{p} be a p×n realization of ξ^{p} in D_{χ}ⁿ. Let Σ be a 2×n survival data matrix aligned with columns of D_{χ}ⁿ where the 1st row contains observed time-to-death for the n patients of D and the 2^{nd} row is a binary indicator of censorship for each patient (0=uncensored, 1=censored). ξ_{D}_{_{χ}}_{ⁿ}^{p} can be factored based on the Singular Value Decomposition (SVD) to define an eigen-survival model (ESM) based the top right-singular vector of the factorization to reduce the impact of random noise and systematic errors on the prediction of overall survival. The signatures ξ_{π}^{p} of a given patient, π, in D_{χ}ⁿ is then projected onto the ESM using ρ: [symbol] ^{p}→ [symbol] defined by ρ(ξ_{π}^{p})=inner product of the ESM and ξ_{π}^{p} to arrive at a prognostic score for each patient, and patients with scores in the top and bottom quartiles of scores can be identified. In some embodiments, the signature ξ_{D}_{_{χ}}_{ⁿ}^{p} can be prognostic if and/or only if differences in survival between patients with scores in the top and bottom quartiles are significant in both KM and Cox regression models with p-values of 0.20, 0.10, 0.05, 0.01, or less.

SVD Compression in Wavelet Space for the Enhanced Prediction of Response to ICB Using Neural Networks

Let (ξᵢ^{p},dᵢ)␣ [symbol] ^{P}×Ω for i=1, 2, . . . , n be a collection of exemplar, where ξᵢ^{p} is a realization of ξ^{p} in the tissue sample for the ith patient of D_{χ}ⁿ and dᵢ∈Ω is the response outcome associated with ξᵢ^{p}. The p×n data matrix E can be formed with columns represented by ξᵢ^{p} for i=1, 2, . . . , n. The wavelet transform of a given column of E can be computed. The resulting coefficients can be filtered to obtain L wavelet coefficients that represent the signal sub-space of the column. Doing this for each column results in a L×n data matrix W of wavelet coefficients. The SVD of W can be computed and the M×n projection matrix P can be formed based on the top M eigenvectors of the SVD of W. Each column of W can be projected onto the projection matrix P to obtain an M-dimensional vector which forms the ith column of the M×K training data matrix T. The “target” values dᵢ can be aligned to their respective columns of T to obtain exemplars (ωᵢ, dᵢ)∈ [symbol] ^{M}×Ω for i=1, 2, . . . , n where ωᵢ is the M-dimensional vector of wavelet-SVD features for the ith patient of D_{χ}ⁿ and dᵢ is the observed outcome associated with ωᵢ.

The exemplars (ωᵢ,dᵢ) can be used to train a neural network (NN), such as a neural network classifier (NNC), with M input nodes and a number of output nodes (e.g., three output nodes), where ωᵢ represents the M-dimensional training data for the ith patient and each output node represents probability of membership in one of the response groups (e.g., the good, poor or uncertain response groups) of the eigen-survival model of D_{χ}ⁿ, respectively. Genetic programming can be used to optimize NNC architecture and internal parameters or weights of the NN model to enhance generalizability to data unseen during training.

For a new patient, the following can be performed: profile the patient's tissue sample to generate ξ_{new}^{p}; compute the wavelet transform of ξ_{new}^{p}; filter down to L wavelet coefficients based on scale; project the L coefficients onto the projection matrix of W to obtain a M-dimensional feature vector ω_{new}; present ω_{Onew} to the input layer of the trained NNC; and assign an outcome d_{new}∈Ω to the input vector ω_{new} that corresponds to the node with the highest probability of membership.

### Cancers

Non-limiting examples of cancer include acute lymphoblastic leukemia, adult; acute myeloid leukemia, adult; adrenocortical carcinoma; aids-related lymphoma; anal cancer; bile duct cancer; bladder cancer; brain tumors, adult; breast cancer; breast cancer and pregnancy; breast cancer, male; carcinoid tumors, gastrointestinal; carcinoma of unknown primary; cervical cancer; chronic lymphocytic leukemia; chronic myelogenous leukemia; chronic myeloproliferative neoplasms; cns lymphoma, primary; colon cancer; endometrial cancer; esophageal cancer; extragonadal germ cell tumors; fallopian tube cancer; gallbladder cancer; gastric cancer; gastrointestinal carcinoid tumors; gastrointestinal stromal tumors; germ cell tumors, extragonadal; germ cell tumors, ovarian; gestational trophoblastic disease; hairy cell leukemia; hepatocellular (liver) cancer, adult primary; histiocytosis, langerhans cell; hodgkin lymphoma, adult; hypopharyngeal cancer; intraocular (eye) melanoma; islet cell tumors, pancreatic neuroendocrine tumors; kaposi sarcoma; kidney (renal cell) cancer; kidney (renal pelvis and ureter, transitional cell) cancer; langerhans cell histiocytosis; laryngeal cancer; leukemia, adult acute lymphoblastic; leukemia, adult acute myeloid; leukemia, chronic lymphocytic; leukemia, chronic myelogenous; leukemia, hairy cell; lip and oral cavity cancer; liver cancer, adult primary; lung cancer, non-small cell; lung cancer, small cell; lymphoma, adult Hodgkin; lymphoma, adult non-hodgkin; lymphoma, aids-related; lymphoma, primary cns; malignant mesothelioma; melanoma; melanoma, intraocular (eye); merkel cell carcinoma; metastatic squamous neck cancer with occult primary; multiple myeloma and other plasma cell neoplasms; mycosis fungoides and the sezary syndrome; myelodysplastic syndromes; myelodysplastic/myeloproliferative neoplasms; myeloproliferative neoplasms, chronic; paranasal sinus and nasal cavity cancer; nasopharyngeal cancer; neck cancer with occult primary, metastatic squamous; non-hodgkin lymphoma, adult; non-small cell lung cancer; oral cavity cancer, lip oropharyngeal cancer; ovarian epithelial cancer; ovarian germ cell tumors; ovarian low malignant potential tumors; pancreatic cancer; pancreatic neuroendocrine tumors (islet cell tumors); pheochromocytoma and paraganglioma; paranasal sinus and nasal cavity cancer; parathyroid cancer; penile cancer; pheochromocytoma and paraganglioma; pituitary tumors; plasma cell neoplasms, multiple myeloma and other; breast cancer and pregnancy; primary peritoneal cancer; prostate cancer; rectal cancer; renal cell cancer; transitional cell renal pelvis and ureter; salivary gland cancer; sarcoma, Kaposi; sarcoma, soft tissue, adult; sarcoma, uterine; mycosis fungoides and the sezary syndrome; skin cancer, melanoma; skin cancer, nonmelanoma; small cell lung cancer; small intestine cancer; stomach (gastric) cancer; testicular cancer; thymoma and thymic carcinoma; thyroid cancer; transitional cell cancer of the renal pelvis and ureter; trophoblastic disease, gestational; carcinoma of unknown primary; urethral cancer; uterine cancer, endometrial; uterine sarcoma; vaginal cancer; and vulvar cancer.

In some embodiments, non-limiting examples of cancer include, but are not limited to, hematologic and solid tumor types such as acoustic neuroma, acute leukemia, acute lymphoblastic leukemia, acute myelogenous leukemia (monocytic, myeloblastic, adenocarcinoma, angiosarcoma, astrocytoma, myelomonocytic and promyelocytic), acute t-cell leukemia, basal cell carcinoma, bile duct carcinoma, bladder cancer, brain cancer, breast cancer (including estrogen-receptor positive breast cancer), bronchogenic carcinoma, Burkitt's lymphoma, cervical cancer, chondrosarcoma, chordoma, choriocarcinoma, chronic leukemia, chronic lymphocytic leukemia, chronic myelocytic (granulocytic) leukemia, chronic myelogenous leukemia, colon cancer, colorectal cancer, craniopharyngioma, cystadenocarcinoma, dysproliferative changes (dysplasias and metaplasias), embryonal carcinoma, endometrial cancer, endotheliosarcoma, ependymoma, epithelial carcinoma, erythroleukemia, esophageal cancer, estrogen-receptor positive breast cancer, essential thrombocythemia, Ewing's tumor, fibrosarcoma, gastric carcinoma, germ cell testicular cancer, gestational trophoblastic disease, glioblastoma, head and neck cancer, heavy chain disease, hemangioblastoma, hepatoma, hepatocellular cancer, hormone insensitive prostate cancer, leiomyosarcoma, liposarcoma, lung cancer (including small cell lung cancer and non-small cell lung cancer), lymphangioendothelio-sarcoma, lymphangiosarcoma, lymphoblastic leukemia, lymphoma (lymphoma, including diffuse large B-cell lymphoma, follicular lymphoma, Hodgkin's lymphoma and non-Hodgkin's lymphoma), malignancies and hyperproliferative disorders of the bladder, breast, colon, lung, ovaries, pancreas, prostate, skin and uterus, lymphoid malignancies of T-cell or B-cell origin, leukemia, medullary carcinoma, medulloblastoma, melanoma, meningioma, mesothelioma, multiple myeloma, myelogenous leukemia, myeloma, myxosarcoma, neuroblastoma, oligodendroglioma, oral cancer, osteogenic sarcoma, ovarian cancer, pancreatic cancer, papillary adenocarcinomas, papillary carcinoma, peripheral T-cell lymphoma, pinealoma, polycythemia vera, prostate cancer (including hormone-insensitive (refractory) prostate cancer), rectal cancer, renal cell carcinoma, retinoblastoma, rhabdomyosarcoma, sarcoma, sebaceous gland carcinoma, seminoma, skin cancer, small cell lung carcinoma, solid tumors (carcinomas and sarcomas), stomach cancer, squamous cell carcinoma, synovioma, sweat gland carcinoma, testicular cancer (including germ cell testicular cancer), thyroid cancer, Waldenstrom's macroglobulinemia, testicular tumors, uterine cancer, Wilms' tumor and the like.

Non-limiting examples of the cancer include acute lymphoblastic leukemia, childhood; acute myeloid leukemia/other myeloid malignancies, childhood; adrenocortical carcinoma, childhood; astrocytomas, childhood; atypical teratoid/rhabdoid tumor, childhood central nervous system; basal cell carcinoma, childhood; bladder cancer, childhood; bone, malignant fibrous histiocytoma of and osteosarcoma; brain and spinal cord tumors overview, childhood; brain stem glioma, childhood; (brain tumor), childhood astrocytomas; (brain tumor), childhood central nervous system atypical teratoid/rhabdoid tumor; (brain tumor), childhood central nervous system embryonal tumors; (brain tumor), childhood central nervous system germ cell tumors; (brain tumor), childhood craniopharyngioma; (brain tumor), childhood ependymoma; breast cancer, childhood; bronchial tumors, childhood; carcinoid tumors, childhood; carcinoma of unknown primary, childhood; cardiac (heart) tumors, childhood; central nervous system atypical teratoid/rhabdoid tumor, childhood; central nervous system embryonal tumors, childhood; central nervous system germ cell tumors, childhood; cervical cancer, childhood; chordoma, childhood; colorectal cancer, childhood; craniopharyngioma, childhood; effects, treatment for childhood cancer, late; embryonal tumors, central nervous system, childhood; ependymoma, childhood; esophageal tumors, childhood; esthesioneuroblastoma, childhood; ewing sarcoma; extracranial germ cell tumors, childhood; gastric (stomach) cancer, childhood; gastrointestinal stromal tumors, childhood; germ cell tumors, childhood central nervous system; germ cell tumors, childhood extracranial; glioma, childhood brain stem; head and neck cancer, childhood; heart tumors, childhood; hematopoietic cell transplantation, childhood; histiocytoma of bone, malignant fibrous and osteosarcoma; histiocytosis, langerhans cell; hodgkin lymphoma, childhood; kidney tumors of childhood, wilms tumor and other; langerhans cell histiocytosis; laryngeal cancer, childhood; late effects of treatment for childhood cancer; leukemia, childhood acute lymphoblastic; leukemia, childhood acute myeloid/other childhood myeloid malignancies; liver cancer, childhood; lung cancer, childhood; lymphoma, childhood Hodgkin; lymphoma, childhood non-Hodgkin; malignant fibrous histiocytoma of bone and osteosarcoma; melanoma, childhood; mesothelioma, childhood; midline tract carcinoma, childhood; multiple endocrine neoplasia, childhood; myeloid leukemia, childhood acute/other childhood myeloid malignancies; nasopharyngeal cancer, childhood; neuroblastoma, childhood; non-hodgkin lymphoma, childhood; oral cancer, childhood; osteosarcoma and malignant fibrous histiocytoma of bone; ovarian cancer, childhood; pancreatic cancer, childhood; papillomatosis, childhood; paraganglioma, childhood; pediatric supportive care; pheochromocytoma, childhood; pleuropulmonary blastoma, childhood; retinoblastoma; rhabdomyosarcoma, childhood; salivary gland cancer, childhood; sarcoma, childhood soft tissue; (sarcoma), ewing sarcoma; (sarcoma), osteosarcoma and malignant fibrous histiocytoma of bone; (sarcoma), childhood rhabdomyosarcoma; (sarcoma) childhood vascular tumors; skin cancer, childhood; spinal cord tumors overview, childhood brain and; squamous cell carcinoma (skin cancer), childhood; stomach (gastric) cancer, childhood; supportive care, pediatric; testicular cancer, childhood; thymoma and thymic carcinoma, childhood; thyroid tumors, childhood; transplantation, childhood hematopoietic; childhood carcinoma of unknown primary; unusual cancers of childhood; vaginal cancer, childhood; vascular tumors, childhood; and wilms tumor and other childhood kidney tumors.

Non-limiting examples of cancer include embryonal rhabdomyosarcoma, pediatric acute lymphoblastic leukemia, pediatric acute myelogenous leukemia, pediatric alveolar rhabdomyosarcoma, pediatric anaplastic ependymoma, pediatric anaplastic large cell lymphoma, pediatric anaplastic medulloblastoma, pediatric atypical teratoid/rhabdoid tumor of the central nervous system, pediatric biphenotypic acute leukemia, pediatric Burkitts lymphoma, pediatric cancers of Ewing's family of tumors such as primitive neuroectodermal rumors, pediatric diffuse anaplastic Wilm's tumor, pediatric favorable histology Wilm's tumor, pediatric glioblastoma, pediatric medulloblastoma, pediatric neuroblastoma, pediatric neuroblastoma-derived myelocytomatosis, pediatric pre-B-cell cancers (such as leukemia), pediatric osteosarcoma, pediatric rhabdoid kidney tumor, pediatric rhabdomyosarcoma, and pediatric T-cell cancers such as lymphoma and skin cancer.

### Computing Device

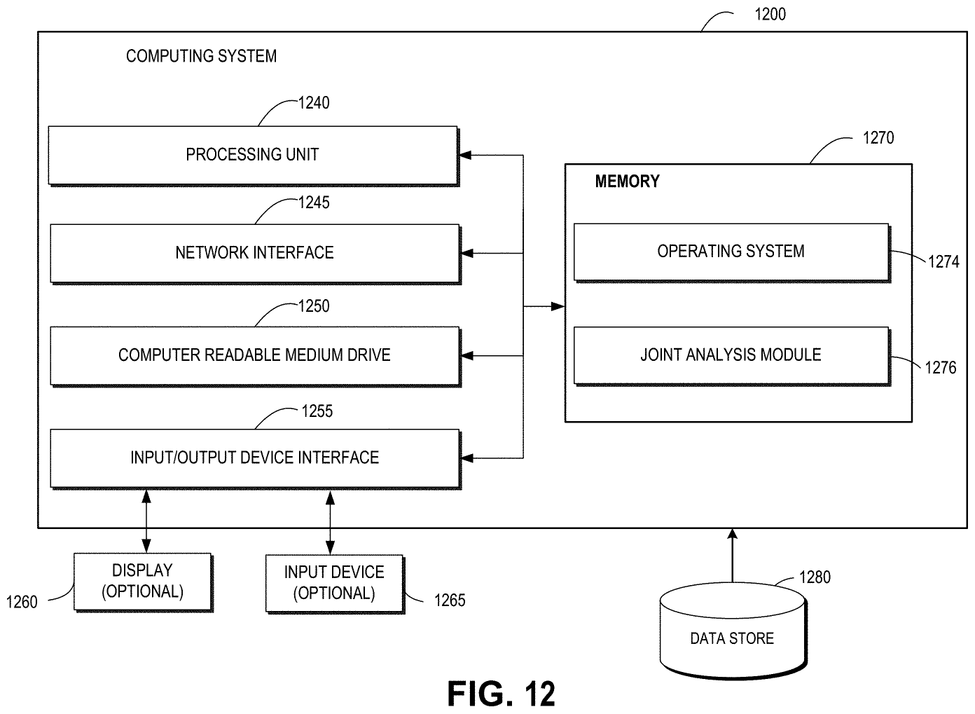

FIG. 12 depicts a general architecture of an example computing device 1200 configured to learn a demographic model and generate a prediction result using the model. The general architecture of the computing device 1200 depicted in FIG. 12 includes an arrangement of computer hardware and software components. The computing device 1200 may include many more (or fewer) elements than those shown in FIG. 12. It is not necessary, however, that all of these generally conventional elements be shown in order to provide an enabling disclosure. As illustrated, the computing device 1200 includes a processing unit 1240, a network interface 1245, a computer readable medium drive 1250, an input/output device interface 1255, a display 1260, and an input device 1265, all of which may communicate with one another by way of a communication bus. The network interface 1245 may provide connectivity to one or more networks or computing systems. The processing unit 1240 may thus receive information and instructions from other computing systems or services via a network. The processing unit 1240 may also communicate to and from memory 1270 and further provide output information for an optional display 1260 via the input/output device interface 1255. The input/output device interface 1255 may also accept input from the optional input device 1265, such as a keyboard, mouse, digital pen, microphone, touch screen, gesture recognition system, voice recognition system, gamepad, accelerometer, gyroscope, or other input device.

The memory 1270 may contain computer program instructions (grouped as modules or components in some embodiments) that the processing unit 1240 executes in order to implement one or more embodiments. The memory 1270 generally includes RAM, ROM and/or other persistent, auxiliary or non-transitory computer-readable media. The memory 1270 may store an operating system 1272 that provides computer program instructions for use by the processing unit 1240 in the general administration and operation of the computing device 1200. The memory 1270 may further include computer program instructions and other information for implementing aspects of the present disclosure. For example, in one embodiment, the memory 1270 includes a joint analysis module 1274 that performs joint analysis of multiple high-dimensional data types using sparse matrix. In addition, memory 1270 may include or communicate with data store 1290 and/or one or more other data stores that store data for analysis or analysis results.

### EXAMPLES

Some aspects of the embodiments discussed herein are disclosed in further detail in the following examples, which are not in any way intended to limit the scope of the present disclosure.

### Example 1

### JAMMIT Performance on Simulated Data

This example evaluates the effectiveness of JAMMIT to detect multiple signals in simulated data sets. This example also compares the effectiveness of JAMMIT to other algorithms such the Joint and Individual Variation Explained (JIVE) and Partial Least Squares (PLS).

JIVE is a generalization of principal components analysis (PCA) to multiple data matrices. Like JAMMIT, PLS enables the supervised analysis of one matrix by another matrix and is also used for the analysis high-dimensional data sets. All three algorithms were applied to the same collection of 1000 simulated MDS's (see Methods section, Simulated Data) and tasked to detect two sparsely supported signals (denoted by SS1 and SS2) over a wide-range of randomly selected SNR scenarios. Recall SS1 is based on a noisy step signal supported by a sparse subset of rows in both simulated data matrices that clusters the 50 simulated samples into two well-defined groups. SS2 is a random signal that is sparsely supported by rows in both matrices that are selected independently from the rows that support the SS1 step signal. Note that SS1 represents a signal for differential expression between two groups of patients while SS2 represents a signal that represents an unmeasured and/or unknown biological attribute of the samples of the simulation.

### Simulated Data

The detection performance of JAMMIT and other joint analysis algorithms were evaluated on 1000 simulated MMDS using Receiver Operating Characteristic (ROC) ANALYSIS. Simulated MMDS, D⁽ⁿ⁾={Dₖ⁽ⁿ⁾}ₖ₌₁²={(Σₖ⁽ⁿ⁾+Nₖ⁽ⁿ⁾)}ₖ₌₁² for n=1, 2, . . . , 1000 were generated where p₁ and p₂ were randomly selected from P={1000,2000,10000}. Σₖ⁽ⁿ⁾ and Nₖ⁽ⁿ⁾ represent simulated signal- and noise-only data matrices, respectively, of dimensions pₖ⁽ⁿ⁾ ×50 for k=1, 2 and n-1, 2, . . . , 1000. For each n, the super-matrix D⁽ⁿ⁾=stack(D⁽ⁿ⁾)=stack(D₁⁽ⁿ⁾,D₂⁽ⁿ⁾)=Σ⁽ⁿ⁾+N⁽ⁿ⁾, was assembled where: 1) p⁽ⁿ⁾=p₁⁽ⁿ⁾=p₂⁽ⁿ⁾; 2) (Σ₁⁽ⁿ⁾,Σ₂⁽ⁿ⁾); and 3) N⁽ⁿ⁾=stack (N₁⁽ⁿ⁾,N₂⁽ⁿ⁾). The support of Σₖ⁽ⁿ⁾ in Dₖ⁽ⁿ⁾ denoted by Supp(Dₖ⁽ⁿ⁾), corresponds to the non-zero components of Iₙ=stack(Iₖⁿ(step),Iₖ⁽ⁿ⁾(rand)) that identify the rows of Dₖ⁽ⁿ⁾ that contain signals SS1 or SS2 defined on the 50 columns of each super-matrix D⁽ⁿ⁾. The signal-to-noise ratio (SNR) or D⁽ⁿ⁾ in decibels is given by

$$S N R ( {D}^{( n )} ) = 10 \times {log}_{10} ( \frac{var ( {\hat{\sum }}^{( n )} )}{var ( {\hat{N}}^{( n )} )} )$$

where \hat{Σ}⁽ⁿ⁾,\hat{N}⁽ⁿ⁾∈R^{50p} represent vectorized versions of Σ⁽ⁿ⁾ and N⁽ⁿ⁾, respectively. The goal of each simulation is to detect Supp(D₍ₙ₎) such that the true positive rate is maximized for a given false positive rate over a wide range of SNR scenarios. The Section below on Generation of Simulated MMDS provides more detail on the generation of simulated signal-only Σₖ⁽ⁿ⁾ and noise-only Nₖ⁽ⁿ⁾ data matrices for n=1, 2, . . . , 1000.

### Generation of Simulated MMDS

Each Dₖ was additively modeled by Dₖ.=Eₖ.+Nₖ for k=1, 2 where Σₖ and Nₖ are pₖ×50 signal-only and noise-only data matrices, respectively, that were simulated as follows:

1. Generate the pₖ×50 matrix, Σₖ, composed of zeros.

2. Let Iₖ(step) and Iₖ(rand) be indicator functions that randomly select 2 subsets of row-indicates from Σₖ, denoted by Suppₖ(step) and Suppₖ(rand), such that Suppₖ(step)∩Suppₖ(rand)=ø; and b) |Suppₖ(step)|+|Suppₖ(rand)|<<pᵢ.

3. For each i∈Suppₖ(step) replace the ith row of Σₖ with /stepᵢ^{T}∈R⁵⁰ defined by

$${step}_{i} = \{ \begin{matrix}{α}_{i} , j = 1 , 2 , … , 25 \\ + {α}_{i} , j = 26 , … , 50\end{matrix}$$

where αᵢ∈Λₛₜₑₚ for some predetermined interval of positive real numbers Λₛₜₑₚ. The collection of rows Σₖ that are replaced by stepᵢ define a multiplexed, step-signal, msetpᵢ∈R^{|SUPP}^{ₖ}^{(step)|×50}, that is supported by the rows in Suppₖ (step) over the 50 columns of Σₖ. Note the variance of the rows of the multiplexed signal, mstepᵢ, varies with the amplitude of stepᵢ.

4. For each i∈Suppₖ(rand) replace the ith row of Σₖ with the random vector randᵢ^{T}∈R⁵⁰ with components sampled from a zero-mean, Gaussian distribution with variance σᵢ². The collection of rows of Σₖ that contain randᵢ define a multiplexed, random-signal, mrandᵢ∈R^{|Supp}^{ₖ}^{(rand)×50|, that is supported by the rows in Supp, (rand) over the} 50 columns of Σₖ. Note the variance of the rows of the multiplexed signal, mrandᵢ, varies with σᵢ².

5. For k-1, 2, define Nₖ as the pₖ×50 random matrix with entries from a zero-mean Gaussian distribution of variance σₖ.

6. For k=1, 2, define Dₖ=Σₖ+Nₖ.

Steps 1-6 were repeated 1000 times where each repetition resulted in a MMDS, D^{(η)}={Dₖ^{(η)}}ₖ₌₁²={(Σₖ^{(η)+N}ₖ^{(η)})}ₖ₌₁², for η=1, 2, . . . , 1000.

### Receiver Operating Characteristic (ROC) Curves Parameterized by the l₁ Penalty Parameter

JAMMIT analysis of a simulated stacked matrix requires the specification of an l₁ penalty parameter λ>0 in equation (2), which results in a signature ζ(λ) such that s=dim(ζ(λ)). The regularized minimization of (2) can be equivalent to the un-regulated minimization of E(u,v)=∥S−uv^{T}∥₂² constrained by ∥u(λ)∥₁≤1/λ, where the l₁ penalty λ result in lower-dimensional signatures. Hence, for a given simulated MMDS and λ>0, the sensitivity and specificity of JAMMIT to detect a given subset of rows of D that support a simulated SOI in the row-space of D can be computed. Consider the monotonically increasing sequence of λₖ's denoted by A and defined in (8). Then sensitivity and specificity for each λ to generate a ROC curve can be computed. Area under the ROC curve (AUROC) was used to quantify the ability of JAMMIT to detect the true support for a simulated signal embedded in a simulated super-matrix D. The detection performance of JAMMIT and any other joint analysis algorithm can be compared by computing the difference between the AUROC values for JAMMIT and the other algorithm (ΔAUROC). A positive ΔAUROC value implies JAMMIT outperformed the other algorithms, otherwise, the other algorithm outperformed JAMMIT.

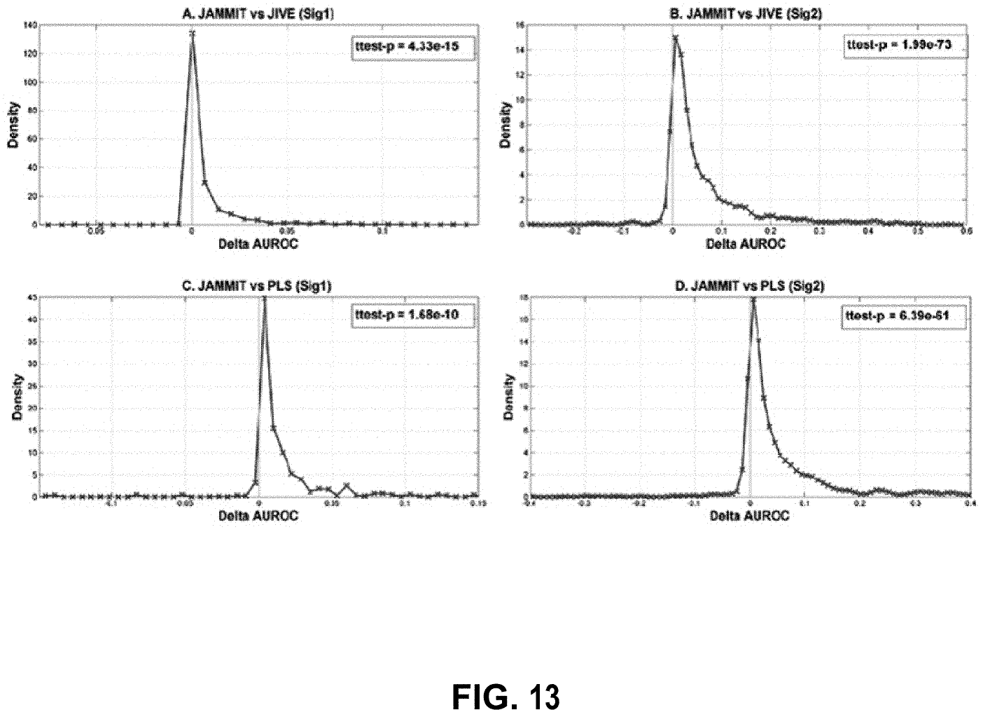

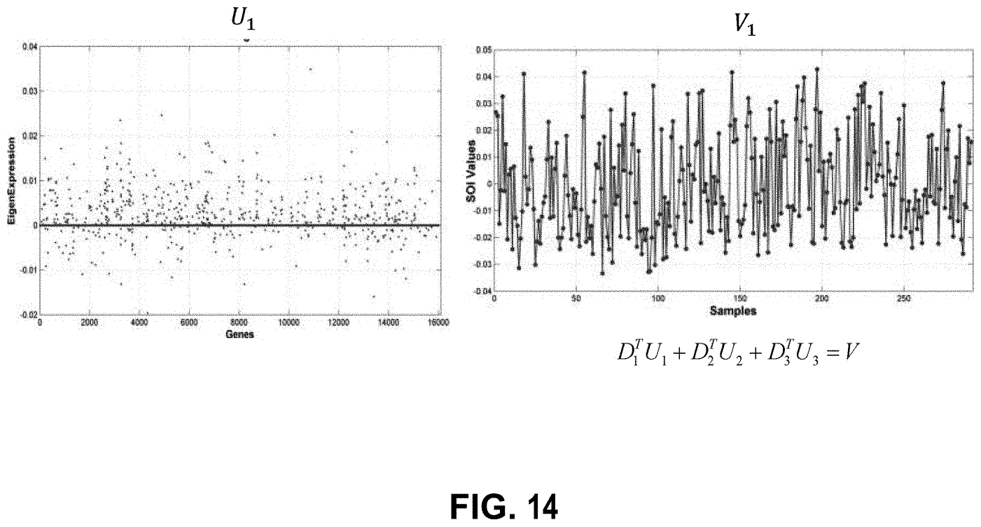

The goal of each simulation is to detect the sparse support of SS1 and SS2 in each simulated data matrix. FIG. 13 shows distributions of Δ AUROC values that compares the ability of JAMMIT to detect the sparse support of SS1 and SS2 versus that of JIVE and PLS over 1000 data simulations. The 1st row of panels show the distributions of ΔAUROC values equal to the AUROC for JAMMIT minus the AUROC for JIVE in the detection of two distinct signals in 1000 simulated MMDS as described in the Methods section of this paper. Similarly, the 2nd row of panels show ΔAUROC distributions for JAMMIT versus PLS to detect the two simulated signals in the same set of simulated MMDS used to evaluate JAMMIT versus JIVE. Each ΔAUROC distribution was based on a normal kernel smoothing function evaluated at 100 equally spaces points using MATLAB's ksdensity function. Note for each distribution, the area under the distribution curve is equal to one and most of this area (i.e. probability measure) is concentrated on the positive x-axis to the right of the vertical green line positioned at x=0. This result indicates that on average JAMMIT outperformed both JIVE and PLS in detecting the two simulated signals over a wide range of SNR scenarios.

For example, the first row of plots show that the distribution of A AUROC values for SS1 and SS2 is concentrated on the positive real axis. This means that the AUROC values for JAMMIT exceeded that of JIVE more frequently than not for SS1 and SS2, with p-values of 4.33.E-15 and 1.99E-73, respectively. Similarly, the second row of plots shows that the area under the A AUROC distributions for both SS1 and SS2 is concentrated on the positive real numbers indicating that JAMMIT outperformed PLS significantly more often than not over 1000 data simulations with p-values of 1.68E-10 and 6.39E-61, respectively. Hence, relative to JIVE and PLS, that JAMMIT compares favorably in terms of ability to detect the sparse support of a step and random signal in multiple, high-dimensional data sets.

Altogether, these data demonstrate that the JAMMIT method outperforms other joint analysis algorithms on simulated MMDS.

### Example 2

### JAMMIT Analysis of Ovarian Cancer Data from TCGA

This example describes application of JAMMIT to MMDS for ovarian cancer.

MMDS for ovarian cancer was downloaded from TCGA resulted in novel, low-dimensional signatures that linked overall survival to immune-cell morphology and macrophage polarization in the tumor microenvironment. Genome-wide mRNA, microRNA and DNA methylation data obtained from 291 tumor samples from patients with clinical stage 3 serous ovarian cancer were downloaded from TCGA (http://cancergenome.nih.gov/). This data download resulted in three high-dimensional data matrices of dimensions 16020×291 (mRNA), 799×291 (microRNA) and 15418×291 (DNA methylation) that were combined to form an ovarian MMDS denoted by D_{OVCA} Meta-data for each patient, which included censored survival time, age, tumor stage and treatment data, were also downloaded from TCGA and aligned with the super-matrix of D_{OVCA}. Subsequent to the assembly of D_{OVCA}, whole-genome mRNA data for an additional 99 tumor samples were downloaded from TCGA along with the appropriate clinical metadata. These data were organized to form a mRNA data matrix for 99 samples that was used to determine if associations with overall survival discovered on the 291-sample discovery data generalizes to the 99-sample test data that was unseen during discovery.

A MMDS composed of global mRNA, microRNA and DNA methylation data obtained from 291 ovarian tumors resected from patients with stage 3 disease were downloaded from TCGA and jointly analyzed using JAMMIT. The goal was to determine if MMSIG exist that distinguished subtypes of ovarian cancer that lead to different clinical outcomes. Leave-one-out cross-validation (LOOCV) based on JAMMIT was applied to D to identify a MMSIG for ovarian cancer that was robust to minus-one perturbations of the 291-sample discovery data set. First, a sequence of FDR values for a monotonically increasing sequence of l1 penalty values was computed based on the JAMMIT analysis of 100 permuted versions of the super-matrix, (Methods section). An l1 penalty parameter of λ₂₉₁=0.002875 was selected based on an FDR of 0.0034619 that was a local minimum, which resulted in an mRNA signature ζ_{mRNA}⁽⁰⁾ composed of 643 genes, a miRNA signature ζ_{mRNA}⁽⁰⁾ composed of 368 microRNAs with a FDR of 0.19912, a methylation signature ζ_{miRNA}⁽⁰⁾ composed of 450 methylation loci with a FDR of 0.03038, and a MMSIG ζ⁽⁰⁾ composed of 1461 mRNA, miRNA and methylation variables that were “stacked” in the order of the Dₖ's in with a “total” FDR of 0.067647 (See Table 2).

TABLE 2
FDR profile of JAMMIT analysis of multi-modal data for ovarian cancer from TCGA

| | | | | | | (7) | | | |
|---|---|---|---|---|---|---|---|---|---|
| | | (3) | | (5) | | # of | | (9) | |
| (1) | (2) | # of | (4) | # of | (6) | selected | (8) | Total # | (10) |
| Row | l1 penalty | selected | FDR | selected | FDR | methylation | FDR | of selected | FDR |
| number | λ | mRNAs | (mRNA) | miRNAs | (miRNA) | loci | (meth)) | variables | (total) |
| 1 | 0.001 | 6081 | 0.073801 | 497 | 0.46576 | 3000 | 0.2213 | 9578 | 0.1432 |
| 2 | 0.0011042 | 5408 | 0.058633 | 485 | 0.45275 | 2701 | 0.20162 | 8594 | 0.1289 |
| 3 | 0.0012083 | 4806 | 0.047401 | 471 | 0.42646 | 2450 | 0.17776 | 7727 | 0.11503 |
| 4 | 0.0013125 | 4249 | 0.039479 | 461 | 0.4118 | 2213 | 0.16372 | 6923 | 0.1074 |
| 5 | 0.0014167 | 3766 | 0.032858 | 455 | 0.38716 | 2036 | 0.14376 | 6257 | 0.098243 |
| 6 | 0.0015208 | 3372 | 0.026972 | 451 | 0.35987 | 1841 | 0.12738 | 5664 | 0.089743 |
| 7 | 0.001625 | 3000 | 0.02226 | 442 | 0.34352 | 1662 | 0.11666 | 5104 | 0.084613 |
| 8 | 0.0017292 | 2681 | 0.018874 | 439 | 0.32977 | 1516 | 0.10182 | 4636 | 0.079444 |
| 9 | 0.0018333 | 2387 | 0.016503 | 428 | 0.30996 | 1385 | 0.09317 | 4200 | 0.075755 |
| 10 | 0.0019375 | 2124 | 0.014017 | 416 | 0.29888 | 1256 | 0.08454 | 3796 | 0.072804 |
| 11 | 0.0020417 | 1868 | 0.012385 | 412 | 0.27995 | 1119 | 0.07504 | 3399 | 0.069847 |
| 12 | 0.0021458 | 1626 | 0.0099987 | 405 | 0.27814 | 1015 | 0.06824 | 3046 | 0.069875 |
| 13 | 0.00225 | 1451 | 0.0082733 | 398 | 0.26131 | 921 | 0.06123 | 2770 | 0.067138 |
| 14 | 0.0023542 | 1289 | 0.0073703 | 391 | 0.2531 | 830 | 0.05229 | 2510 | 0.065662 |
| 15 | 0.0024583 | 1131 | 0.0068184 | 388 | 0.23449 | 734 | 0.05003 | 2253 | 0.065395 |
| 16 | 0.0025625 | 1004 | 0.0055825 | 385 | 0.22469 | 654 | 0.04387 | 2043 | 0.064688 |
| 17 | 0.0026667 | 877 | 0.0050764 | 380 | 0.21641 | 576 | 0.03767 | 1833 | 0.06503 |
| 18 | 0.0027708 | 755 | 0.0047384 | 375 | 0.20567 | 511 | 0.03158 | 1641 | 0.065201 |
| 19 | 0.002875 | 643 | 0.0034619 | 368 | 0.19912 | 450 | 0.03038 | 1461 | 0.067647 |
| 20 | 0.0029792 | 552 | 0.0036006 | 358 | 0.18865 | 382 | 0.03027 | 1292 | 0.069655 |
| 21 | 0.0030833 | 468 | 0.002718 | 355 | 0.17477 | 339 | 0.02638 | 1162 | 0.069229 |
| 22 | 0.0031875 | 394 | 0.0028249 | 343 | 0.17364 | 294 | 0.02595 | 1031 | 0.073874 |
| 23 | 0.0032917 | 342 | 0.0022084 | 336 | 0.17122 | 257 | 0.02211 | 935 | 0.076543 |
| 24 | 0.0033958 | 295 | 0.0018865 | 332 | 0.1599 | 222 | 0.02041 | 849 | 0.076782 |
| 25 | 0.0035 | 260 | 0.0022933 | 329 | 0.14848 | 186 | 0.01838 | 775 | 0.076545 |

Table 2 summarizes the relationship between l₁ penalties and FDR that is estimated based on 100 permutations of the super-matrix of a MMDS for ovarian cancer that integrates whole-genome mRNA, miRNA and DNA methylation data obtained from 291 patients with stage 3 disease. Note the FDR profiles for each data type (columns 4, 6, and 8) are decreasing towards smaller values indicating that all 3 data types contribute to some degree to a “sparse” linear model of the SOI with mRNA contributing the most in terms of FDR. In particular, row 19 (highlighted in red) is highlighted since it corresponds to a FDR for mRNA of 0.0034619 that is a local minimum of column 4. This FDR value is associated with an l₁ penalty of 0.002875 that results in a mRNA signature composed of 643 genes (FDR=0.0034619), a miRNA signature of 368 miRNAs (FDR=0.19912), a methylation signature of 450 methylation loci (FDR=0.03038), and a multi-modal signature composed of a 1461 variables (FDR=0067647).

FIG. 14 shows exemplary plots showing mRNA detector U₁ and signal of interest V₁ for ovarian cancer. Non-zero coefficients of U₁ correspond to rows of the data matrix D that best explain V₁. Only 643 out of 16020 genes contributed significantly to explaining V₁ as a sparse, linear model.

For the LOOCV analysis, the jth column of each Dₖ of D was removed to obtain minus one MMDSs, D^{(j)}={Dₖ^{(j)}}ₖ₌₁³, and minus-one stacks, D^{(j)}=stack (D^{(j)}) for j=1, 2, . . . , 291. JAMMIT was then applied to each D⁽ⁱ⁾ with λ₂₉₁=0.002875, which resulted in sⱼ-dimensional, minus-one MMSIGs, ζ^{(j)}, for j=1, 2, . . . , 291. On average, each ζ^{(j)} recapitulated 98% of the so variables of ζ⁽⁰⁾ over all 291 minus-one analyses implying that JAMMIT-derived signatures based on λ=λ₂₉₁ are robust to minus-one perturbations of the discovery data set. A single MMSIG defined by

$$ζ = {⋂}_{j} {ζ}^{( j )}$$

was generated, which contained sub-signatures composed of 534 mRNAs (ζ₁), 337 microRNAs (ζ₂) and 357 methylation loci (ζ₃) that were common to all 291 minus-one MMSIGs.

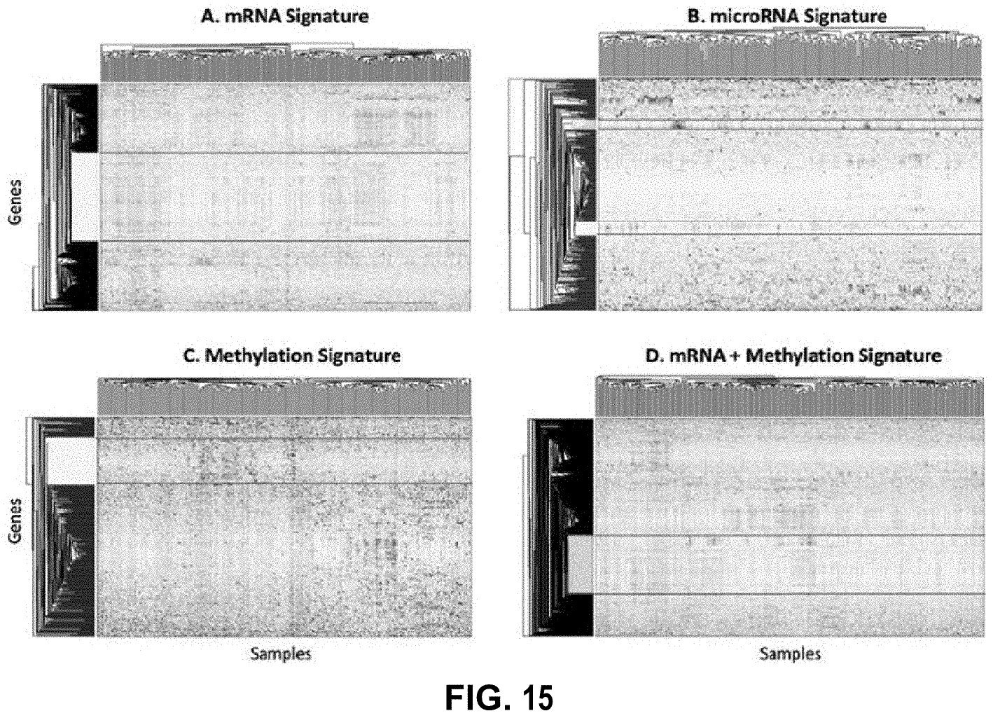

Each type-specific obtained by JAMMIT was analyzed individually and in various combinations using hierarchical cluster analysis to identify “metagenes”, i.e., subsets of variables that exhibited coordinated, low-frequency variation of expression over the 291 samples of the discovery data set. Such coherent variation offers the best opportunity to identify novel, low-dimensional signatures that capture important biological and/or clinical attributes of the tumor samples. FIG. 15 shows hierarchically clustered heatmaps of the three type-specific signatures ζ₁, ζ₂, ζ₃ for mRNA, microRNA and methylation, respectively, and a MMSIG, ζ₁₃, that “stacked” the mRNA and methylation signature.

The subscript “13” denotes the concatenation of the mRNA (1) and methylation (3) signatures derived by JAMMIT. This particular combination was chosen because the FDR values for ζ₁⁽⁰⁾ and ζ₃⁽⁰⁾ were small compared to ζ⁽⁰⁾, which implied the type-specific signatures and best explained the common SOI shared by all three different data types. Visual examination of FIG. 15, panels (A)-(C) shows that the clustered heatmaps for each type-specific signature contained meta-variables composed of variables that exhibited coordinated patterns of variation, some of which are highlighted in yellow or green. In particular, the clustered heatmap for ζ₁₃ in FIG. 15, panel (D) contained a metagene, γ, (highlighted in green) that defined a MMSIG composed of 249 variables of which 209 were mRNAs (γ₁), and 40 were methylation loci (γ₃).

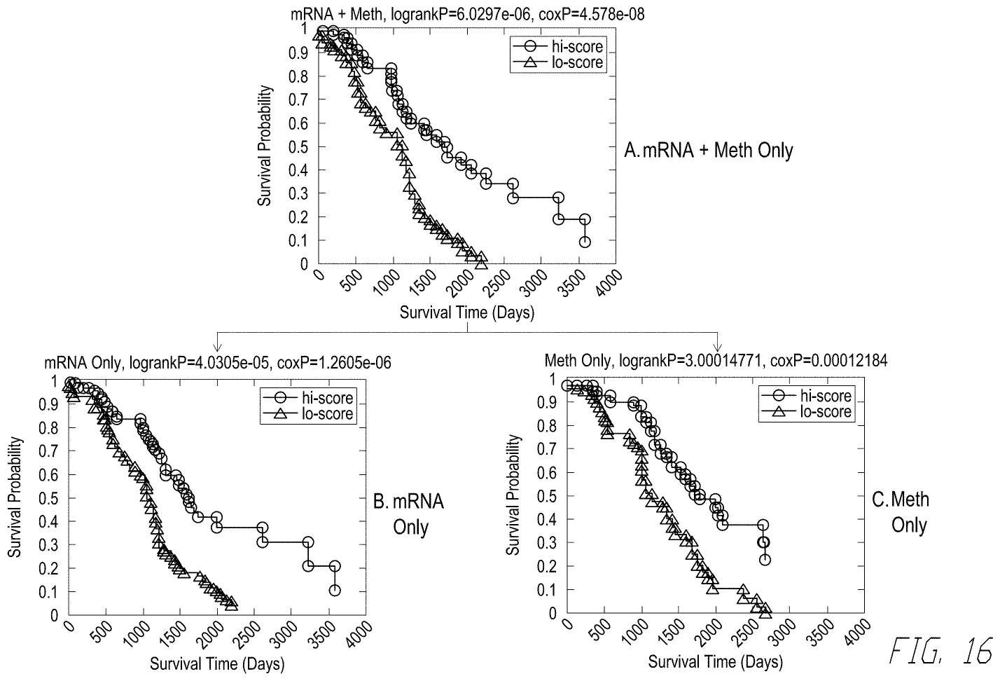

FIG. 16 shows that MMSIG, γ, and the type-specific sub-signatures, γ₁ and γ₃ were all significantly associated with overall survival on the 291 discovery samples contained in Sₙ. Interestingly, the signature composed of both mRNA and methylation variables had a more significant association with survival than signatures that contained only mRNA or only methylation variables based on log-rank and Cox regression p-values, median survival time, and 5-year survival rate.

To further reduce signature dimensionality and to better understand the biology that underlay the association of γ with overall survival, subsequent downstream analysis and interpretation can be focused on the 209-gene mRNA signature, γ₁, using IPA. In particular, the Upstream Regulator Analysis (URA) feature in IPA was used to identify sub-signatures of γ₁ that were “anchored” upstream by a single regulating molecule. Table 3 shows that Interleukin 4 (IL4) was the top upstream regulator of γ₁ that directly targeted 40 genes (out of 209) in the signature (Score=2.115 p=2.11E-20). Note that activation scores greater than 2.0 and p-values less than 1.0E-03 are considered significant. The 40 genes in γ₁ directly targeted by IL4 were used to define a mRNA signature φ_{IL4}⁽⁴⁰⁾ contained in γ₁ that was “anchored” upstream by IL4.

TABLE 3
Top Upstream Regulators of mRNA signature γ1 for ovarian cancer

| Upstream | Predicted | Activation | Intersection P- | Number of |
|---|---|---|---|---|
| Regulator | State | Score | value | Targets |
| IL4 | Activated | 2.115 | 2.115E−20 | 40 |
| OSM | Activated | 2.616 | 2.41E−08 | 21 |
| Stat5(A/B) | Activated | 2.630 | 6.50E−08 | 9 |

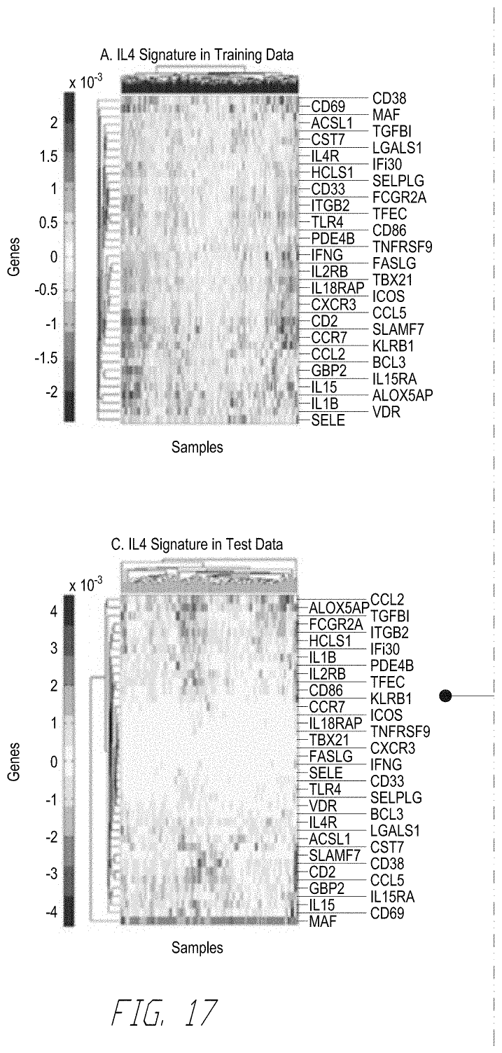

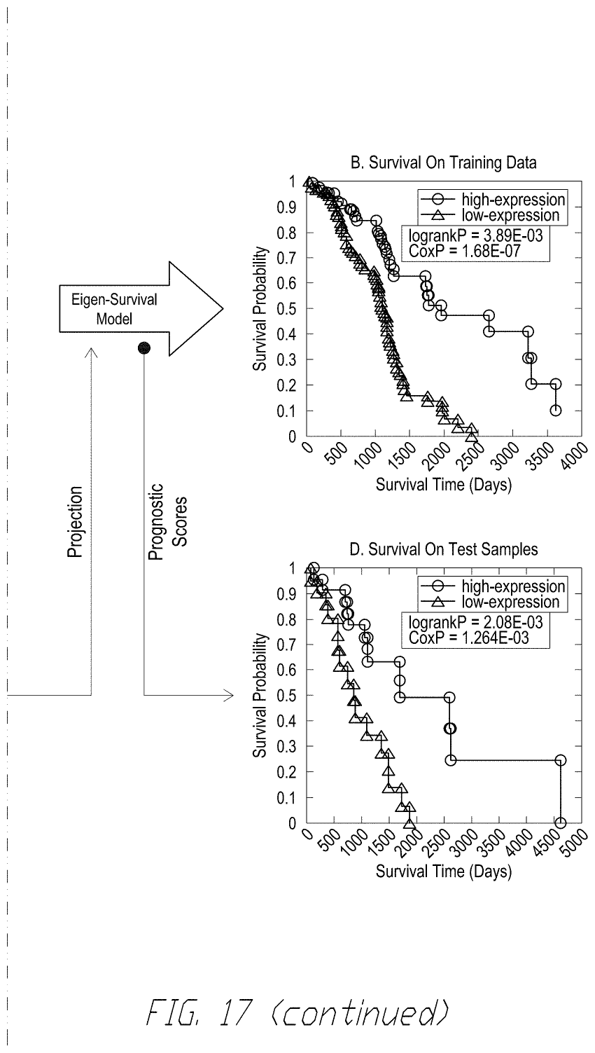

FIG. 17 shows the results of an eigen-survival analysis based on the realization of φ_{IL4}⁽⁴⁰⁾ in the expression data for the 291 patients in the discovery data set. FIG. 17, panel (A) shows the clustered heatmap of φ_{IL4}⁽⁴⁰⁾ realized in the training data set and Figure, panel (B) shows KM plots based on prognostic scores for each patient derived from the ESM extracted from the expression patterns in FIG. 17, panel (A). In FIG. 17, panel (B), 144 patients with prognostic scores in the top and bottom quartiles have significantly different KM plots with log-rank p-value of 3.89E-06 (log rankP). Moreover, a Cox regression model of overall survival based on prognostic scores for all 291 patients with age as a covariate had a p-value of 1.68E-07 (CoxP), which provides further validation of the eigen-survival model derived from expression patterns visualized in FIG. 17, panel (A). FIG. 17, panel (C) shows the clustered heatmap of the φ_{IL4}⁽⁴⁰⁾ signature realized in whole-genome mRNA data for 99 independent test tumor samples. The prognostic scores for the 99 test patients were computed by processing the expression patterns in FIG. 17, panel (C) using the ESM derived from the expression patterns in FIG. 17, panel (A). FIG. 17, panel (D) shows that test patients with prognostic scores in the top and bottom quartiles have significantly different survival statistics (log rankP=2.08E-03, CoxP=1.26E-03). Hence, the ESM based on φ_{IL4}⁽⁴⁰⁾ captured information related to overall survival that was also applicable to the 99-samples of the independent test data set that were unseen during discovery.

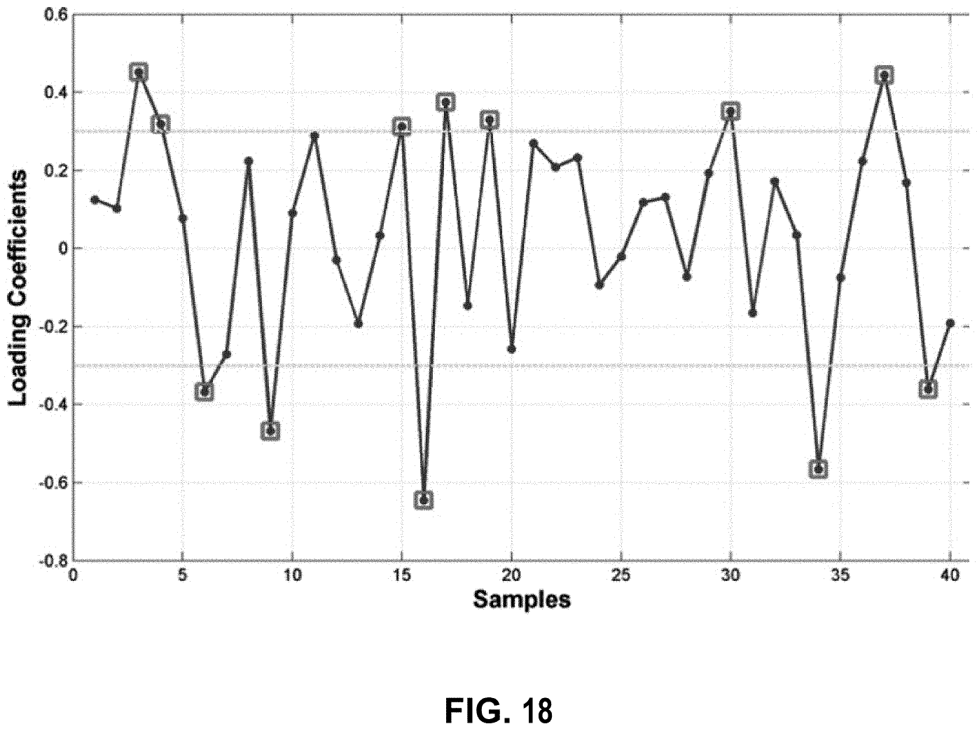

The dimensionality of φ_{IL4}⁽⁴⁰⁾ can be further reduced based on the ESM extracted from the 291 discovery samples. FIG. 18 shows a plot of the 40 loading coefficients associated with the ESM derived from expression patterns in FIG. 17, panel (A) with 12 high magnitude coefficients highlighted in red. The 12 genes corresponding to these coefficients were assembled to form the mRNA signature, φ_{IL4}⁽¹²⁾, that was tested for association with overall survival on the 291-sample discovery data set and the 99-sample independent test data set.

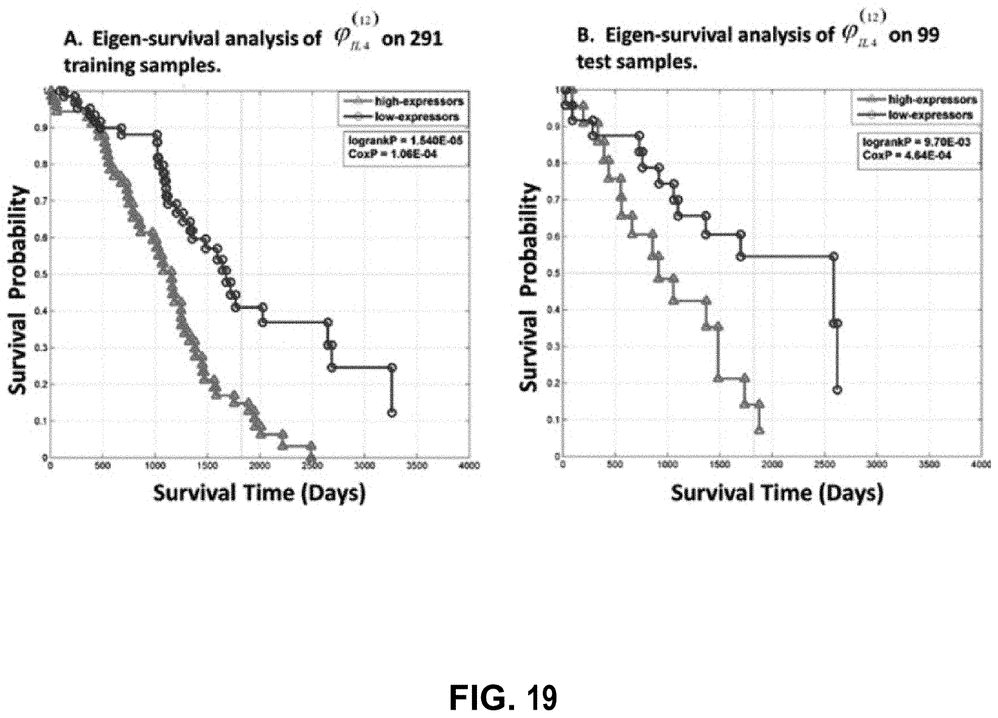

FIG. 19, panel (A) shows that ESM based on φ_{IL4}⁽¹²⁾ in the 291 samples of the discovery data set was significantly associated with overall survival (log rankP=1.54E-05, CoxP=1.06E-04). Moreover, FIG. 19, panel (B) shows that the ESM based on φ_{IL4}⁽¹²⁾ realized in the discovery data generalizes to the 99 samples of the independent test data set (log rankP=9.70E-03, CoxP=4.64E-04). Interestingly, the set of 28 genes in φ_{IL4}⁽⁴⁰⁾ complementary to the genes in φ_{IL4}⁽¹²⁾ failed to generalize on the 99 independent test samples. These results validate the BEST principle as implemented by JAMMIT for the joint analysis of multiple data sets in ovarian cancer.

Note that IL4 directly targets every gene in φ_{IL4}⁽¹²⁾ per IPA. Studies show the IL4 induces the transformation of Tumor Associated Macrophages (TAMs) that infiltrate the tumor microenvironment into the M2 phenotype, which confers a survival advantage to cancer cells and promotes tumor growth. An alternative pathway involving Interferon Gamma (IFNG) and Tumor Necrosis Factor Alpha (TNFA) transform TAMs into the M1 phenotype that exerts a cytotoxic effect on genetically mutated cancer cells. It has been reported that a high M1/M2 ratio is associated with extended survival in ovarian cancer patients. This suggests that immune cell polarization in the tumor microenvironment impacts overall survival of patients with ovarian cancer undergoing standard platinum-based chemotherapy combined with paclitaxel. Indeed, the φ_{IL4}⁽¹²⁾ signature contains the Chemokine (C-C motif) Ligand 2 (CCL2) gene, which is a chemokine that recruits monocytes from the bloodstream to the tumor microenvironment. It has been reported that CCL2 is up-regulated in ovarian cancer and the blockade of CCL2 protein expression enhances chemotherapeutic response.

Immune Checkpoint Blockade.

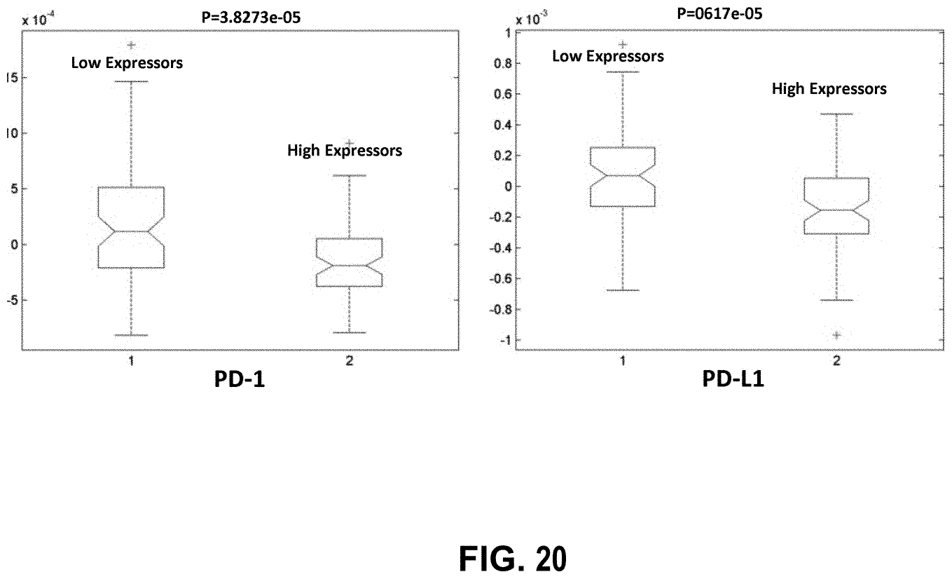

FIG. 20 shows that checkpoint genes are differentially expressed between good and poor responders to chemotherapy per the IL4 signature. Negative feedback mechanism can modulate immune cell response in the tumor microenvironment. A cancer patient can be “cured” of metastatic melanoma (to brain and liver) after treatment with anti-PD-L1 antibody Keytruda. However, such treatments may only works on a fraction of patients. PD-L1 is poor predictor of response to treatment. More accurate biomarkers are needed. The IL4 signature may be a predictive of response to treatment that combines platinum-based chemotherapy and immune checkpoint blockade. Other check point genes significantly up-regulated in good responders relative to poor responders identified by the IL4 signature included PD-1, PD-L1, CTLA4, LAGS, ICOS, and INDO.

Altogether, these data demonstrate that multi-modal signatures composed of mRNA and methylation variables can result in better predictive models of overall survival than uni-modal signatures composed of only mRNA or DNA methylation variables alone.

### Example 3

### Imaging Genomics of Liver Cancer

This example describes JAMMIT analysis of whole-genome mRNA and PET imaging data for liver cancer.

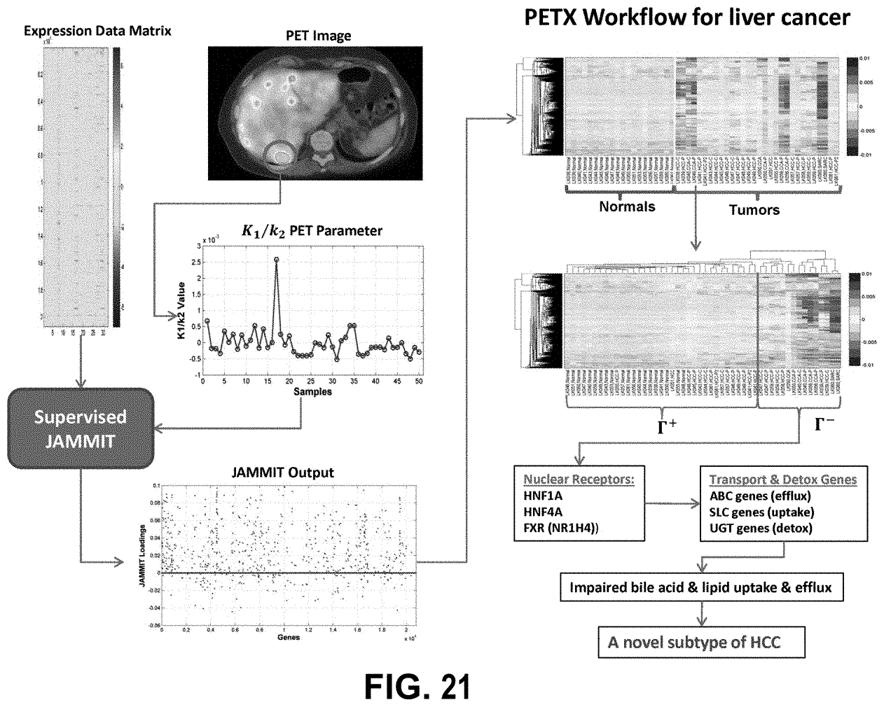

FIG. 21 outlines JAMMIT analysis of whole-genome mRNA and PET imaging data for liver cancer. Twenty patients referred for surgical resection of liver tumors were prospectively recruited to participate in an institutional review-board approved clinical research study with written informed consent. Prior to surgery, these patients underwent liver imaging with a Philips Gemini TF-64 PET/CT scanner (Philips Healthcare, Andover, Mass.) using 18F-fluorocholine under an investigational new drug protocol. In a previous single-institution clinical trial, 18F-fluorocholine, a tracer of choline phospholipid synthesis, affords PET/CT with relatively high diagnostic sensitivity for HCCs. Presently, less is known regarding the diagnostic utility of 18F-fluorocholine for ICCs and other sub-types of liver cancer. Regions of interest (ROI) analysis of the PET/CT images were used to generate time activity curves corresponding to the 1) arterial pool in the descending aorta and 2) areas of tissue within the liver that corresponded to the tumor and adjacent liver samples profiled by expression arrays. PET kinetic analysis was then applied based on a 2-tissue compartment model (2TC) of 18F-fluorocholine pharmacokinetics in liver tumor and liver tissue. Pharmacokinetic parameters K₁, k₂, k₃, k₄, K₁/k₂, and Flux for each 2TC model corresponding to each sample were estimated using PMOD 3.4 (PMOD Technologies, Zurich Switzerland) and assembled to form a 6×50 Pet kinetics data matrix for the 50 tissue samples included in the experiment.

Tumor and adjacent non-tumor liver tissue specimens were obtained subsequently during surgery, and RNA was extracted from homogenized frozen tissue lysates in RLT Plus buffer with the AllPrep DNA/RNA Mini kit (Qiagen, Valencia, Calif.) following manufacturer's protocol. The isolated RNA was stored at −80° 0 until used. The quality of the total RNAs was checked on a Bioanalyzer using RNA 6000 Nano chips (Agilent, Santa Clara, Calif.). The RNA samples were processed following the WG-DASL assay protocol (Illumina Inc., Sunnyvale, Calif.) and the resulting PCR products were hybridized onto the Illumina HumanHT-12 v4 Expression BeadChips included over 24,000 transcripts with genome-wide coverage of well-characterized genes, gene candidates, and splice variants. Arrays were scanned using the iScan™ instrument and expression levels were quantified using GenomeStudio software (IIlumina Inc., Sunnyvale, Calif.).

Gene-level expression values were assembled to form a 20792×50 data matrix where the rows represented 20792 genes and columns represented 50 adjacent-normal and tumor samples obtained from 20 patients. Columns 1-20 of the data matrix represented adjacent-normal samples while columns 21-50 represented 30 liver tumors of which 22 were hepatocellular carcinomas (HCCs), 6 were intra-hepatic cholangiocarcinomas (ICC) and 2 were sarcomas. The data matrix was pre-processed by generalized log 2 transformation with background subtraction, quantile normalization, and row centering.

Whole-genome expression data were collected for 20792 genes in 20 adjacent-normal, 22 hepatocellular carcinoma (HCC), 6 intra-hepatic cholangiocarcinoma (ICC) and 2 sarcoma samples using DASL microarrays. The expression data were assembled to form a 20792×50 raw expression data matrix where columns 1-20 represented the normal samples and columns 21-50 represented the tumor samples. The data matrix of raw expression was pre-processed by the generalized log 2 transform, quantile normalization and row-centering to obtain the pre-processed expression data matrix H_{mRNA}. The values of six kinetic parameters, K₁, k₂, k₃, k₄, K₁/k₂/, lux obtained from 2TC models for each tissue sample formed the columns of a 6×50 data matrix that was row-centered to obtain the PET data matrix, H_{PET}. A final pre-processing step involved the scaling of the stacked matrix H_{PETX}=StaCk(H_{mRNA}, H_{PET}) by its Frobenius norm. The goal of this analysis is to identify mRNA signatures that are highly correlated with the PET kinetic parameters.

Six different analyses of H_{mRNA} based on JAMMIT were conducted where each analysis was supervised by a single PET kinetic parameter. That is, JAMMIT was applied to H_{PETX}^{(l)}={H_{mRNA},H_{PET}^{(l)}} where H_{PETX}^{(l)} is a 1-dimensional vector equal to the lth row of H_{PET} for 1=1, 2, . . . , 6. Of the six possible analyses, only supervision by the H_{PETX}⁽⁵⁾=K₁/k₂ kinetic parameter resulted in a FDR profile that implied significant joint correlations between H_{mRNA} and H_{PET} (see Table 4).

TABLE 4
FDR profile of K₁/k₂ signature for liver cancer

| | | (3) | | (5) | | (7) | |
|---|---|---|---|---|---|---|---|
| | (2) | # of | (4) | # of | (6) | # of | (8) |
| Row | l₁ penalty | selected | FDR | selected | FDR | selected | FDR |
| Number | λ | genes | (genes) | PET parms | (PET) | variables | (total) |
| 1 | 1.00E−05 | 20613 | 0.98581 | 1 | 0 | 20614 | 0.98577 |
| 2 | 0.00082208 | 12393 | 0.364 | 1 | 0 | 12394 | 0.36403 |
| 3 | 0.0016342 | 8270 | 0.16171 | 1 | 0 | 8271 | 0.1618 |
| 4 | 0.0024463 | 5682 | 0.085654 | 1 | 0 | 5683 | 0.085805 |
| 5 | 0.0032583 | 4009 | 0.047712 | 1 | 0 | 4010 | 0.047936 |
| 6 | 0.0040704 | 2943 | 0.026501 | 1 | 0 | 2944 | 0.026829 |
| 7 | 0.0048825 | 2215 | 0.014121 | 1 | 0 | 2216 | 0.01455 |
| 8 | 0.0056946 | 1696 | 0.0074933 | 1 | 0 | 1697 | 0.0080397 |
| 9 | 0.0065067 | 1322 | 0.0041554 | 1 | 0 | 1323 | 0.0048742 |
| 10 | 0.0073187 | 1052 | 0.0020717 | 1 | 0 | 1053 | 0.0029486 |
| 11 | 0.0081308 | 822 | 0.0010654 | 1 | 0 | 823 | 0.0022007 |
| 12 | 0.0089429 | 652 | 0.00054949 | 1 | 0 | 653 | 0.0018897 |
| 13 | 0.009755 | 534 | 0.00026091 | 1 | 0 | 535 | 0.0019159 |
| 14 | 0.010567 | 442 | 0.00038277 | 1 | 0 | 443 | 0.002179 |
| 15 | 0.011379 | 373 | 0.00021345 | 1 | 0 | 374 | 0.0024213 |
| 16 | 0.012191 | 327 | 0.00012174 | 1 | 0 | 328 | 0.0024575 |
| 17 | 0.013003 | 279 | 3.57E−05 | 1 | 0 | 280 | 0.00263 |
| 18 | 0.013815 | 226 | 0 | 1 | 0 | 227 | 0.0032441 |
| 19 | 0.014628 | 198 | 0 | 1 | 0 | 199 | 0.0034505 |
| 20 | 0.01544 | 165 | 0 | 1 | 0 | 166 | 0.0037168 |
| 21 | 0.016252 | 133 | 0 | 1 | 0 | 134 | 0.0046787 |
| 22 | 0.017064 | 104 | 0 | 1 | 0 | 105 | 0.0058761 |
| 23 | 0.017876 | 82 | 0 | 0 | 1 | 82 | 0.0093446 |
| 24 | 0.018688 | 58 | 0 | 0 | 1 | 58 | 0.012182 |
| 25 | 0.0195 | 43 | 0 | 0 | 1 | 43 | 0.017589 |

Table 4 shows FDR profile of JAMMIT analysis of whole-genome expression data supervised by the K₁/k₂ PET parameter for liver cancer. Note the K₁/k₂ PET parameter (column 5) is selected for inclusion in the sparse model of the SOI for most l₁ penalties with FDR values of zero. Moreover, the FDR profile for genes (column 4) is rapidly decreasing indicating a strong signature for gene expression. These results taken together suggest that the K₁/k₂ parameter is associated with gene expression via the sparse linear model for the SOI. In particular, row 12 (highlighted in red) corresponds to a FDR for mRNA of 0.00054949 that is a local minimum of column 4. This FDR value is associated with an l₁ penalty of 0.0089429 that results in a mRNA signature composed of 652 genes.

A locally minimal FDR*=0.000549 was selected from the FDR profile for genes that corresponded to an l₁ penalty parameter value of λ*=0.0089429. A JAMMIT analysis based on this value of λ resulted in a mRNA signature containing 652 genes that was significantly correlated with the K₁/k₂ kinetic parameter, which can be denoted as ω_{mRNA}^{(K}^{₁}^{/k}^{₂}⁾(H_{mRNA}), and the K₁/k₂ PET parameter were significantly correlated (r=0.413, p=0.00293). In sharp contrast, the FDR profile for a JAMMIT analysis of H_{mRNA} supervised by other PET kinetic parameters, including the K₁ kinetic parameter failed to produce an l₁ penalty that correlated the two data types (see Table 5).

TABLE 5
FDR profile for K₁ signature for liver cancer

| | | | | (5) | | (7) | |
|---|---|---|---|---|---|---|---|
| | | (3) | | # of | | # of | |
| (1) | | # of | (4) | Selected | (6) | Selected | (8) |
| Row | (2) | Selected | FDR | PET | FDR | Variables | FDR |
| Number | alty | Genes | (Genes) | Params | (PET) | (Total) | (Total) |
| 1 | 1.00E−05 | 20609 | 0.98622 | 1 | 0 | 20610 | 0.98617 |
| 2 | 0.00082208 | 12395 | 0.38683 | 0 | 1 | 12395 | 0.38682 |
| 3 | 0.0016342 | 8279 | 0.16905 | 0 | 1 | 8279 | 0.16904 |
| 4 | 0.0024463 | 5687 | 0.079955 | 0 | 1 | 5687 | 0.079951 |
| 5 | 0.0032583 | 4017 | 0.039391 | 0 | 1 | 4017 | 0.03939 |
| 6 | 0.0040704 | 2946 | 0.020025 | 0 | 1 | 2946 | 0.020024 |
| 7 | 0.0048825 | 2216 | 0.010886 | 0 | 1 | 2216 | 0.010885 |
| 8 | 0.0056946 | 1697 | 0.0062515 | 0 | 1 | 1697 | 0.0062512 |
| 9 | 0.0065067 | 1325 | 0.0038756 | 0 | 1 | 1325 | 0.0038754 |
| 10 | 0.0073187 | 1054 | 0.0025682 | 0 | 1 | 1054 | 0.0025681 |
| 11 | 0.0081308 | 823 | 0.0021887 | 0 | 1 | 823 | 0.0021886 |
| 12 | 0.0089429 | 653 | 0.0016764 | 0 | 1 | 653 | 0.0016764 |
| 13 | 0.009755 | 535 | 0.0015997 | 0 | 1 | 535 | 0.0015997 |
| 14 | 0.010567 | 442 | 0.0012158 | 0 | 1 | 442 | 0.0012158 |
| 15 | 0.011379 | 375 | 0.0010085 | 0 | 1 | 375 | 0.0010084 |
| 16 | 0.012191 | 327 | 0.00051738 | 0 | 1 | 327 | 0.00051735 |
| 17 | 0.013003 | 279 | 0.00049938 | 0 | 1 | 279 | 0.00049935 |
| 18 | 0.013815 | 228 | 0.00021824 | 0 | 1 | 228 | 0.00021823 |
| 19 | 0.014628 | 198 | 5.03E−05 | 0 | 1 | 198 | 5.03E−05 |
| 20 | 0.01544 | 165 | 6.03E−05 | 0 | 1 | 165 | 6.03E−05 |
| 21 | 0.016252 | 133 | 0 | 0 | 1 | 133 | 0 |
| 22 | 0.017064 | 105 | 0 | 0 | 1 | 105 | 0 |
| 23 | 0.017876 | 82 | 0 | 0 | 1 | 82 | 0 |
| 24 | 0.018688 | 58 | 0 | 0 | 1 | 58 | 0 |
| 25 | 0.0195 | 43 | 0 | 0 | 1 | 43 | 0 |

Table 5 shows a FDR profile of JAMMIT analysis of whole-genome expression data supervised by the K₁ PET pharmacokinetic parameter for liver cancer. This FDR profile indicates a lack of correlation between global gene expression and the K₁ PET kinetic parameter. Note that the K₁ PET parameter (column 5) is NOT selected for inclusion in the model of the SOI for all but the first l₁ penalty value (see row 1) with FDR values of 1.0. This result is in sharp contrast to the FDR profile for gene expression (column 4) where the FDR values rapidly decrease to small values. This result suggests that although there is a strong coherent signature for gene expression that contributes to the common SOI, this signal is not correlated with the K₁ PET parameter.

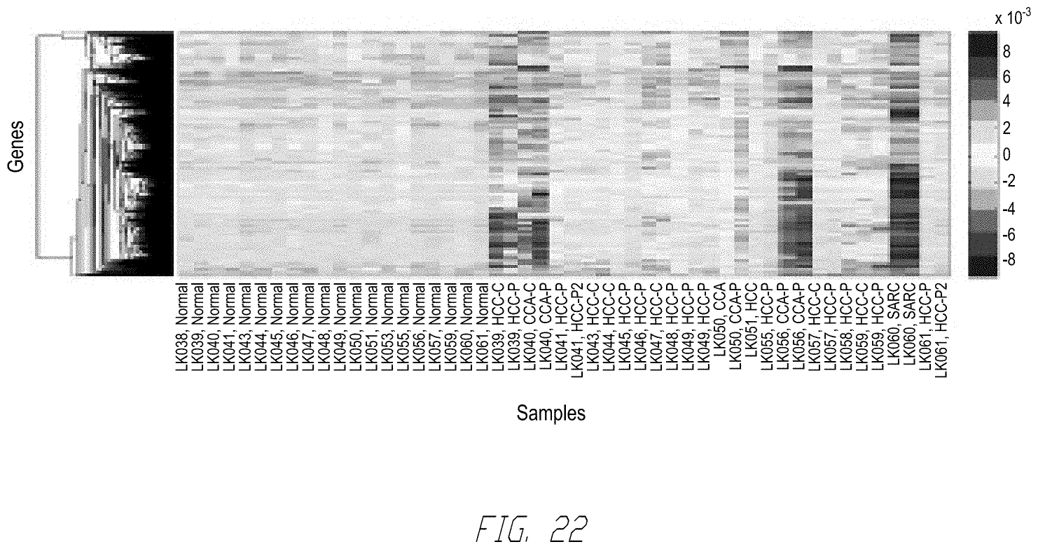

FIG. 22 visualizes the realization of ω_{mRNA}^{(K}^{₁}^{/k}^{₂}⁾ in of H_{mRNA} as a row-clustered heatmap where aggregate gene expression is highly variable on the tumor samples (columns 21-50) compared to the normal samples (columns 1-20).

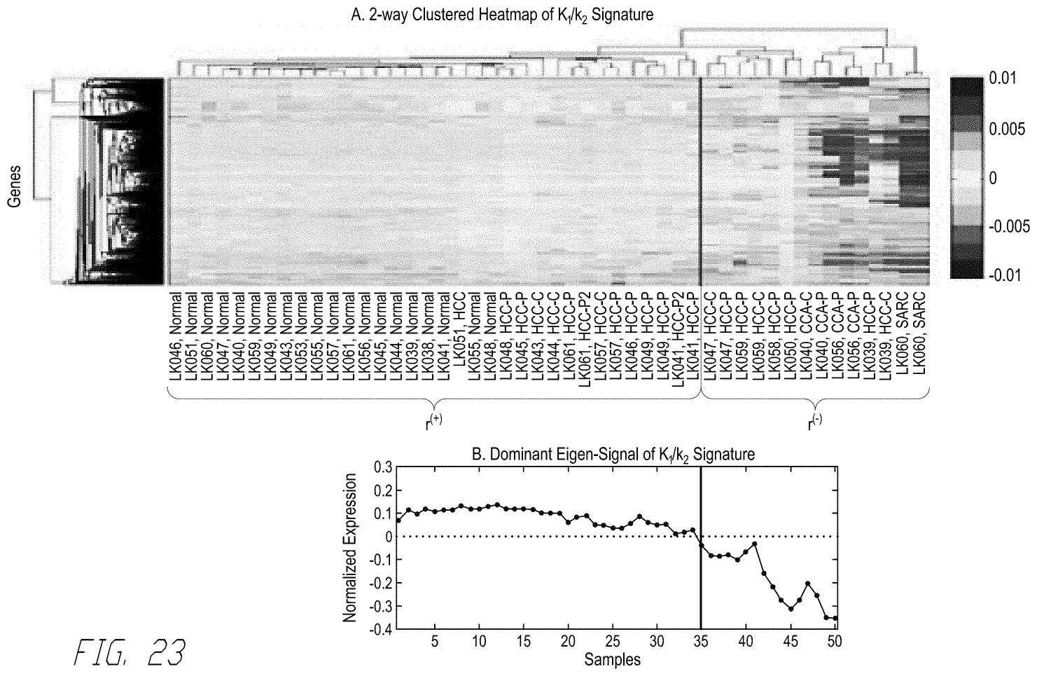

FIG. 23, panel (A) shows a 2-way clustered heatmap of ω_{mRNA}^{(K}^{₁}^{/k}^{₂}⁾ and ω_{mRNA}^{(K}^{₁}^{/k}^{₂}⁾ is preferentially down-regulated on a set of 15 tumors relative to a complementary subset of fifteen (15) HCCs and twenty (20) normal samples. Let Γ⁽⁻⁾ denote the set of column indices of H_{mRNA} that correspond to the samples where ω_{mRNA}^{(K}^{₁}^{/k}^{₂}⁾ is down-regulated relative to the set of column indices, denoted by Γ⁽⁺⁾, that correspond to samples where ω_{mRNA}^{(K}^{₁}^{/k}^{₂}⁾ is up-regulated. In FIG. 23, panel (B) the dominant eigen-signal of the 2-way, clustered heatmap in FIG. 23, panel (A) clearly discriminates between the samples in Γ⁽⁻⁾ and Γ⁽⁺⁾ based on a threshold set at zero. The ability of ω_{mRNA}^{(K}^{₁}^{/k}^{₂}⁾ to discriminate between the samples in F and Γ⁽⁺⁾ suggests two distinct expression phenotypes for HCC represented by the seven (7) HCC in Γ⁽⁻⁾ and fifteen (15) HCC in Γ⁽⁺⁾. Moreover, the co-clustering of 7 HCC samples in Γ⁽⁻⁾ along with 6 ICC suggests that these HCC samples represent a cholangiocarcinoma-like HCC subtype (CCL-HCC), which may share clinical and biological attributes of this more aggressive sub-type of liver cancer.

Table 6 lists the top canonical pathways and upstream regulators of ω_{mRNA}^{(K}^{₁}^{/k}^{₂}⁾ according to IPA. The top upstream regulators included the nuclear receptors HNF4A, HNF1A, and FXR (NR1H4), and where HNF4A and HNF1A were predicted to be down-regulated with very high statistical significance. Moreover, FXR/LXR and LXR/RXR Activation were the top canonical pathways in the ω_{mRNA}^{(K}^{₁}^{/k}^{₂}⁾ signature and most of the genes in both pathways were down-regulated indicating inactivation of these pathways upstream of ω_{mRNA}^{(K}^{₁}^{/k}^{₂}⁾. The dominate downstream effects of ω_{mRNA}^{(K}^{₁}^{/k}^{₂}⁾ predicted by IPA included biological functions related to the dysregulation of lipid and bile acid metabolism as well as disease functions related to the initiation and progression of HCC and ICC. For example, the inactivation of HNF4A as a significant upstream regulator of ω_{mRNA}^{(K}^{₁}^{/k}^{₂}⁾ is consistent with published reports that HNF4A down-regulation suppresses hepatocyte differentiation and commitment to the biliary lineage in ICC and CCL-HCC. Moreover, loss of HNF1A function in hepatocytes leads to the activation of pathways involved in tumorigenesis. Studies also show reduced HNF4A and FXR expression in human HCC and ICC, and that mice lacking FXR expression spontaneously developed HCC.

TABLE 6
IPA analysis of mRNA signature ω_{mRNA}^{(K}^{₁}^{/k}^{₂}⁾ for liver cancer
Top Canonical Pathways

| Pathway | P-Value | Overlap |
|---|---|---|
| FXR/RXR Activation | 3.03E−60 | 48.8% (62/127) |
| LXR/RXR Activation | 2.36E−37 | 37.2% (45/121) |
| LPS/IL1 Mediated Inhibition | 5.89E−25 | 20.5 (45/219) |
| of RXR | | |
| Function | | |

Top Upstream Regulators

| Upstream Regulator | P-Value of | Predicted |
|---|---|---|
| HNF1A | 2.02E−78 | Inhibited |
| PPARA | 4.40E−46 | |
| HNF4A | 4.20E−44 | Inhibited |
| FXR | 1.95E−38 | |
| GW4064 | 1.85E−34 | Inhibited |

In addition, the ω_{mRNA}^{(K}^{₁}^{/k}^{₂}⁾ signature included 46 membrane transport genes from the ATP-Binding Cassette (ABC) and Solute Carrier (SLC) super-families, almost all of which were significantly down-regulated in the tumor samples of Γ⁽⁻⁾ relative to the samples in Γ⁽⁺⁾. Recall that the dominant eigen-signal of ω_{mRNA}^{(K}^{₁}^{/k}^{₂}⁾ (D₁) was found to be significantly correlated with the vector of K₁/k₂ parameter values for the 50 samples included in the study (r=0.413,p=0.00293). The K₁/k₂ parameter derived from 18F-fluorocholine PET data reflects the blood-tissue equilibrium of choline, a nutrient important for phospholipid and bile homeostasis, as well as lipid transform. Therefore, it is not surprising that the ω_{mRNA}^{(K}^{₁}^{/k}^{₂}⁾ signature contained a significant number of ABC and SLC membrane transport genes, since these genes regulate the influx and efflux of bile and lipids across the membranes of hepatocytes and cholangiocytes and are tightly regulated by nuclear receptors HNF4A, HNF1A and FXR. The herein suggests the inactivation of HNF4A, HNF1A and FXR upstream of ω_{mRNA}^{(K}^{₁}^{/k}^{₂}⁾ suppresses the uptake and efflux of bile and lipids downstream of ω_{mRNA}^{(K}^{₁}^{/k}^{₂}⁾ by down-regulating the expression of specific ABC and SLC genes that are members of ω_{mRNA}^{(K}^{₁}^{/k}^{₂}⁾. In addition to the wide-spread disruption of bile acid and lipid homeostasis, the down-regulation of membrane transporters in ω_{mRNA}^{(K}^{₁}^{/k}^{₂}⁾ directly impacts liver carcinogenesis and tumor progression. For example: i) SLC22A1 is associated with progression and survival in human intrahepatic cholangiocarcinoma; ii) knockout mice lacking ABCB4 suffer from the loss of biliary phospholipid secretion and spontaneously develop HCC; iii) transporter genes ABCB1, ABCC6, ABCC9, ABCG2 are down-regulated in prostate cancer; iv) ABCB11/BSEP (Bile Salt Export Pump) and FXR expression is reduced in HCC; and v) SLC22A1 is epigenetically silenced in human HCC.

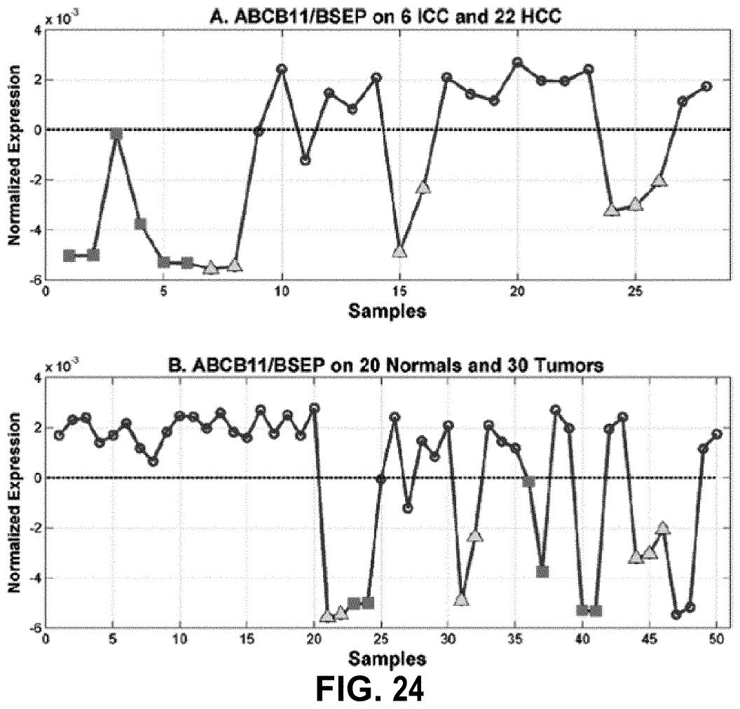

FIG. 24 shows the expression profiles of the ABCB11 gene (also known as the Bile Salt Export Pump or BSEP), in two different groupings of the samples: i) ICC vs HCC compares 6 ICC (columns 1-6) and 22 HCC (columns 7-28); and ii) NRM vs TUMOR compares 20 Normals (columns 1-20) and 30 Tumors (columns 21-50). FIG. 24, panel (A) shows that the ABCB11 gene is down-regulated in the ICC samples (red squares) and CCL-HCC samples (green triangles) relative to the HCC samples (blue circles) in the ICC vs HCC data set based on a horizontal threshold set at zero. FIG. 24, panel (B) shows that ABCB11 is uniformly up-regulated on the 20 normals and highly variable on the tumors with preferential down-regulated on the ICC (red circles), CCL-HCC (green triangles) and sarcoma samples in the NRM vs TUMOR data set. The ABCB11 gene codes for a protein that facilitates the efflux of bile acids out of the liver and defects in the ABCB11 gene result in progressive familial intrahepatic cholestasis, which is a progressive liver disease that often starts early in life and rapidly progresses to end-stage liver disease with an increased risk for HCC. The herein suggests that the ICC and CCL-HCC subtypes can be characterized in part by the suppression of bile acid efflux that is mediated by the down-regulation of the ABCB11 transporter gene.

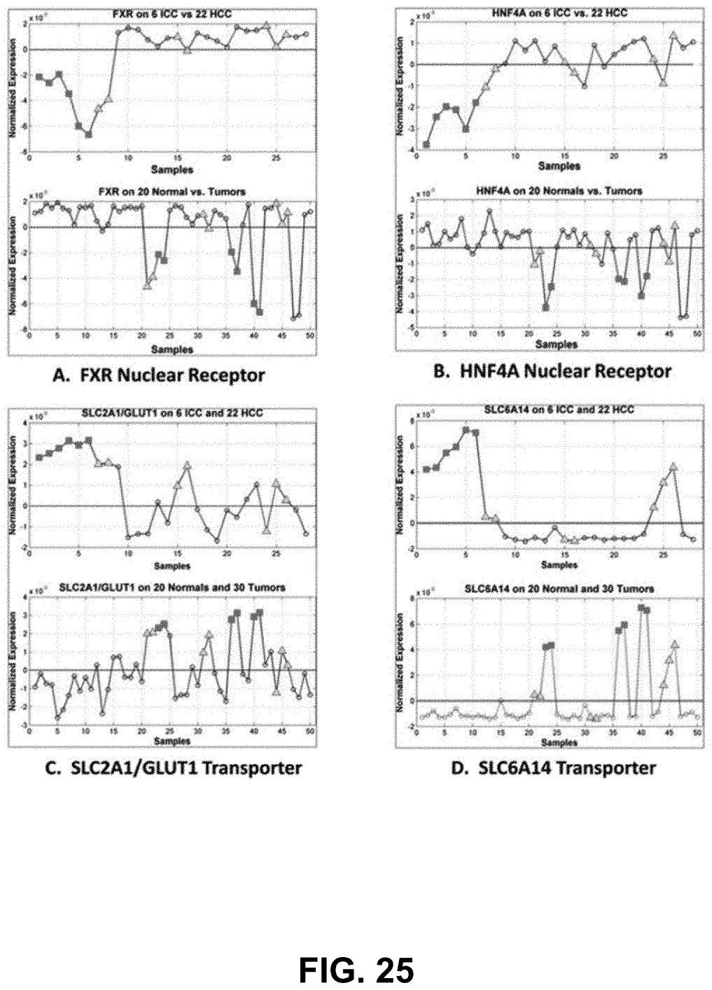

FIG. 25 shows the expression profiles of nuclear receptors FXR and HNF4A and the SLC transporter genes SLC2A1/GLUT1 and SLC6A14 in the ICCvsHCC and NRM vs TUMOR experimental designs. Panels (A) and (B) of FIG. 25 confirm that both FXR and HNF4A are preferentially down-regulated in ICCs relative to the HCC, uniformly up-regulated on the normals relative to liver tumors, and highly variable on the tumors with preferential down-regulation on the tumors in Γ⁽⁻⁾. Panel C of FIG. 25 shows that unlike the nuclear receptors FXR and HNF4A, the SLC2A1/GLUT1 transporter is up-regulated in ICC relative to HCC, uniformly down-regulated on normals relative to tumors, and highly variable on tumors but with preferential up-regulation on the tumors in Γ⁽⁻⁾. In FIG. 25, panel (D), SLC6A14 shows strikingly high and specific up-regulation on all 6 ICC and 5 of 7 CCL-HCC samples relative to the remaining 15 HCC samples in the ICCvsHCC design. Moreover, that SLC6A14 was uniformly down-regulated on the normals compared to the tumors in NRM vs TUMOR with significant up-regulation concentrated on the ICC and CCL-HCC samples. SLC6A14 is reported to be highly activated in cancers of the colon, cervix, breast, and pancreas and the blockade of SLC6A14 has been suggested as a treatment for many solid tumors. The expression profiles in FIG. 25, panel (D) supports the possibility that the SLC6A14 transporter may be therapeutic targets in ICC and CCL-HCC.

The correlation between ω_{mRNA}^{(K}^{₁}^{/k}^{₂}⁾ and the K₁/k₂ PET parameter suggests that the Γ⁽⁻⁾ and Γ⁽⁺⁾ expression phenotypes can be distinguished by K₁/k₂ parameter values. To test this hypothesis, the information content of the K₁/k₂ parameter vector can be encoded in a Generalized Regression Neural Network (GRNN) implemented in MATLAB (The MathWorks Inc., Natick, Mass.) after denoising by the Daubechies mother wavelet of order 3 over 5 scales. The GRNN model was trained using a “spread” parameter set at 0.23235 that defines the level of smoothing of the GRNN output. Training of the GRNN was supervised by a binary target vector, T∈{0,1}⁵⁰, where the samples in Γ⁽⁺⁾ and Γ⁽⁻⁾ were labeled with a “0” and “1”, respectively.

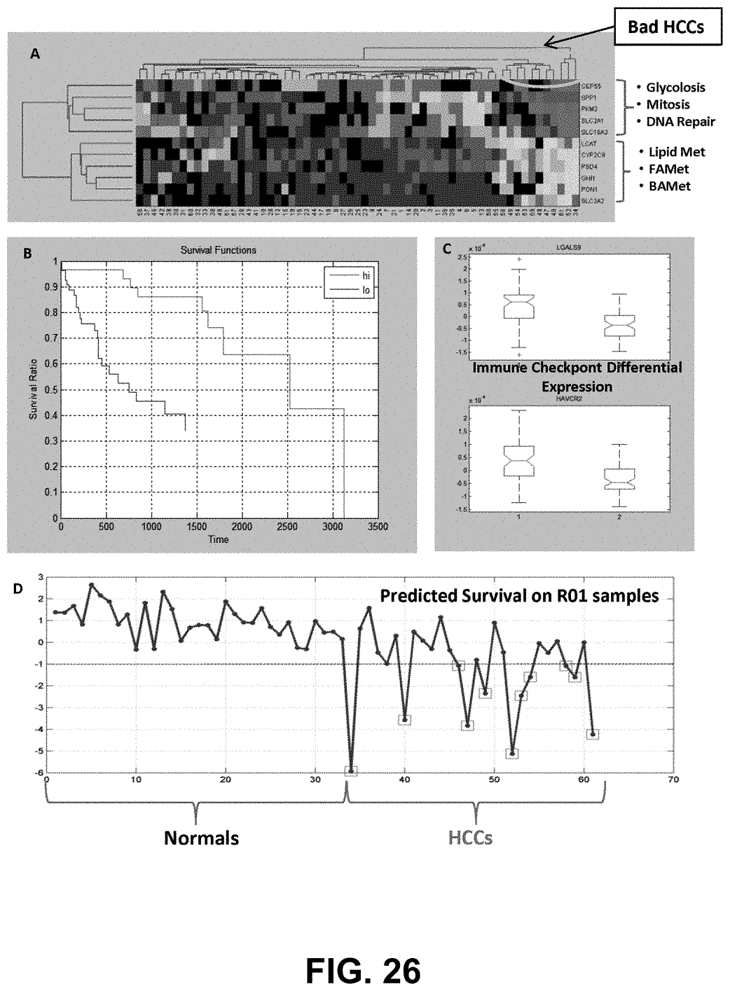

FIG. 26 shows identification of a 11-gene super-signature for liver cancer. SupSig11={CEP55, SPP1, PKM2, SLC2A1, SLC16A3, LCAT, CYP2C9, PSD4, GHR, PON1, SLC2A2}. SupSig11 is “super” because every sub-signature (including all singletons) predicts survival in TCGA. SupSig11 identifies a subset of “bad” HCCs (FIG. 26, panel (A)). SupSig11 predicts survival in TCGA (FIG. 26, panel (B)). Patients with low eigen-gene expression have significantly shorter survival than patients with high eigen-gene expression (FIG. 26, panel (B)). Immune checkpoint gene TIM3 and its ligand Galectin9 are up-regulated in poor survivors compared to poor survivors (FIG. 26, panel (C)). TCGA model can “predict” the bad HCCs identified in the R01 data are indeed bad (red squares in panel (D)).

The “Bad” HCCs were Fatty-Warburg.

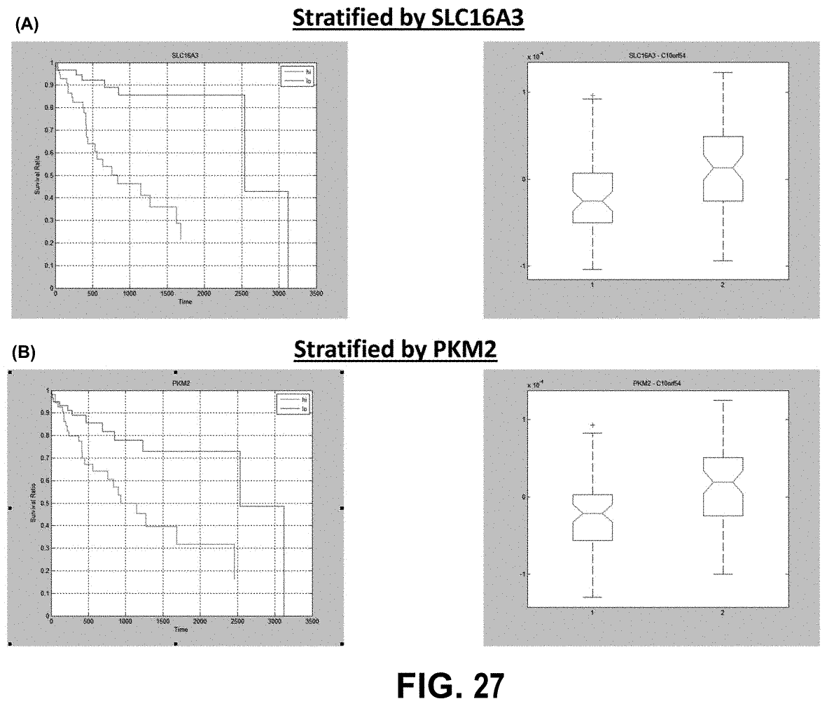

Bad HCCs were Warburgian. SLC2A1 (Glut1) supergene is up-regulated on the bad HCCs (glucose uptake). FIG. 27, panel (A) shows that SLC16A3 supergene was up-regulated on the bad HCCs (transports lactate, a by-product of aerobic glycolysis, out of hepatocyte). FIG. 27, panel (B) shows PKM2 supergene was up-regulated on bad HCCs (activates aerobic glycolysis).

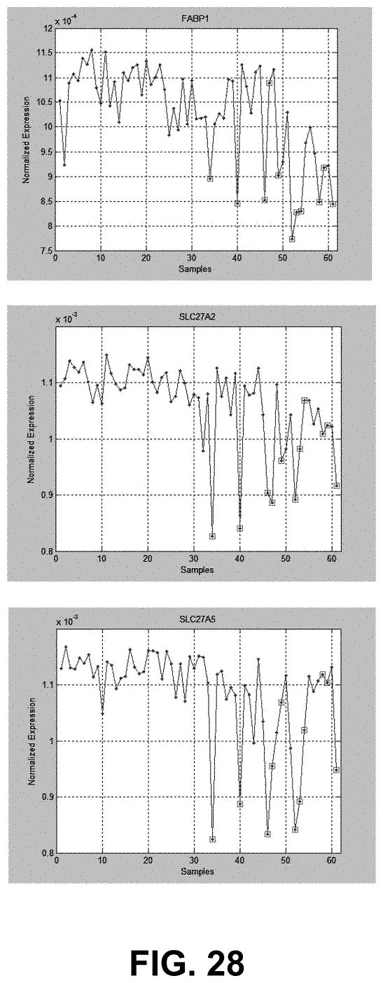

Bad HCCs synthesize fatty acids (FAs). Warburg effect diverts glycolytic metabolites to lipid synthesis. FIG. 28 shows that fatty acid uptake genes, FABP1 (Fatty Acid Binding Protein 1), SLC17A2 (FATP2, Fatty acid transporter 2), and SLC27A5 (Fatty acid transporter 5) were down-regulated in Fatty-Warburg HCCs. SLC17A2 (Fatty acid transporter 2) is a member of the FATP family of fatty acid uptake mediators, has independently been identified as a hepatic peroxisomal very long-chain acyl-CoA synthetase (VLACS). SLC27A5 (Fatty acid transporter 5) supports the efficient hepatocellular uptake of LCFAs, and thus liver lipid homeostasis is largely a protein-mediated process requiring FATP5. The data suggest that Fatty-Warburg tumors may be synthesizing FAs internally via glycolytic pathways. Treatments can target Warburgian, fatty acid or immune checkpoint pathways.

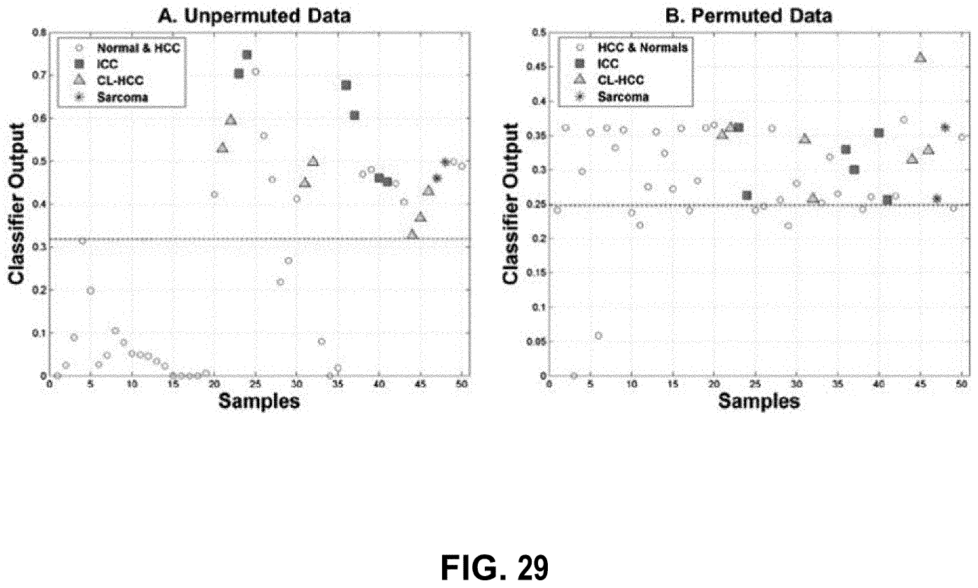

FIG. 29, panel (A) visualizes the output of a GRNN trained on the K₁/k₂ parameter for the 50 samples included in this study. Samples included in the expression phenotype Γ⁽⁻⁾ are highlighted by red squares (ICC), green triangles (CCL-HCC) and black asterisks (sarcoma) while the samples in Γ⁽⁺⁾ (adjacent-normal and HCC) are highlighted as blue circles. The horizontal threshold (magenta line) was used to classify each of the 50 samples by assigning a sample to the Γ⁽⁻⁾ expression phenotype if its GRNN value was greater than the threshold and to Γ⁽⁺⁾ otherwise. The GRNN trained on the denoised K₁/k₂ vector correctly classified all the samples in Γ⁽⁻⁾ and 71% of the samples in Γ⁽⁺⁾ for an average correct classification rate of 86%, which is significantly greater than chance. The GRNN output vector was significantly correlated with the target values in T (r=0.61267 p=1.987E-06). To assess the robustness of this result, the K₁/k₂ parameter vector was randomly permuted 1000 times and a GRNN was trained on each permutation using the target vector T and spread parameter equal to 0.23235. FIG. 29, panel (B) shows that it is difficult to separate Γ⁽⁻⁾ and Γ⁽⁺⁾ using any threshold in the output of a GRNN trained on a random permutation of the K₁/k₂ parameter vector, which is reflected in the low correlation of the GRNN output with the target vector T (r=0.27615, p=0.05223). Out of 1000 permutations only one had correlation greater than r=0.61, which resulted in a highly significant empirical p-value of p_{K}_{₁}_{/k}_{₂}. Hence, the observed separation of samples in Γ⁽⁻⁾ and Γ⁽⁺⁾ shown in FIG. 29, panel (A) was unlikely the result of a chance event.

These results suggests that the non-invasive monitoring of specific biological processes over time in liver tumors using PET imaging is possible. Note the K₁/k₂ parameter is just one quantitative feature that can be extracted from imaging data for the supervised analysis of multiple, big genomic data sets. The biological insight provided by correlative molecular signatures could be used to define imaging phenotypes of clinical significance that serve as targets in data-driven models that classify new samples based on image features. Relating predictive signatures extracted from molecular images to global patterns of genomic, transcriptomic, epigenomic and metabolomic variation using bioinformatic approaches such as JAMMIT can be referred to as “imaging genomics”. The central hypothesis of imaging genomics is that image features that capture variation over space and time reflect underlying genetic programs of biological and clinical relevance.

Altogether, these data revealed a novel sub-type of HCC with an expression signature similar to that of ICC, a liver cancer sub-type with a much poorer clinical outcome. This expression signature was found to be related to the wide-spread down-regulation of membrane lipid transport activity by IPA. This result is significant since difference in clinical outcome between these two tumor subtypes may be due in part to membrane transport inactivation. Notably, this application of JAMMIT showed the analysis of a big data matrix can be supervised by an arbitrary univariate function using l₁ regularization.

### Example 4

### Sparse Signal Processing of Multi-Modal Data and Precision Immunotherapy

This example describes predicting response to TIM-3 blockade in hepatocellular carcinoma (HCC) and intra-hepatic cholangiocarcinoma (CCA). This example also demonstrates sparse signal processing of multi-Modal data and precision immunotherapy to determine gene signatures for liver cancer and ovarian cancer.

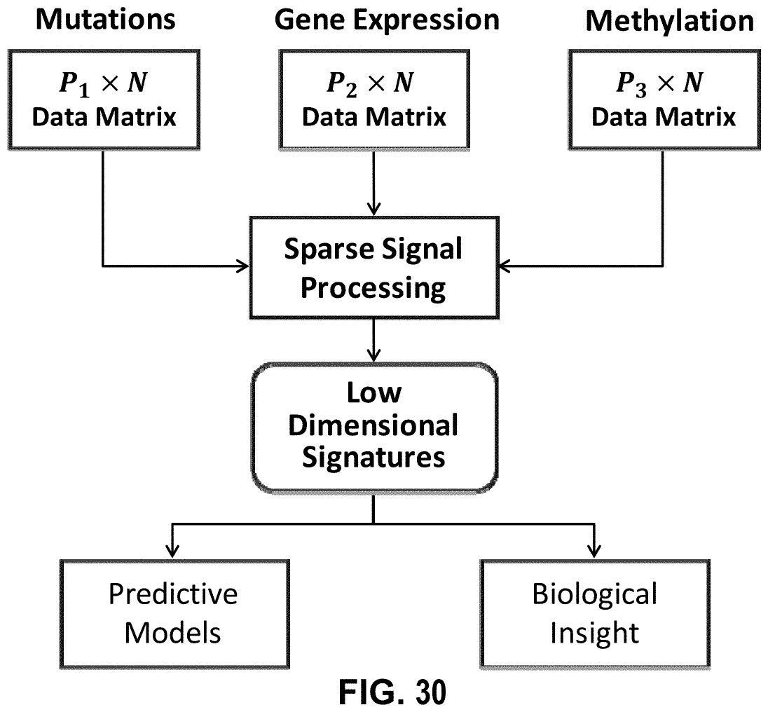

The analytical approach for predicting response to TIM-3 blockade can include the JAMMIT analysis (FIG. 30), which processes mutation data, gene expression data, and methylation data into low dimensional signatures using sparse signal processing. The low dimensional signatures can be used to generate predictive models and biological insight.

Immune Checkpoint Genes.

Interaction between immune checkpoint genes and their ligands (e.g., Programmed cell death-1 (PD-1)/Programmed death-ligand 1 (PD-L1)) can enable tumors to evade attack by killer T cells. Tumors may evade patient's immune system by activating immune checkpoint genes. Blocking these interactions has resulted in extraordinary survival profiles for many cancers. Unfortunately, costly immune checkpoint blockade (ICB) works in a small fraction of patients (e.g., only 10-40% of patients depending on the cancer). Thus, there is a need to objectively identify patients who will derive a survival benefit from ICB. One approach relies on the analysis of big biodata using algorithms to generate targeted signatures which correlate to ICB success.

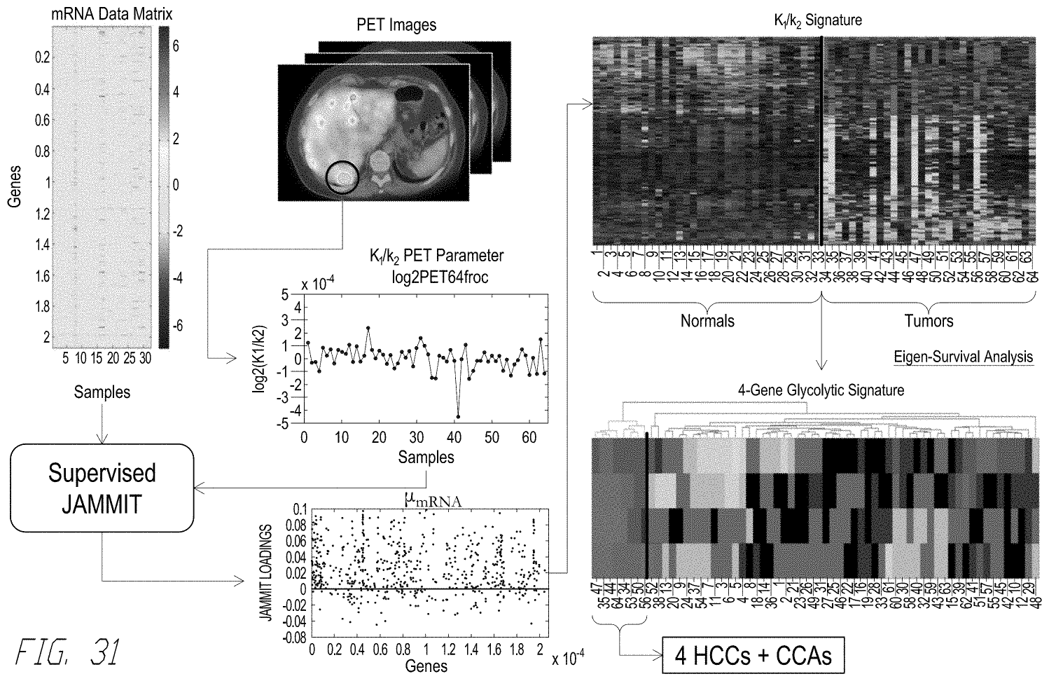

JAMMIT analysis of liver cancer. From a dataset that included 33 normal samples, 27 HCC samples, and 4 CCA samples, liver cancer signatures that were multi-modal and predictive of clinical outcomes (e.g., response to treatment) were determined using JAMMIT. The liver signatures discovered were validated using a The Cancer Genome Atlas (TCGA) dataset that included 260 HCC samples. Global mRNA, miRNA and DNA methylation data from TCGA included 390 tumors from patients with stage 3 disease; 291 tumors for discovery; 99 for validation; and censored survival data, clinical stage, etc. All patients received adjuvant platinum-based chemotherapy with taxane. FIG. 31 shows an example workflow used to determine multi-modal cancer signatures that were predictive of clinical outcome.

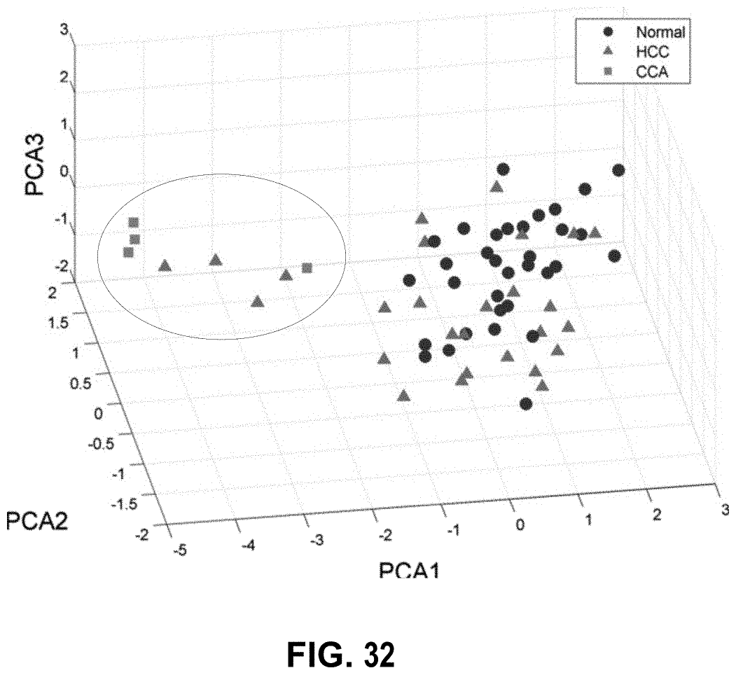

FIG. 32 is a non-limiting exemplary plot showing that an aggressive tumor subtype was identified by the 4-gene glycolytic signature. The liver cancer signature included the following genes: SLC16A3, PKM2, SPP1, and HK2. The 4 circled HCCs were co-clustered with the 4 circled CCAs. All 8 tumors were “Warburgian” in nature (e.g., high aerobic glycolysis). Like CCA, a Warburgian HCC is more aggressive than “standard” HCC. The 4-gene signature establishes a connection between the Warburg effect and immune checkpoint blockade

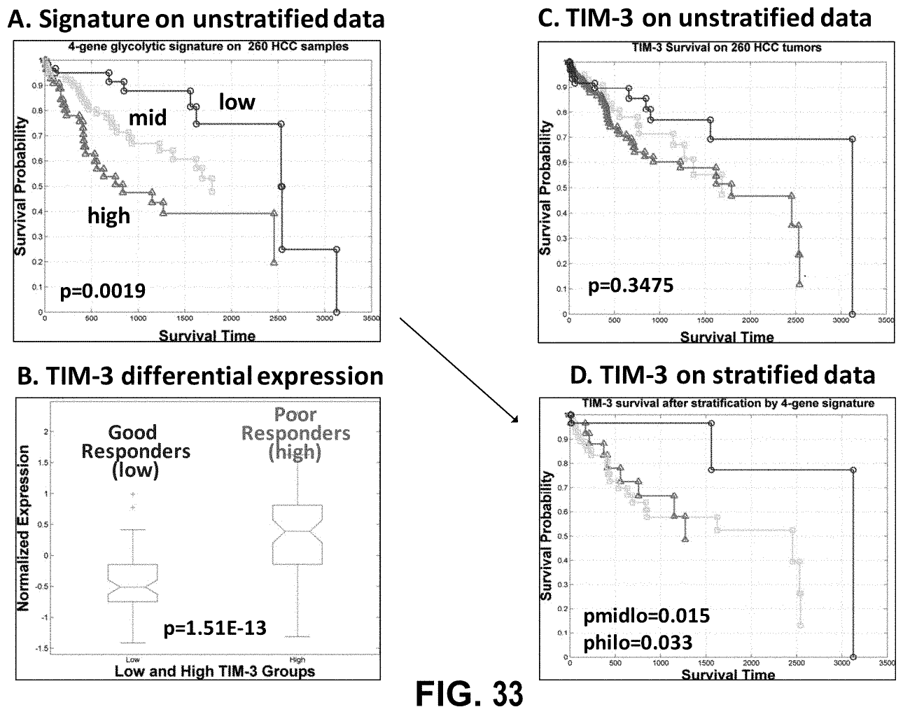

FIG. 33, panels (A)-(D) are plots showing that the 4-gene signature was used to identify a subset of patients who may derive a survival benefit from TIM-3 blockade. TIM3 expression was much higher on the poor prognosis group (red) than the good prognosis group. The 4-gene “glycolytic” signature was prognostic in a cohort of 260 HCC patients. TIM-3 was not prognostic on the same data set. The 4-gene signature identified a subset of patients for which TIM-3 was prognostic. Thus, the 4-gene signature was predictive of response to TIM-3 blockade. Since HCC and CCA co-cluster, CCA could also respond to TIM-3 blockade.

Applications.

Applications of the 4-gene signature includes prospective validation of 4-gene signature in clinical trial that combines anti-PD-L1 and anti-TIM-3 therapy. Further retrospective validation of glycolytic signature on independent data from TCGA and other data sources. The method can be used to identify signatures for other cancers in TCGA that predict response to TIM-3 blockade.

JAMMIT Analysis of Ovarian Cancer.

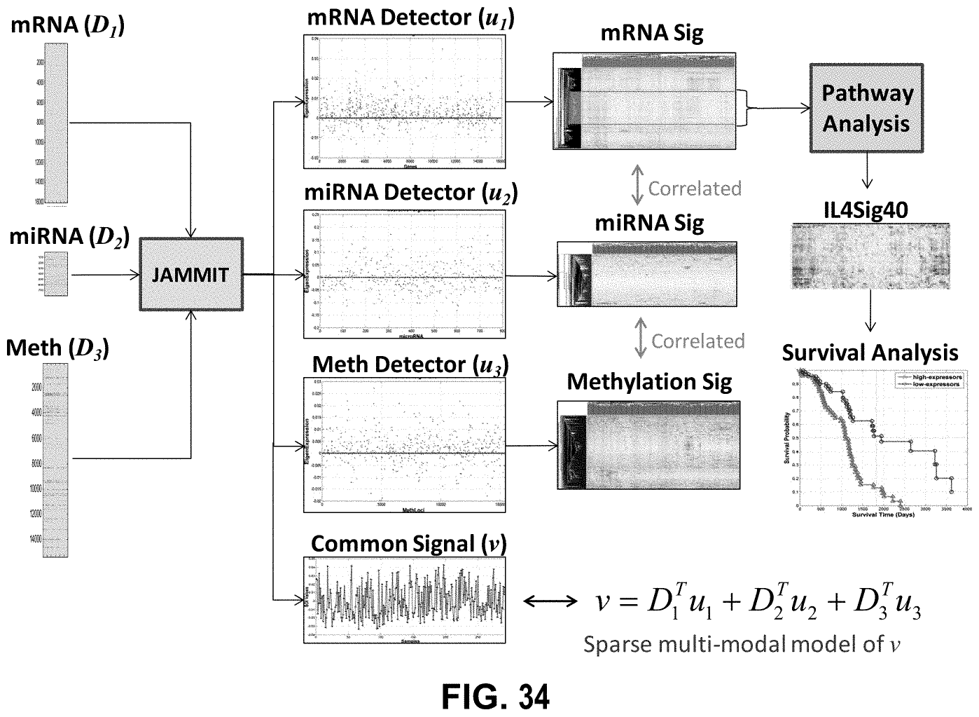

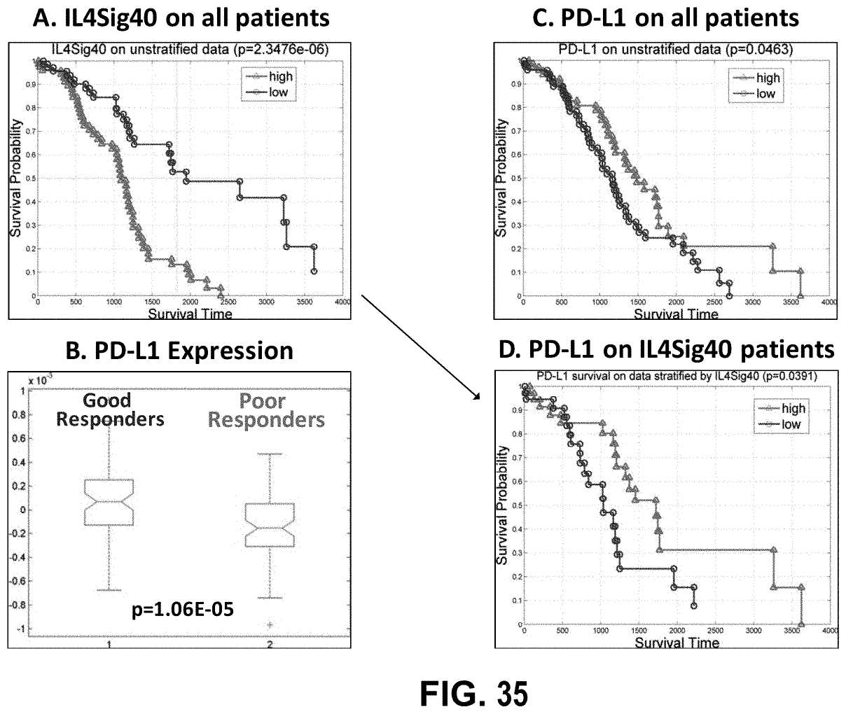

FIG. 34 shows an example JAMMIT workflow for analysis of ovarian cancer. The JAMMIT workflow illustrated in FIG. 34 was used to show that IL4Sig40 identifies a subset of patients who may derive a survival benefit from PD-L1 blockade (FIG. 35). The ovarian cancer signature includes one or more of the following genes: ACSL1, ALOX5AP, BCL3, CCL2, CCL5, CCR7, CD2, CD33, CD38, CD69, CD86, CST7, CXCR3, FASLG, FCGR2A, GBP2, HCLS1, ICOS, IFI30, IFNG, IL15, IL15RA, IL18RAP, IL1B, IL2RB, IL4R, ITGB2, KLRB1, LGALS1, MAF, PDE4B, SELE, SELPLG, SLAMF7, TBX21, TFEC, TGFBI, TLR4, TNFRSF9, and VDR

The IL4Sig40 signature was an independent predictor for overall survival in the study cohort of 291 ovarian cancer patients from TCGA. Expression patterns of cytokines and checkpoint genes suggested that good responders have highly immunogenic tumor microenvironments (TMEs) that were held in check by the up-regulation of multiple checkpoint genes (PD-1, PD-L1, CTLA4, etc.) when compared to the poor responders. The up-regulation of PD-L1 expression in the good responders may be associated in some way with better overall survival. PD-L1 on the unstratifed cohort may be only nominally associated with survival independent of IL4Sig40. PD-L1 on the cohort stratified by ILSig40 was significantly associated with survival. So “filtering” of the study cohort by ILSig40 results in a subset of patients that could derive a survival benefit by blockade of PD-L1. Thus, IL4Sig40 characterized highly immunogenic microenvironments that are held in check by the up-regulation of multiple checkpoint genes. IL4Sig40 identified a subset of ovarian cancer patients who should derive a survival benefit by combining platinum-based chemotherapy and anti-PD-1/PD-L1 therapy.

Altogether, these data demonstrate that sparse signal processing (SSP) can result in multi-modal signatures that are predictive of response to immune checkpoint blockade for ovarian and liver cancer. And sparse matrix approximations of rank-1 can be a simple and effective approach to implementing SSP for precision medicine applications.

### Terminology

In at least some of the previously described embodiments, one or more elements used in an embodiment can interchangeably be used in another embodiment unless such a replacement is not technically feasible. It will be appreciated by those skilled in the art that various other omissions, additions and modifications may be made to the methods and structures described herein without departing from the scope of the claimed subject matter. All such modifications and changes are intended to fall within the scope of the subject matter, as defined by the appended claims.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity. As used in this specification and the appended claims, the singular forms “a,” “an,” and “the” include plural references unless the context clearly dictates otherwise. Any reference to “or” herein is intended to encompass “and/or” unless otherwise stated.

It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and/or “an” should be interpreted to mean “at least one” or “one or more”); the same holds true for the use of definite articles used to introduce claim recitations. In addition, even if a specific number of an introduced claim recitation is explicitly recited, those skilled in the art will recognize that such recitation should be interpreted to mean at least the recited number (e.g., the bare recitation of “two recitations,” without other modifiers, means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, such as in terms of providing a written description, all ranges disclosed herein also encompass any and all possible sub-ranges and combinations of sub-ranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like include the number recited and refer to ranges which can be subsequently broken down into sub-ranges as discussed herein. Finally, as will be understood by one skilled in the art, a range includes each individual member. Thus, for example, a group having 1-3 articles refers to groups having 1, 2, or 3 articles. Similarly, a group having 1-5 articles refers to groups having 1, 2, 3, 4, or 5 articles, and so forth.

While various aspects and embodiments have been disclosed herein, other aspects and embodiments will be apparent to those skilled in the art. The various aspects and embodiments disclosed herein are for purposes of illustration and are not intended to be limiting, with the true scope and spirit being indicated by the following claims.

1.-75. (canceled)

76. A method for developing a targeted immunogenic gene signature for immune checkpoint blockade (ICB) using a machine learning model, the method comprising:
receiving data on a plurality of expression patterns associated with a plurality of realizations of a targeted signature determined using a plurality of tissue samples of a cancer, wherein the targeted signature is indicative of a plurality of genes that depends on a checkpoint gene and the cancer, and wherein the realization of the targeted signature is associated with an observed outcome determination of a plurality of outcome determinations;
generating a training dataset comprising a plurality of exemplars by factoring the plurality of realizations;
training a machine learning model using the training dataset; and
determining a predicted outcome determination of a plurality of outcome determinations of a second tumor type using the machine learning model and a realization of the immunogenic gene signature.

77. The method according to claim 76, wherein the immune checkpoint blockade comprises at least one of a CTLA-4 blockade or a PD-1 blockade.

78. The method according to claim 76, wherein the cancer comprises at least one of an ovarian cancer or a liver cancer.

79. The method according to claim 76, wherein the plurality of outcome determinations comprises a responsive group, a non-responsive group, and an uncertain outcome group.

80. The method according to claim 79, wherein the checkpoint gene is differentially expressed between the responsive group and the non-responsive group.

81. The method according to claim 76, wherein the plurality of realizations is factored using single value decomposition (SVD) to generate an eigen-survival model (ESM).

82. The method according to claim 81, where a realization of the immunogenic signature of the second tumor type is projected into the eigen-survival model to generate a prognostic score for the second tumor type.

83. The method according to claim 82, wherein the realization of the immunogenic gene signature of the second tumor type is prognostic in a subset of the plurality of patients of the plurality of tissue samples restricted to the responsive group and the non-responsive group.

84. The method according to claim 76, wherein the secondary tumor type comprises an malignant melanoma.
